SEQUENCE LISTING (74 sequences)

SEQ ID NO: 1 - protein, 536 aa, Homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGLSSGEPPEKERRRLKESFENYRRKRALRKMQKGWRQGEEDRENTTGSDNTDTEGS

SEQ ID NO: 2 - protein, 536 aa, Mus musculus
MSDYENDDECWSTLESFRVKLISVIDPSRITPYLRQCKVLNPDDEEQVLSDPNLVIRKRKVGVLLDILQRTGHKGYVAFLESLELYYPQLYRKVTGKEPARVFSMIIDASGESGLTQLLMTEVMKLQKKVQDLTALLSSKDDFIKELRVKDSLLRKHQERVQRLKEECELSSAELKRCKDENYELAMCLAHLSEEKGAALMRNRDLQLEVDRLRHSLMKAEDDCKVERKHTLKLRHAMEQRPSQELLWELQQEKDLLQARVQELQVSVQEGKLDRNSPYIQVLEEDWRQALQEHQKQVSTIFSLRKDLRQAETLRARCTEEKEMFELQCLALRKDAKMYKDRIEAILLQMEEVSIERDQAMASREELHAQCTQSFQDKDKLRKLVRELGEKADELQLQLFQTESRLLAAEGRLKQQQLDMLILSSDLEDSSPRNSQELSLPQDLEEDAQLSDKGVLADRESPEQPFMALNKEHLSLTHGMGPSSSEPPEKERRRLKESFENYRRKRALRKMQNSWRQGEGDRGNTTGSDNTDTEGS

SEQ ID NO: 3 - DNA, 2111 bp, Homo sapiens
  source 1..2111: mol_type = genomic DNA
aagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggctgccttgccggcggggggagcccgaaacagccctttgcagctctgcaccaggagcaggttttgcggaacccccatgacgcaggcctgagcagcggggagccgcccgagaaggagcggcggcgcctcaaagagagttttgagaactaccgcaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca

SEQ ID NO: 4 - DNA, 1871 bp, Mus musculus
  source 1..1871: mol_type = genomic DNA
ctccttcatggctccacccttctccagttagggaacccctccacactcccagagacccaggctcctggtatgtccataacccagacagcatctgctggcaggtagctctcacaagaccctgagcctacagaggacatgtcagactatgagaatgacgacgagtgctggagcaccctggagagcttccgggtgaagctcatctctgtcattgacccctcccggatcacaccctatctacgccagtgcaaagtcctgaaccccgatgatgaggagcaggtgctcagtgaccccaacctggtcatccgcaagcggaaagtgggtgtgctcctggacatcctgcagcggacaggccacaagggctacgtggctttcctcgagagcctggagctctactaccctcagttataccggaaagtcactggcaaggagccagcacgcgtcttctccatgatcattgatgcatcaggggagtctggcctgacgcagctgctgatgacagaggtcatgaagctgcagaagaaggttcaggacctgacggcccttctgagctccaaggacgacttcatcaaggagctgagggtaaaggacagcctactgcgcaagcaccaggagcgggtgcagcggctcaaggaggagtgtgagctgagcagtgcggagctgaagcgctgcaaggacgagaactatgagctggccatgtgcctggcacatctgagtgaagagaagggcgcagcactcatgcggaaccgtgacctgcagcttgaggtggaccggctcaggcacagcctcatgaaggccgaggatgactgcaaggtggagcgcaaacacacactgaagctcaggcacgccatggagcagcggcctagtcaggagctgctgtgggaactacagcaggaaaaggacttgctgcaggcccgggtgcaggagctgcaggtctctgtgcaggagggtaagctagacaggaatagtccatacattcaagtgctggaggaggactggcgtcaagcactgcaggaacaccagaagcaggtcagcaccatcttctccctacggaaggacctccgccaggctgagaccctccgggcccggtgcacggaagaaaaggagatgttcgagctgcagtgcctggccttgcgcaaggatgccaagatgtacaaggaccgtatcgaggctatcctgctgcagatggaggaggtctccattgagagggaccaggctatggcctccagggaagagctgcatgcacagtgtacccaaagctttcaggacaaagataagcttcgaaagctggttcgagagctgggtgagaaggcagatgagctgcagctacagctgttccagacggagagccgattactggccgccgagggcagactcaagcagcagcaattggacatgctcatcctgagctctgacttggaagacagttcacccaggaactcccaggagctctcactgcctcaggatctggaggaggatgcccagctctcagacaaaggtgtactggcagacagggagagcccagagcagccctttatggctctgaacaaggagcatctttcactgacccatggcatggggcccagcagcagcgagcccccggagaaggagcggcggcgcctcaaggagagcttcgagaactaccgcaggaagcgggcgctccgcaagatgcagaacagctggcggcagggagaaggggatcgcgggaatacgacaggcagcgacaacaccgacaccgagggctcctagcgaaccgcgccgaggctgagcatctgtggaattgtgaaaggatgctgcggtttttttttttttttttttttttttactgtattagaattagaaaatgcaactaaataaaataatcaccgagctga

SEQ ID NO: 5 - protein, 165 aa, synthetic construct
MGWSWIFLFLLSGTAGVHSEVQLQQSGPELVKPGASMKMSCKASGYSFTGQTMHWVKQTHGKNLEWIGLINPYNGGTKYNQIFTGKATLTVDKSSSSAYMELLSLTSEDSAVYFCAIWDYVLDYWGQGTSVTVSSAKTTPPSVYPLAPGSAAQTNSMVTLGCLVK

SEQ ID NO: 6 - protein, 8 aa, synthetic construct
GYSFTGQT

SEQ ID NO: 7 - protein, 8 aa, synthetic construct
INPYNGGT

SEQ ID NO: 8 - protein, 11 aa, synthetic construct
CAIWDYVLDYW

SEQ ID NO: 9 - protein, 159 aa, synthetic construct
MKLPVRLLVLMFWIPASSSDVVMTQTPLSLPVSLGDQASISCRSSQSLLHSNGKTYLHWYLQKPGQSPKLLIYKVSNRFSGVPDRFSGSGSGTDFTLKISRVEAEDLGVYFCSQSTHIPPTFGGGTKLEIKRADAAPTVSIFPPSSEQLTSGGASVVCF

SEQ ID NO: 10 - protein, 11 aa, synthetic construct
QSLLHSNGKTY

SEQ ID NO: 11 - protein, 11 aa, synthetic construct
CSQSTHIPPTF

SEQ ID NO: 12 - DNA, 20 bp, synthetic construct
  source 1..20: mol_type = other DNA
tacttcctcctcaagaacga

SEQ ID NO: 13 - DNA, 20 bp, synthetic construct
  source 1..20: mol_type = other DNA
ctcaggcgtttccacatgga

SEQ ID NO: 14 - DNA, 20 bp, synthetic construct
  source 1..20: mol_type = other DNA
ggtggtgcatcgtgacctga

SEQ ID NO: 15 - DNA, 20 bp, synthetic construct
  source 1..20: mol_type = other DNA
gatatacaggatgacgccta

SEQ ID NO: 16 - DNA, 20 bp, synthetic construct
  source 1..20: mol_type = other DNA
acttcacgacagtatatcgg

SEQ ID NO: 17 - DNA, 20 bp, synthetic construct
  source 1..20: mol_type = other DNA
attacatacatcgtaagctc

SEQ ID NO: 18 - DNA, 20 bp, synthetic construct
  source 1..20: mol_type = other DNA
atgatcgtataccctaccag

SEQ ID NO: 19 - DNA, 20 bp, synthetic construct
  source 1..20: mol_type = other DNA
aggcgatgcacatatgacca

SEQ ID NO: 20 - DNA, 20 bp, synthetic construct
  source 1..20: mol_type = other DNA
taatggagtacctcgctggt

SEQ ID NO: 21 - DNA, 20 bp, synthetic construct
  source 1..20: mol_type = other DNA
tatctctgcagatttacaca

SEQ ID NO: 22 - DNA, 20 bp, synthetic construct
  source 1..20: mol_type = other DNA
cattctagtgaactcacgtg

SEQ ID NO: 23 - DNA, 20 bp, synthetic construct
  source 1..20: mol_type = other DNA
tgcaactccccgtacatcgt

SEQ ID NO: 24 - DNA, 20 bp, synthetic construct
  source 1..20: mol_type = other DNA
catcctggtgaactctcgcg

SEQ ID NO: 25 - DNA, 20 bp, synthetic construct
  source 1..20: mol_type = other DNA
gtgcaactcgccctacatcg

SEQ ID NO: 26 - DNA, 20 bp, synthetic construct
  source 1..20: mol_type = other DNA
aaagtgcggcatgctcagag

SEQ ID NO: 27 - DNA, 20 bp, synthetic construct
  source 1..20: mol_type = other DNA
tccgggagtccaggttccgg

SEQ ID NO: 28 - DNA, 20 bp, synthetic construct
  source 1..20: mol_type = other DNA
tggatttggatgtagtaatg

SEQ ID NO: 29 - DNA, 20 bp, synthetic construct
  source 1..20: mol_type = other DNA
aaatggtccacaaaccaagt

SEQ ID NO: 30 - DNA, 20 bp, synthetic construct
  source 1..20: mol_type = other DNA
agaggctgcagagataatgc

SEQ ID NO: 31 - DNA, 20 bp, synthetic construct
  source 1..20: mol_type = other DNA
acttctggccagagcgccga

SEQ ID NO: 32 - DNA, 20 bp, synthetic construct
  source 1..20: mol_type = other DNA
ggtgtgctcaaaatcagatg

SEQ ID NO: 33 - DNA, 20 bp, synthetic construct
  source 1..20: mol_type = other DNA
taccaggaggagtgaaactc

SEQ ID NO: 34 - DNA, 20 bp, synthetic construct
  source 1..20: mol_type = other DNA
cttgctctagggctacaaca

SEQ ID NO: 35 - DNA, 20 bp, synthetic construct
  source 1..20: mol_type = other DNA
cagggagtgcagatactcaa

SEQ ID NO: 36 - DNA, 20 bp, synthetic construct
  source 1..20: mol_type = other DNA
gtggataagtccatagagca

SEQ ID NO: 37 - DNA, 20 bp, synthetic construct
  source 1..20: mol_type = other DNA
aaccacaaaacagcaaactg

SEQ ID NO: 38 - DNA, 20 bp, synthetic construct
  source 1..20: mol_type = other DNA
agtgttccctcaatccagag

SEQ ID NO: 39 - DNA, 20 bp, synthetic construct
  source 1..20: mol_type = other DNA
aggtaggactggagtacgtg

SEQ ID NO: 40 - DNA, 20 bp, synthetic construct
  source 1..20: mol_type = other DNA
agaaggtggcgataaactcg

SEQ ID NO: 41 - DNA, 20 bp, synthetic construct
  source 1..20: mol_type = other DNA
agcccaccatgtatcctgag

SEQ ID NO: 42 - DNA, 20 bp, synthetic construct
  source 1..20: mol_type = other DNA
tcccgactacatatcgcccg

SEQ ID NO: 43 - DNA, 20 bp, synthetic construct
  source 1..20: mol_type = other DNA
agcacggttaagaaaaaccc

SEQ ID NO: 44 - DNA, 20 bp, synthetic construct
  source 1..20: mol_type = other DNA
aagggggcttgatctcacgc

SEQ ID NO: 45 - DNA, 20 bp, synthetic construct
  source 1..20: mol_type = other DNA
aggaccaccagtctgcactg

SEQ ID NO: 46 - DNA, 20 bp, synthetic construct
  source 1..20: mol_type = other DNA
ccagaagtagttaaccgcag

SEQ ID NO: 47 - DNA, 20 bp, synthetic construct
  source 1..20: mol_type = other DNA
ccagttgcaggcttaaaagg

SEQ ID NO: 48 - DNA, 20 bp, synthetic construct
  source 1..20: mol_type = other DNA
gcgggagcatgaacgcaaag

SEQ ID NO: 49 - DNA, 20 bp, synthetic construct
  source 1..20: mol_type = other DNA
cctgaagtagtaaacagacg

SEQ ID NO: 50 - DNA, 20 bp, synthetic construct
  source 1..20: mol_type = other DNA
gggcgaggagctgttcaccg

SEQ ID NO: 51 - DNA, 20 bp, synthetic construct
  source 1..20: mol_type = other DNA
gagctggacggcgacgtaaa

SEQ ID NO: 52 - protein, 14 aa, synthetic construct
  SITE 1: note = Acetylated residue
  SITE 8: note = Phosphorylated residue
  SITE 14: note = Amidated residue
CEPARVFSMIIDAS

SEQ ID NO: 53 - protein, 14 aa, synthetic construct
  SITE 1: note = Acetylated residue
  SITE 14: note = Amidated residue
CEPARVFSMIIDAS

SEQ ID NO: 54 - protein, 13 aa, synthetic construct
  SITE 7: note = Phosphorylated residue
  SITE 13: note = Amidated residue
EPACVFSMIIDAS

SEQ ID NO: 55 - protein, 151 aa, synthetic construct
SDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDS

SEQ ID NO: 56 - protein, 151 aa, synthetic construct
SDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIEDASGESGLTQLLMTEVMKLQKKVQDLTALLSSKDDFIKELRVKDS

SEQ ID NO: 57 - protein, 151 aa, synthetic construct
SDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFDMIIDASGESGLTQLLMTEVMKLQKKVQDLTALLSSKDDFIKELRVKDS

SEQ ID NO: 58 - protein, 151 aa, synthetic construct
SDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPACVFSMIIDASGESGLTQLLMTEVMKLQKKVQDLTALLSSKDDFIKELRVKDS

SEQ ID NO: 59 - protein, 151 aa, synthetic construct
SDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPACVFDMIIDASGESGLTQLLMTEVMKLQKKVQDLTALLSSKDDFIKELRVKDS

SEQ ID NO: 60 - protein, 231 aa, synthetic construct
MEPTAPSLTEEDLTEVKKDALENLRVYLAEKIIAERHFDHLRAKKILSREDTEEISARTSSRKRAGKLLDYLQENPKGLDTLVESIRREKTQNFLIQKITDEVLKLRNIKLEHLKGLKCSSCEPFPDGATNNLSRSNSDESNFSEKLRASTVMYHPEGESSTTPFFSTNSSLNLPVLEVGRTENTIFSSTTLPRPGDPGAPPLPPDLQLEEEGTCANSSEMFLPLRSRTVS

SEQ ID NO: 61 - protein, 8 aa, Homo sapiens
PARVFSMI

SEQ ID NO: 62 - protein, 8 aa, Mus musculus
PARVFSMI

SEQ ID NO: 63 - protein, 8 aa, Danio rerio
PARVFSVL

SEQ ID NO: 64 - protein, 8 aa, Xenopus tropicalis
PTRVFSMI

SEQ ID NO: 65 - protein, 8 aa, Homo sapiens
PTRRFSTI

SEQ ID NO: 66 - protein, 8 aa, Mus musculus
PTRRFSTI

SEQ ID NO: 67 - protein, 8 aa, Danio rerio
PTRRCSTI

SEQ ID NO: 68 - protein, 8 aa, Xenopus tropicalis
PTRRFSTI

SEQ ID NO: 69 - DNA, 300 bp, Mus musculus
  source 1..300: mol_type = genomic DNA
cctccacccgccagcccttggccttgactctgtcttccacctgaaggtgtgctcctggacatcctgcagcggacaggccacaagggctacgtggctttcctcgagagcctggagctctactaccctcagttataccggaaagtcactggcaaggagccagcacgcgtcttctccatgatcattggtgagaggcacgggtgtgggttgttgctgtgggtggactcgggtcctggcttctatgatggcgttggagtgactaagtgtgataacaagccttacgatgtagatcagggtagtggt

SEQ ID NO: 70 - DNA, 181 bp, Mus musculus
  source 1..181: mol_type = genomic DNA
acaggccacaagggctacgtggctttcctcgagagcctggagctctactaccctcagttataccggaaagtcactggcaaggagccagcatgcgtcttctcaatgatcattggtgagaggcacgggtgtgggttgttgctgtgggtggactcgggtcctggcttctatgatggcgttggag

SEQ ID NO: 71 - DNA, 23 bp, synthetic construct
  source 1..23: mol_type = other DNA
ccatgatcattggtgagaggcac

SEQ ID NO: 72 - DNA, 21 bp, unidentified
  source 1..21: mol_type = unassigned DNA
cagcacgcgtcttctccatga

SEQ ID NO: 73 - DNA, 21 bp, unidentified
  source 1..21: mol_type = unassigned DNA
cagcatgcgtcttctccatga

SEQ ID NO: 74 - DNA, 22 bp, unidentified
  source 1..22: mol_type = unassigned DNA
cagcatgcgtcttctcaatgat

(19) United States
(12) Patent Application Publication
(10) Pub. No.: US 2026/0158139 A1
(43) Pub. Date: Jun. 11, 2026

(54) CARD9 VARIANT POLYPEPTIDE AND ANTIBODIES DIRECTED THERETO

(71) Applicants: The Broad Institute, Inc., Cambridge, MA (US); The General Hospital Corporation, Boston, MA (US)

(72) Inventors: Ramnik Xavier, Cambridge, MA (US); Daniel Graham, Cambridge, MA (US); Zhifang Cao, Boston, MA (US); Marta Brandt, Cambridge, MA (US)

(73) Assignees: The Broad Institute, Inc., Cambridge, MA (US); The General Hospital Corporation, Boston, MA (US)

(21) Appl. No.: 19/537,265

(22) Filed: Feb. 11, 2026

Related U.S. Application Data
(63) Continuation of application No. PCT/US2024/043550, filed on Aug. 23, 2024.
(60) Provisional application No. 63/534,722, filed on Aug. 25, 2023.

(51) Int. Cl. A61K 39/395; A61K 45/06; C07K 16/18

(52) U.S. Cl. CPC A61K 39/3955; A61K 45/06; C07K 16/18

(57) ABSTRACT

Described and featured herein are antibodies that specifically bind to a phosphorylated S104 amino acid residue in a CARD9 polypeptide and methods of using such antibodies to identify subjects vulnerable to fungal infection, including fungal infections caused by Candida spp.

### CROSS REFERENCE TO RELATED APPLICATIONS

This application is a continuation under 35 U.S.C. § 111(a) of PCT International Patent Application No. PCT/US2024/043550, filed Aug. 23, 2024, designating the United States and published in English, which claims priority to and the benefit of U.S. Provisional Application No. 63/534,722, filed Aug. 25, 2023, the entire contents of each of which are incorporated by reference herein.

### STATEMENT OF RIGHTS TO INVENTIONS MADE UNDER FEDERALLY SPONSORED RESEARCH

This invention was made with government support under grant Nos. R01A1137325 and P30DK043351, awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Aug. 30, 2024 is named 167741-049801PCT_SL.xml, and is 74,956 bytes in size.

### BACKGROUND

Invasive fungal infections are one of the major causes of infectious-disease related mortality, and the available treatments are limited. Most cases are driven by opportunistic fungi, which can lead to life-threatening infections, especially in immunocompromised patients. Such infections can cause fungal sepsis and damage to vital organs such as kidneys, brain, liver, lungs or heart. The appearance of more drug-resistant fungal strains is also problematic in hospital and residential care facilities and represents a serious burden to patients and health care professionals. Given the increase in the number of patients immunocompromised due to chronic illnesses, medication or invasive medical procedures, as well as the emergence of drug resistant fungal species, there is an urgent need to identify such patients and to select them for aggressive fungal infection therapies.

### SUMMARY

As described below, the present disclosure features antibodies and antigen binding fragments thereof that specifically bind a phosphorylated S104 amino acid residue in a CARD9 polypeptide, and methods of using such antibodies and antigen binding fragments thereof for characterizing subjects, including identifying subjects having a propensity to develop a severe fungal infection, and selecting an appropriate therapy for such patients.

In an aspect, the present disclosure provides an antibody that specifically binds to a phosphorylated S104 amino acid residue in a caspase activation and recruitment domain 9 (CARD9) polypeptide or peptide, or an antigen binding portion thereof. The antibody includes a heavy chain variable domain having at least about 85% identity to CDR1, CDR2, and CDR3 and a light chain variable domain having at least about 85% identity to CDR1, CDR2, and CDR3 of Table 1.

In an aspect, the present disclosure provides an isolated nucleic acid molecule encoding the antibody or antigen binding fragment of any of the aspects of the present disclosure or embodiments thereof.

In an aspect, the present disclosure provides a vector including a nucleic acid sequence encoding the antibody of any of the aspects of the present disclosure or embodiments thereof.

In an aspect, the present disclosure provides a host cell including the vector of any of the aspects of the present disclosure or embodiments thereof.

In an aspect, the present disclosure provides a method of characterizing the activation state of a CARD9 polypeptide. The method involves contacting a biological sample with an antibody or an antigen-binding portion thereof, of any of the aspects of the present disclosure or embodiments thereof, and detecting or failing to detect binding of the antibody or an antigen-binding portion thereof to a phosphorylated or unphosphorylated S104 of CARD9 in the sample, thereby characterizing the activation state of a CARD9 polypeptide.

In an aspect, the present disclosure provides a method of treating a selected subject having a fungal infection. The method involves administering to the selected subject an aggressive anti-fungal therapy, where the subject is selected for treatment by detecting in a biological sample of the subject a reduction in the level of CARD9 polypeptide in which amino acid residue S104 is phosphorylated, relative to a reference.

In an aspect, the present disclosure provides method of selecting a subject having a propensity to develop a severe fungal infection. The method involves contacting a biological sample obtained from the subject with an antibody of any of the aspects of the present disclosure or embodiments thereof, or an antigen-binding portion thereof, that specifically binds to a phosphorylated S104 amino acid residue in a CARD9 polypeptide, detecting a reduction in binding levels of the antibody, or an antigen-binding portion thereof, to a phosphorylated S104 amino acid residue in the CARD9 polypeptide, relative to a reference, and selecting the subject as having a propensity to develop an impaired immune response associated with the fungal infection based on the detecting step.

In an aspect, the present disclosure provides a method of selecting a subject having, or having a propensity to develop, Crohn's disease, inflammatory bowel disease, ankylosing spondylitis, primary sclerosing cholangitis, or IgA nephropathy. The method involves contacting a biological sample obtained from the subject with an antibody of any of the aspects of the present disclosure or embodiments thereof, or an antigen-binding portion thereof, that specifically binds to a phosphorylated S104 amino acid residue in a CARD9 polypeptide, detecting an increase in binding levels of the antibody, or an antigen-binding portion thereof, to a phosphorylated S104 amino acid residue in the CARD9 polypeptide, relative to a reference, and selecting the subject as having, or having a propensity to develop, Crohn's disease, inflammatory bowel disease, ankylosing spondylitis, primary sclerosing cholangitis, or IgA nephropathy based on the detecting step.

In an aspect, the present disclosure provides a method of screening for an agent that activates a CARD9 polypeptide. The method involves contacting a sample with an agent and with the antibody or an antigen-binding portion thereof, of one or more of any of the aspects of the present disclosure or embodiments thereof, under conditions and for a time sufficient for binding of the antibody or an antigen binding portion thereof to bind to the CARD9 protein if present in the sample, and identifying the agent as an activator of the CARD9 polypeptide by detecting the binding of the antibody or an antigen binding portion thereof, to the CARD9 polypeptide in the sample compared with a control sample in which the CARD9 protein is absent or in which the CARD9 protein is unphosphorylated.

In an aspect, the present disclosure provides a method of screening for an agent that inhibits the activation of a CARD9 polypeptide. The method involves contacting a sample with an agent and with the antibody or an antigen-binding portion thereof, of one or more of any of the aspects of the present disclosure or embodiments thereof, under conditions and for a time sufficient for binding of the antibody or an antigen binding portion thereof to bind to the CARD9 protein if present in the sample, and identifying the agent as an inhibitor of the activation of the CARD9 polypeptide by detecting a reduction in the binding of the antibody or an antigen binding portion thereof, to the CARD9 polypeptide in the sample compared with a control sample in which the CARD9 protein is activated or in which the CARD9 protein is phosphorylated.

In any of the above aspects, or embodiments thereof, the antibody comprises a heavy chain variable domain having at least about 90% identity to CDR1, CDR2, and CDR3 and a light chain variable domain having at least about 90% identity to CDR1, CDR2, and CDR3 of Table 1.

In any of the above aspects, or embodiments thereof,the antibody comprises a heavy chain variable domain having at least about 95% identity to CDR1, CDR2, and CDR3 and a light chain variable domain having at least about 90% identity to CDR1, CDR2, and CDR3 of Table 1.

In any of the above aspects, or embodiments thereof, the antibody comprises a heavy chain variable domain comprising CDR1, CDR2, and CDR3 and a light chain variable domain comprising CDR1, CDR2, and CDR3 of Table 1.

In any of the above aspects, or embodiments thereof, the antibody includes an affinity tag or a detectable moiety.

In any of the above aspects, or embodiments thereof, the vector is an expression vector.

In any of the above aspects, or embodiments thereof, the expression vector is a viral or non-viral expression vector.

In any of the above aspects, or embodiments thereof, the vector further includes a nucleic acid sequence encoding an affinity tag or a detectable amino acid sequence operably linked to the polypeptide or antibody.

In any of the above aspects, or embodiments thereof, the antibody specifically binds a phosphorylated S104 amino acid residue present in CARD9, where the binding detects activated CARD9.

In any of the above aspects, or embodiments thereof, the antibody fails to bind a phosphorylated S104 amino acid residue present in CARD9, where the failure to bind detects unactivated CARD9.

In any of the above aspects, or embodiments thereof, the sample is obtained from a subject.

In any of the above aspects, or embodiments thereof, the biological sample includes bone marrow cells.

In any of the above aspects, or embodiments thereof, the biological sample includes one or more of: macrophages; dendritic cells; neutrophils; and monocytes.

In any of the above aspects, or embodiments thereof, detecting an activated CARD9 polypeptide indicates that the subject is capable of mounting an adequate immune response to a fungal infection.

In any of the above aspects, or embodiments thereof, failing to detect an activated CARD9 polypeptide indicates that the subject has a propensity to develop a severe fungal infection.

In any of the above aspects, or embodiments thereof, the detecting includes contacting a biological sample of the subject with the antibody of any one of the aspects of the present disclosure, or embodiments thereof, and detecting or failing to detect binding of the antibody to a CARD9 polypeptide present in the biological sample.

In any of the above aspects, or embodiments thereof, the sample is a tissue sample, a blood, serum, or plasma sample.

In any of the above aspects, or embodiments thereof, the sample is obtained from a wound or site of fungal infection.

In any of the above aspects, or embodiments thereof, the sample comprises bone marrow cells, bone marrow dendritic cells, myeloid cells, or T lymphocytes.

In any of the above aspects, or embodiments thereof, the anti-fungal treatment or treatment regimen comprises a high or elevated dose of an anti-fungal drug or combination of drugs.

In any of the above aspects, or embodiments thereof, the method further involves administering to the selected subject an immunosuppressive or immunomodulatory agent.

In any of the above aspects, or embodiments thereof, the sample is a cell.

In any of the above aspects, or embodiments thereof, the sample includes one or more of macrophages, dendritic cells, monocytes, or neutrophils.

Compositions and methods defined in this disclosure were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which the aspects and embodiments described herein belong. The following references provide one of skill with a general definition of many of the terms used in the described aspects and embodiments: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a small compound, protein, nucleic acid molecule, or fragment thereof. In various embodiments, agents (e.g., phospho-specific antibodies, or antigen-binding fragments thereof, directed against a phosphorylated S104 residue of a CARD9 polypeptide, such as a CARD9 R101C variant polypeptide) that bind to this CARD9 variant polypeptide are provided. In various embodiments, anti-fungal agents (e.g., echinocandin; flucytosine; voriconazole; caspofungin; micafungin; posaconazole; isavuconazole; clotrimazole (Canesten); econazole; miconazole; terbinafine (Lamisil); fluconazole (Diflucan); ketoconazole (Daktarin); itraconazole; nystatin (Nystan); amphotericin (e.g., amphotericin B); and/or griseofulvin) are provided.

By “aggressive therapy” is meant the use of agents provided at a dosage or frequency that is higher than that typically used. Aggressive therapies are selected using methods described herein for subjects having or having a propensity to develop severe fungal infections.

By “alteration” is meant a change (increase or decrease) in the structure, expression levels or activity of a gene or polypeptide as detected by standard art known methods such as those described herein. As used herein, an alteration includes a 10% change in expression or activity levels, a 25% change, a 40% change, and a 50% or greater change in expression or activity levels. In some embodiments, an alteration in an anti-phospho-S104 CARD9 antibody is a sequence alteration that enhances binding to a target protein, stability, expression, function, or activity. In another embodiment, an alteration involves a decrease in the activity of CARD9, which may be associated with binding of a phospho-S104 CARD9 specific antibody.

By “ameliorate” is meant decrease, suppress, attenuate, diminish, arrest, or stabilize the development or progression of a disease, pathology, or condition. In some embodiments, diseases, pathologies, or conditions include those caused by or associated with fungal infections, e.g., without limitation, infection by C. albicans, C. auris, and T. rubrum, as well as other diseases that are associated with a CARD9 variant polypeptide described herein. In some embodiments, diseases, pathologies, or conditions include those caused or associated with abnormal CARD9 activation (e.g., increased activation of CARD9 in the subject as compared to a healthy subject), such as, but not limited to, Crohn's disease, inflammatory bowel disease, ankylosing sponditis, and/or IgA nephropathy.

By “analog” is meant a molecule that is not identical, but that has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. In addition, analogs of biparatopic antibodies that retain or enhance the activity of the original antibody are provided.

As used herein, the term “antibody” (Ab) refers to an immunoglobulin molecule that specifically binds to, or is immunologically reactive with, a particular antigen, and antigen binding fragments thereof. Exemplary antibodies encompass polyclonal, monoclonal, genetically and molecularly engineered and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (x) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)₂ fragments, that are capable of specifically binding to a target protein. Antibodies may include chimeric antibodies; recombinant and engineered antibodies, and antigen binding fragments thereof.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a VH domain; (vii) a dAb which consists of a VH or a VL domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CHI domains thereof, (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds). Without intending to be limiting, the antibodies described herein are monoclonal, phospho-specific antibodies that recognize and bind specifically to phosphorylated S104 (pS104) of the CARD9 polypeptide or peptide. In an embodiment, the anti-phospho-S104-specific antibody recognizes phosphorylation of S104 in the CARD9 protein independently of position R101. Thus, the antibody recognizes and binds to pS104 in the context of R101 (wildtype CARD9) or C101 (i.e., variant CARD9) in the CARD9 polypeptide. Exemplary anti-phospho-specific CARD9 S104 antibodies, which are defined herein, are useful in the methods in the various aspects and embodiments described herein.

As used herein, the term “complementarity determining region” (CDR) refers to a hypervariable region found both in the light chain and the heavy chain variable domains ((V_{L} and V_{H} domains, respectively). The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four framework regions (FR1, FR2, FR3, FR4) that primarily adopt a beta-sheet configuration, connected by three CDRs (CDR1, CDR2, CDR3), which form loops that connect, and in some cases form part of, the beta-sheet structure. The CDRs in each chain are held together in close proximity by the FR regions in the order FR1-CDR1-FR2-CDR2-FR3-CDR3-FR4. and the CDRs in each antibody chain contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

A “caspase recruitment domain 9 (“CARD9”) polypeptide” or “CARD9 protein” refers to a polypeptide, or fragment thereof, having at least about 85% amino acid sequence identity to the amino acid sequence of the CARD9 polypeptide (NCBI Reference Sequence: NP_434700.2) and having CARD modulating activity. A CARD9 polypeptide is a member of the caspase activation and recruitment domain (CARD) protein family, which is defined by the presence of a characteristic caspase activation and recruitment domain (CARD). CARD is a protein interaction domain known to participate in the activation or suppression of CARD-containing members of the caspase family, and thus plays an important regulatory role in cell apoptosis. The CARD9 protein was identified by its selective association with the CARD domain of BCL10, a positive regulator of apoptosis and NF-kappaB activation, and is thought to function as a molecular scaffold for the assembly of a BCL10 signaling complex that activates NF-kappaB. In embodiments, the polypeptide has at least about 90%, 93%, 95%, 98%, 99% or greater amino acid sequence identity to the amino acid sequence of the human CARD9 polypeptide (NCBI Reference Sequence: NP 434700.2). An exemplary human CARD9 polypeptide sequence is provided below (SEQ ID NO:1):

| | |
|---|---|
| 1 | MSDYENDDEC WSVLEGFRVT LTSVIDPSRI TPYLRQCKVL NPDDEEQVLS DPNLVIRKRK |
| 61 | VGVLLDILQR TGHKGYVAFL ESLELYYPQL YKKVTGKEPA RVFSMIIDAS GESGLTQLLM |
| 121 | TEVMKLQKKV QDLTALLSSK DDFIKELRVK DSLLRKHQER VQRLKEECEA GSRELKRCKE |
| 181 | ENYDLAMRLA HQSEEKGAAL MRNRDLQLEI DQLKHSLMKA EDDCKVERKH TLKLRHAMEQ |
| 241 | RPSQELLWEL QQEKALLQAR VQELEASVQE GKLDRSSPYI QVLEEDWRQA LRDHQEQANT |
| 301 | IFSLRKDLRQ GEARRLRCME EKEMFELQCL ALRKDSKMYK DRIEAILLQM EEVAIERDQA |
| 361 | IATREELHAQ HARGLQEKDA LRKQVRELGE KADELQLQVF QCEAQLLAVE GRLRRQQLET |
| 421 | LVLSSDLEDG SPRRSQELSL PQDLEDTQLS DKGCLAGGGS PKQPFAALHQ EQVLRNPHDA |
| 481 | GLSSGEPPEK ERRRLKESFE NYRRKRALRK MQKGWRQGEE DRENTTGSDN TDTEGS |

The sequence shown in bold is the RXXS motif.

An exemplary CARD9 polypeptide (protein) sequence of Mus musculus (NCBI Reference Sequence: NP_001032836.1) is provided below (SEQ ID NO: 2):

| | |
|---|---|
| 1 | MSDYENDDEC WSTLESFRVK LISVIDPSRI TPYLRQCKVL NPDDEEQVLS DPNLVIRKRK |
| 61 | VGVLLDILQR TGHKGYVAFL ESLELYYPQL YRKVTGKEPA RVFSMIIDAS GESGLTQLLM |
| 121 | TEVMKLQKKV QDLTALLSSK DDFIKELRVK DSLLRKHQER VQRLKEECEL SSAELKRCKD |
| 181 | ENYELAMCLA HLSEEKGAAL MRNRDLQLEV DRLRHSLMKA EDDCKVERKH TLKLRHAMEQ |
| 241 | RPSQELLWEL QQEKDLLQAR VQELQVSVQE GKLDRNSPYI QVLEEDWRQA LQEHQKQVST |
| 301 | IFSLRKDLRQ AETLRARCTE EKEMFELQCL ALRKDAKMYK DRIEAILLQM EEVSIERDQA |
| 361 | MASREELHAQ CTQSFQDKDK LRKLVRELGE KADELQLQLF QTESRLLAAE GRLKQQQLDM |
| 421 | LILSSDLEDS SPRNSQELSL PQDLEEDAQL SDKGVLADRE SPEQPFMALN KEHLSLTHGM |
| 481 | GPSSSEPPEK ERRRLKESFE NYRRKRALRK MQNSWRQGEG DRGNTTGSDN TDTEGS |

By “CARD9 polynucleotide” is meant a polynucleotide encoding a CARD9 polypeptide. An exemplary CARD9 polynucleotide sequence is provided at NCBI Reference Sequence: NM_052813.5, which is provided below (SEQ ID NO: 3):

| | |
|---|---|
| 1 | aagcagaacc catcaggaag tgcacaggcg tccggcgtgc tcctccctcc ctgcagcccc |
| 61 | gggcagcatc tcccagaggc tccgcggccc aggctcctgg tgtgtctgca gtgcaggtgg |
| 121 | ctcctggaag accctcagcc tgcctgctga ggccatgtcg gactacgaga acgatgacga |
| 181 | gtgctggagc gtcctggagg gcttccgggt gacgctcacc tcggtcatcg acccctcacg |
| 241 | catcacacct tacctgcggc agtgcaaggt cctgaacccc gatgatgagg agcaggtgct |
| 301 | cagcgacccc aacctggtca tccgcaaacg gaaagtgggt gtgctcctgg acatcctgca |
| 361 | gcggaccggc cacaagggct acgtggcctt cctcgagagc ctggagctct actacccgca |
| 421 | gctgtacaag aaggtcacag gcaaggagcc ggcccgcgtc ttctccatga tcatcgacgc |
| 481 | gtccggggag tcaggcctga ctcagctgct gatgactgag gtcatgaagc tgcagaagaa |
| 541 | ggtgcaggac ctgaccgcgc tgctgagctc caaagatgac ttcatcaagg agctgcgggt |
| 601 | gaaggacagc ctgctgcgca agcaccagga gcgtgtgcag aggctcaagg aggagtgcga |
| 661 | ggccggcagc cgcgagctca agcgctgcaa ggaggagaac tacgacctgg ccatgcgcct |
| 721 | ggcgcaccag agtgaggaga agggcgccgc gctcatgcgg aaccgtgacc tgcagctgga |
| 781 | gattgaccag ctcaagcaca gcctcatgaa ggccgaggac gactgcaagg tggagcgcaa |
| 841 | gcacacgctg aagctcaggc acgccatgga gcagcggccc agccaggagc tgctgtggga |
| 901 | gctgcagcag gagaaggccc tgctccaggc ccgggtgcag gagctggagg cctccgtcca |
| 961 | ggaggggaag ctggacagga gcagccccta catccaggta ctggaggagg actggcggca |
| 1021 | ggcgctgcgg gaccaccagg agcaggccaa caccatcttc tccctgcgca aggacctccg |
| 1081 | ccagggcgag gcccgacgcc tccggtgcat ggaggagaag gagatgttcg agctgcagtg |
| 1141 | cctggcacta cgtaaggact ccaagatgta caaggaccgc atcgaggcca tcctgctgca |
| 1201 | gatggaggag gtcgccattg agcgggacca ggccatagcc acgcgggagg agctgcacgc |
| 1261 | acagcacgcc cggggcctgc aggagaagga cgcgctgcgc aagcaggtgc gggagctggg |
| 1321 | cgagaaggcg gatgagctgc agctgcaggt gttccagtgt gaggcgcagc tactggccgt |
| 1381 | ggagggcagg ctcaggcggc agcagctgga gacgctcgtc ctgagctccg acctggaaga |
| 1441 | tggctcaccc aggaggtccc aggagctctc actcccccag gacctggagg acacccagct |
| 1501 | ctcagacaaa ggctgccttg ccggcggggg gagcccgaaa cagccctttg cagctctgca |
| 1561 | ccaggagcag gttttgcgga acccccatga cgcaggcctg agcagcgggg agccgcccga |
| 1621 | gaaggagcgg cggcgcctca aagagagttt tgagaactac cgcaggaagc gcgccctcag |
| 1681 | gaagatgcag aaaggatggc ggcaggggga ggaggaccgg gagaacacca cgggcagcga |
| 1741 | caacaccgac actgagggct cctagccgca gcagcgcagg ccccgaccag ggcacaccca |
| 1801 | ccggcccggc ctcctgccac ccgggggtgc cgacgccctg gggcgcagac ttccccgagc |
| 1861 | cgtcgctgac ttggcctgga acgaggaatc tggtgccctg aaaggcccag ccggactgcc |
| 1921 | gggcattggg gccgtttgtt aagcggcact cattttgcgg aggccatgcg ggtgctcacc |
| 1981 | acccccatgc acacgccatc tgtgtaactt caggatctgt tctgtttcac catgtaacac |
| 2041 | acaatacatg catgcattgt attagtgtta gaaaacacag ctgcgtaaat aaacagcacg |
| 2101 | ggtgacccgc a |

An exemplary CARD9 polynucleotide sequence of Mus musculus (NCBI Reference Sequence: NM_001037747.3) is provided below (SEQ ID NO: 4):

| | |
|---|---|
| 1 | ctccttcatg gctccaccct tctccagtta gggaacccct ccacactccc agagacccag |
| 61 | gctcctggta tgtccataac ccagacagca tctgctggca ggtagctctc acaagaccct |
| 121 | gagcctacag aggacatgtc agactatgag aatgacgacg agtgctggag caccctggag |
| 181 | agcttccggg tgaagctcat ctctgtcatt gacccctccc ggatcacacc ctatctacgc |
| 241 | cagtgcaaag tcctgaaccc cgatgatgag gagcaggtgc tcagtgaccc caacctggtc |
| 301 | atccgcaagc ggaaagtggg tgtgctcctg gacatcctgc agcggacagg ccacaagggc |
| 361 | tacgtggctt tcctcgagag cctggagctc tactaccctc agttataccg gaaagtcact |
| 421 | ggcaaggagc cagcacgcgt cttctccatg atcattgatg catcagggga gtctggcctg |
| 481 | acgcagctgc tgatgacaga ggtcatgaag ctgcagaaga aggttcagga cctgacggcc |
| 541 | cttctgagct ccaaggacga cttcatcaag gagctgaggg taaaggacag cctactgcgc |
| 601 | aagcaccagg agcgggtgca gcggctcaag gaggagtgtg agctgagcag tgcggagctg |
| 661 | aagcgctgca aggacgagaa ctatgagctg gccatgtgcc tggcacatct gagtgaagag |
| 721 | aagggcgcag cactcatgcg gaaccgtgac ctgcagcttg aggtggaccg gctcaggcac |
| 781 | agcctcatga aggccgagga tgactgcaag gtggagcgca aacacacact gaagctcagg |
| 841 | cacgccatgg agcagcggcc tagtcaggag ctgctgtggg aactacagca ggaaaaggac |
| 901 | ttgctgcagg cccgggtgca ggagctgcag gtctctgtgc aggagggtaa gctagacagg |
| 961 | aatagtccat acattcaagt gctggaggag gactggcgtc aagcactgca ggaacaccag |
| 1021 | aagcaggtca gcaccatctt ctccctacgg aaggacctcc gccaggctga gaccctccgg |
| 1081 | gcccggtgca cggaagaaaa ggagatgttc gagctgcagt gcctggcctt gcgcaaggat |
| 1141 | gccaagatgt acaaggaccg tatcgaggct atcctgctgc agatggagga ggtctccatt |
| 1201 | gagagggacc aggctatggc ctccagggaa gagctgcatg cacagtgtac ccaaagcttt |
| 1261 | caggacaaag ataagcttcg aaagctggtt cgagagctgg gtgagaaggc agatgagctg |
| 1321 | cagatacagc tgttccagac ggagagccga ttactggccg ccgagggcag actcaagcag |
| 1381 | cagcaattgg acatgctcat cctgagctct gacttggaag acagttcacc caggaactcc |
| 1441 | caggagctct cactgcctca ggatctggag gaggatgccc agctctcaga caaaggtgta |
| 1501 | ctggcagaca gggagagccc agagcagccc tttatggctc tgaacaagga gcatctttca |
| 1561 | ctgacccatg gcatggggcc cagcagcagc gagcccccgg agaaggagcg gcggcgcctc |
| 1621 | aaggagagct tcgagaacta ccgcaggaag cgggcgctcc gcaagatgca gaacagctgg |
| 1681 | cggcagggag aaggggatcg cgggaatacg acaggcagcg acaacaccga caccgagggc |
| 1741 | tcctagcgaa ccgcgccgag gctgagcatc tgtggaattg tgaaaggatg ctgcggtttt |
| 1801 | tttttttttt tttttttttt tactgtatta gaattagaaa atgcaactaa ataaaataat |
| 1861 | caccgagctg a |

In embodiments, a CARD9 polynucleotide has at least about 90%, 93%, 95%, 98%, 99% or greater nucleic acid sequence identity to a CARD9 polynucleotide.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. In various aspects and embodiments, antibodies comprising modifications in these hybrid hypervariable positions are provided. The variable domains of native heavy and light chains each comprise four framework regions that primarily adopt a beta-sheet configuration, connected by three CDRs, which form loops that connect, and in some cases form part of, the .beta.-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

The term “variable region CDR” includes amino acids in a CDR or complementarity determining region as identified using sequence or structure based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. In certain embodiments, the term “CDR” is a CDR as defined by Kabat based on sequence comparisons.

“Detect” refers to identifying the presence, absence or amount of the analyte to be detected. In some embodiments, the analyte is an antigen, epitope, or fragment thereof. In one embodiment, the term “detect” refers to detecting antibody binding to an agent of interest. In some embodiments, the analyte is a CARD9 polypeptide (e.g., phosphorylated or non-phosphorylated) or fragment thereof.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. In some embodiments, an antibody as described herein is directly or indirectly linked to a detectable label.

By “disease” is meant any condition or disorder that damages or interferes with the normal function of a cell, tissue, or organ. Examples of diseases, disorders, pathologies, or conditions that may be characterized through the use of the products, compositions and methods herein include those associated with fungal infections. In some embodiments, diseases include those caused or associated with abnormal CARD9 activation (e.g., increased activation of CARD9 in the subject as compared to a healthy subject), such as, but not limited to, Crohn's disease, inflammatory bowel disease, ankylosing sponditis, and/or IgA nephropathy.

By “effective amount” is meant the amount of an agent required to ameliorate the symptoms of a disease relative to an untreated patient. The effective amount of active compound(s) used to practice methods for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, antibodies described herein are used to identify subjects at risk of developing severe fungal infections, and selecting an appropriately aggressive therapy for such subjects. In some embodiments, antibodies described herein are used to identify subjects having, or at risk of having a disease associated with abnormal CARD9 activation (e.g., increased activation of CARD9 in the subject as compared to a healthy subject), such as, but not limited to, Crohn's disease, inflammatory bowel disease, ankylosing sponditis, and/or IgA nephropathy.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “fusion protein” or simply “fusion” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., 2012, Bioorg. Med. Chem., 20:571-582).

As used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell (e.g., yeast) that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and 6,180,370 to Queen et al; EP239400; PCT publication WO 91/09967; U.S. Pat. No. 5,225,539; EP592106; and EP519596; incorporated herein by reference.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of some aspects and embodiments is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of some aspects and embodiments herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of some aspects and embodiments that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of some aspects and embodiments herein. An isolated polypeptide of some aspects and embodiments herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, the term “operatively linked” in the context of a polynucleotide fragment is intended to mean that the two polynucleotide fragments are joined such that the amino acid sequences encoded by the two polynucleotide fragments remain in-frame.

By “reduces” or “reduction” is meant a negative alteration of at least 10%, 25%, 50%, 75%, or 100%.

By “reference” is meant a standard or control condition. In some embodiments, a cell having a CARD9 mutation is contacted by an antibody described herein to detect the presence and/or levels of an antigen, e.g., a phosphorylated S104 residue in a CARD9 polypeptide, in the cell, and the alteration in the presence and/or levels of the antigen is determined relative to a corresponding reference cell not having the CARD9 mutation. In some embodiments, the reference is the proliferation, cell survival, or cell death observed in the control cell. In some embodiments, the reference is a reference subject not having a CARD9 mutation (e.g., a CARD9 mutation that prevents phosphorylation of S104, such as R101C).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

A “sample” or “biological sample” refers to specimen obtained, taken, generated, or derived from a subject or individual, such as a patient. The specimen may be a body fluid, such as blood, plasma, serum, saliva, sputum, tears, urine; other body fluids, e.g., bronchial fluid, lavage fluid, CNS fluid; stool; cells; tissues; organs (e.g., spleen); and the like. In an embodiment, the sample is a cell sample, e.g., a sample of cells from a site of fungal infection. In an embodiment, the cell is a bone marrow cell, a bone marrow derived cell, a stem cell, or a progenitor cell. In an embodiment, the cell is derived from blood or bone marrow. In an embodiment, the cell is a human cell. In embodiments, the cells are primary cells or are cultured cells.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of some aspects and embodiments herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively, or in addition, mutations are made to CDR amino acid residues to optimize antigen binding using art recognized techniques. scFv fragments are described, for example, in WO 2011/084714; incorporated herein by reference.

By “specifically binds” is meant a polypeptide or antibody that recognizes and binds a polypeptide of interest (e.g., phospho-S104 of CARD9), but which does not substantially recognize and bind other molecules in a sample, for example, a biological sample, which naturally includes a polypeptide of some aspects and embodiments herein. An antibody or antigen-binding fragment thereof that specifically binds to an antigen will bind to the antigen with a KD of less than 100 nM. For example, an antibody or antigen-binding fragment thereof that specifically binds to an antigen will bind to the antigen with a KD of up to 100 nM (e.g., between 1 pM and 100 nM). An antibody or antigen-binding fragment thereof that does not exhibit specific binding to a particular antigen or epitope thereof will exhibit a KD of greater than 100 nM (e.g., greater than 500 nm, 1 uM, 100 uM, 500 uM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of some aspects and embodiments herein include any nucleic acid molecule that encodes a polypeptide of some aspects and embodiments herein or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of some aspects and embodiments herein include any nucleic acid molecule that encodes a polypeptide of some aspects and embodiments herein, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 370 C, and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 370 C in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 250 C, more preferably of at least about 420 C, and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 680 C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline mammal. Other mammals include, without limitation, non-human primates (monkeys and the like), mice, rats, rabbits, guinea pigs, gerbils, llamas and alpacas.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “transfecting” or “transfection” is used synonymously and according to some aspects and embodiments herein means the introduction of heterologous nucleic acid (DNA/RNA) into a eukaryotic cell, in particular yeast cells.

According to some aspects and embodiments herein, antibody fragments are understood as meaning functional parts of antibodies, such as Fc, Fab, Fab′, Fv, F(ab′)2, scFv. According to some aspects and embodiments herein, corresponding biological active fragments are to be understood as meaning those parts of antibodies which are capable of binding to an antigen, such as Fab, Fab′, Fv, F(ab′)2, and scFv.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. In embodiments, a subject having a propensity to develop a severe fungal infection is identified using the antibodies described herein. Such subjects are selected for aggressive treatment.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors of some aspects and embodiments herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

Unless specifically stated or obvious from context, as used herein, the term “or” is understood to be inclusive. Unless specifically stated or obvious from context, as used herein, the terms “a”, “an”, and “the” are understood to be singular or plural.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold, of a value. Where particular values are described in the application and claims, unless otherwise stated the term “about” meaning within an acceptable error range for the particular value should be assumed.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E provide schematics, alignments, graphs, and pictures showing that CARD9 linker residues R101 and S104 are required to elicit cytokine responses. FIG. 1A is a schematic representation of known CARD9 genetic risk variants associated with susceptibility to fungal infection. Linker region is shown. FIG. 1B, is an alignment of the linker region of CARD9 and CARD11 in higher vertebrates. Figure discloses SEQ ID NOS 61-68, respectively, in order of appearance. FIG. 1C is a schematic representation of CARD9 2-142 structure (PBD ID: 6N2M), projecting R101 and S104 location in the linker region that was visualized using PyMOL 2.4.0 (Schrödinger, LLC). FIG. 1D is a graph showing TNFα concentration in the supernatant of CARD9−/− murine BMDCs transduced with lentiviruses containing Empty vector (FS), CARD9 FL, CARD9 R101C or CARD9 S104N and stimulated with HKCA (MOI 1:10), HKTR (MOI 1:10), WGP (50 μg/ml) or LPS (10 ng/ml) for 24h. (n=3 mice per condition). The bars in the graph are shown in groups of 4 (from left to right, FS, CARD9 WT, CARD9 R101C, CARD9 S104N) for each different condition (i.e., unstimulated, HKCA, HKTR, WGP, or LPS). FIG. 1E shows expression of CARD9 in the lysates from panel b determined by western blot (WB) with the indicated antibodies. Each experiment was repeated 3 times. Error Bars represent mean+/−SEM. *p<0.05, ** p<0.01 from paired t-test.

FIGS. 2A-2E provide pictures and graphs showing that CARD9 activation through S104 phosphorylation is impaired by the R101C variant implicated in fungal disease. FIG. 2A is a picture showing CARD9 immunoprecipitates from BMDCs from WT, CARD9−/− or CARD9 R101C mutant treated with HKCA (MOI 1:10) for 30 minutes. FIG. 2B is a picture showing Bcl10 immunoprecipitates from BMDCs from WT, CARD9−/− or CARD9 R101C mutant treated with HKCA (MOI 1:10) for 30 minutes. FIG. 2C is a picture showing WB of BMDCs from WT, CARD9−/− or CARD9 R101C mutant treated with HKCA (MOI 1:10), HKTR (MOI 1:10) or WGP (50 ug/ml) for 15 minutes. FIG. 2D is a graph showing Nuclear p65 translocation in BMDCs from WT, CARD9−/− or CARD9 R101C mutant treated with indicated doses of WGP, HKCA, or HKTR for 30 min. The bars in the graph are shown in groups of 3 (from left to right, WT, CARD9−/−, CARD9 R101C) for each different condition (i.e., unstimulated, HKCA 1:1, HKCA 1:5, HKTR 1:1, HKTR 1:5, HKTR 1:10, WGP 10 μg/ml, WGP 25 μg/ml, or WGP 50 μg/ml). FIG. 2E is a graph showing TNFα concentration in the supernatant from BMDCs from WT, CARD9−/− or CARD9 R101C mutant treated with indicated doses of WGP, HKCA, HKTR or LPS (10 ng/ml) for 24h. (n=3 mice per condition). The bars in the graph are shown in groups of 3 (from left to right, WT, CARD9−/−, CARD9 R101C) for each different condition (i.e., unstimulated, HKCA 1:1, HKCA 1:5, HKTR 1:1, HKTR 1:5, HKTR 1:10, WGP 10 μg/ml, WGP 25 μg/ml, or WGP 50 μg/ml). Each experiment was repeated 3 times. Bars represent mean+/−SEM.

FIGS. 3A-3H provide graphs and pictures showing that CARD9 R101 functions as a signaling switch activated by S104 phosphorylation. FIG. 3A, BMDCs expressing Cas9 were transduced with guides for the corresponding genes on day 2 and selected with puromycin. On day 9 cells were stimulated with 100 ug/ml WGP O/N and IL-6 levels in supernatant were measured with ELISA. FIG. 3B, BMDCs expressing Cas9 were transduced with guides for the corresponding genes on day 2 and selected with puromycin. On day 9 cells were stimulated with HKCA (MOI 1:10), HKTR (1:10) or LPS (10 ng/ml) for 30 min. p65 nuclear translocation was measured using fluorescence imaging. The bars in the graph are shown in groups of 6 (from left to right, EGFP, EGFP, PAK2, PAK2, PKCd, PKCd) for each different condition (i.e., untreated, HKCA, HKTR, LPS). FIG. 3C, CARD9 FL, CARD9 R101C or CARD9 S104N were overexpressed in HEK 293T cells. Following anti-flag pull-down, beads were incubated with purified PKCd in presence or absence of ATP and processed for WB with the indicated antibodies. FIG. 3D, BMDCs from WT mice were treated with HKCA (MOI 1:10) or HKCA (MOI 1:10) with PKCδi (Sotrastaurin) for 15 minutes and processed for immunoprecipitation with anti-CARD9 antibody. Samples were analyzed by WB with the indicated antibodies. FIG. 3E, Control or PKCδ KO BMDCs were treated with HKCA (MOI 1:10) for 15 minutes and processed for immunoprecipitation with anti-CARD9 antibody. Samples were analyzed by WB with the indicated antibodies. FIG. 3F, Normalized fluorescence polarization representing filament formation between different CARD9 2-152 variants and Bcl10, average of quadruplicates. The graph lines are, in order from top to bottom, 1:1 MBP-Bcl10+CARD9 2-152/I107E (1 mM Incubation)+TEV, 1:1 MBP-Bcl10+CARD9 2-152/S104D (2 mM Incubation)+TEV, 1:1 MBP-Bcl10+CARD9 2-152/R101C/S104D (2 mM Incubation)+TEV, 1:1 MBP-Bcl10+CARD9 2-152/WT (2 mM Incubation)+TEV, 1:1 MBP-Bcl10+CARD9 2-152/R101C (2 mM Incubation)+TEV. FIG. 3G, TNFα concentration in the supernatant in CARD9−/− murine BMDCs transduced with lentiviruses containing empty vector (FS), CARD9 FL in three different concentrations, CARD9 S104D or CARD9 S104D/R101C and stimulated with HKCA (MOI 1:10), HKTR (MOI 1:10), WGP (50 μg/ml) or LPS (10 ng/ml) for 24 h. The bars in the graph are shown in groups of 6 (from left to right, FS, CARD9 WT, CARD9 WT 1:2, CARD9 WT 1:4, CARD9 S104D, CARD9 S104D/R101C) for each different condition (i.e., unstimulated, HKCA, HKTR, WGP, or LPS). FIG. 3H, Expression of CARD9 in the lysates from panel g determined by western blot (WB) with the indicated antibodies. Each experiment was repeated 3 times. Error Bars represent mean+/−SEM. *p<0.05, ** p<0.01, *** p<0.001, ****p<0.00001 from paired t-test.

FIGS. 4A-4K provide graphs and pictures showing that CARD9 R101C mice are predisposed to systemic fungal infection. FIG. 4A, survivorship of Mice injected IV with live C. albicans over time (n=5 for each genotype). The graph lines are, in order from left to right, WT, CARD9−/−, CARD9 R101C. FIG. 4B, CFU from the kidney or brain of mice injected IV with 10\hat{ }5 live C. albicans and analyzed at day 2 post injection (n=4 for each genotype). FIG. 4C, representative PAS staining of kidneys from panel b. FIG. 4D, quantification of FIG. 4C. FIG. 4E, representative PAS staining of brains from FIG. 4B. FIG. 4F, quantification of FIG. 4E. FIG. 4G, flow cytometry analysis of neutrophil composition in the kidneys from FIG. 4B. FIG. 4H, flow cytometry analysis of inflammatory monocytes in the kidneys from FIG. 4B. FIG. 4I, flow cytometry analysis of neutrophil composition in the brains from FIG. 4B. FIG. 4J, flow cytometry analysis of inflammatory monocytes in the brains from FIG. 4B. FIG. 4K, serum and kidney IL-6 levels in mice from FIG. 4B. The bars in the graph are shown in groups of 3 (from left to right, WT, CARD9−/−, CARD9 R101C) for each different condition (i.e., serum or kidney). Each experiment was repeated at least 2 times. Error Bars represent mean+/−SEM. * p<0.05, ** p<0.01,*** p<0.001, **** p<0.0001 from paired t-test. Scale bars represent 1000 μm or 100 μm.

FIGS. 5A-5L provide graphs and pictures showing that CARD9 R101C mutation impairs spore clearance in a mouse model of dermatophytosis. FIG. 5A, CFU from back skin of mice injected intradermally with 10\hat{ }5 live T. rubrum and analyzed at day 2 post injection (n=4 for each genotype). FIG. 5B, representative H&E staining of back skin from FIG. 5A. FIG. 5C, flow cytometry analysis of total CD45 positive cells composition in the back skin from FIG. 5A. FIG. 5D, flow cytometry analysis of monocytes composition in the back skin from FIG. 5A. FIG. 5E, flow cytometry analysis of neutrophils composition in the back skin from FIG. 5A. FIG. 5F, CXCL1 concentration in the back skin from panel a measured by ELISA. FIG. 5G, CFU from the back skin of mice injected intradermally with 10\hat{ }5 live T. rubrum and analyzed at day 9 post injection (n=4 for each genotype). FIG. 5H, representative H&E staining of back skin from FIG. 5G. FIG. 5I, flow cytometry analysis of total CD45 positive cells composition in the back skin from FIG. 5G. FIG. 5J, flow cytometry analysis of monocytes composition in the back skin from FIG. 5G. FIG. 5K, flow cytometry analysis of neutrophils composition in the back skin from FIG. 5G. FIG. 5L, CXCL1 concentration in the back skin from FIG. 5G measured by ELISA. Each experiment was repeated at least 2 times. Scale bars represents 100 μm (panels b and h). Error Bars represent mean+/−SEM. * p<0.05, ** p<0.01 from paired t-test.

FIGS. 6A-6E provide a cluster map, graphs, and charts showing that CARD9 R101C impairs inflammatory signaling pathways and cell-cell communication circuitry in skin immune, stromal, and epithelial cells. FIG. 6A, clustering of cells based on expression obtained after scRNAseq of CARD9 WT and CARD9 R101C skin infected with T. rubrum and analyzed 2 and 9 days post infection. n=6 for CARD9 WT and n=6 for CARD9 R101C (3 replicates of each WT and CARD9 R101C at each timepoint). FIG. 6B, prevalence of each immune cluster across CARD9 genotypes and timepoints. Bar plots show the mean prevalence of each cluster across 3 replicates. For each cluster, 4 comparisons are conducted: D2 WT vs D2 R101C; D9 WT vs D9 R101C; D2 WT vs D9 WT; D2 ROIC vs D9 R101C. The bars in the graph are shown in groups of 4 (from left to right, D2 WT, D2 R101C, D9 WT, D9 R101C) for each different cluster (i.e., Mono1, Mono2, Mac1, DC, Mac2, Mac3, L-hans, Th17, Mast). Comparisons were conducted using Dirichlet multinomial regression. Comparisons were corrected for multiple testing using the Benjamini-Hochberg method; stars indicate comparisons significant at FDR P<0.1. FIG. 6C, top 20 significantly differentially expressed genes (DEGs) between D2 WT and D2 R101C cells within the Langerhans cell cluster. Scale bar indicates z-score. Significance was defined as FDR P<0.05. FIG. 6D, top pathways enriched in immune clusters based on DEGs between D2 WT cells and D2 R101C cells within each immune cluster. NES indicates normalized enrichment score from gene set enrichment analysis (GSEA) using DEGs between D2 WT cells and D2 R101C cells within each cluster; NES <0 indicates upregulated in WT, NES>0 indicates upregulated in R101C. FIG. 6E, Mean expression of cytokines, chemokines, effector molecules (top dot plot), and their corresponding receptors (bottom dot plot) in each cluster. For each molecule, the mean expression across all cells in each cluster regardless of Card9 genotype is shown. Increasing dot size indicates higher expression of the marker in the corresponding cluster. Colors indicate whether the molecule is significantly upregulated in D2 WT cells relative to D2 R101C cells in the corresponding cluster (beige), D2 R101C cells relative to D2 WT cells in the corresponding cluster (green), or not differentially expressed between D2 WT and D2 R101C cells in the corresponding cluster (white).

FIGS. 7A-7L provide heat maps and graphs showing cell non-autonomous effects of CARD9 R101C. FIG. 7A, heatmap depicts top 20 differentially expressed genes (DEGs) between D2 WT cells and D2 R101C cells in fibroblasts. Row annotations show whether the gene is significantly differentially expressed in D9 WT vs R101C comparison, WT D2 vs D9 comparison, and R101C D2 vs D9 comparison in fibroblasts. FIG. 7B, heatmap depicts top 20 DEGs between D2 WT and D2 R101C cells in endothelial cells. Row annotations show whether the gene is significantly differentially expressed in D9 WT vs R101C comparison, WT D2 vs D9 comparison, and R101C D2 vs D9 comparison in endothelial cells. FIG. 7C, heatmap depicts top 20 DEGs between D2 WT and D2 R101C cells in the Kera differentiated clusters. Row annotations show differential expression of the corresponding gene for the D9 WT vs R101C comparison, WT D2 vs D9 comparison, and R101C D2 vs D9 comparison. FIG. 7D, top pathways enriched in stromal and epithelial clusters based on differentially expressed genes (DEGs) between D2 WT cells and D2 R101C cells within each stromal and epithelial cluster. NES indicates normalized enrichment score from gene set enrichment analysis (GSEA); NES <0 indicates upregulated in WT, NES>0 indicates upregulated in R101C. FIGS. 7E-7H, changes in keratinocyte cluster frequency between D2 WT and D2 R101C. Specifically, plots show proportion of all cells at D2 in each sample for keratinocyte clusters with significantly different (FDR P<0.1; Dirichlet multinomial regression) prevalence in WT and R101C cells at D2. FIGS. 7I-7L, changes in keratinocyte cluster frequencies between D2 and D9 and between CARD9 genotypes. Specifically, plots show proportion of all cells at the indicated timepoint in each sample for keratinocyte clusters with significantly different (FDR P<0.1; Dirichlet multinomial regression) prevalence in WT and R101C cells, or between D2 and D9. Plots are shown for significant comparisons.

FIGS. 8A-8C provide graphs and a picture showing that CARD9 linker residues R101 and S104 are required to elicit cytokine responses FIG. 8A, IL-6 concentration in the supernatants from CARD9−/− murine BMDCs transduced with lentiviruses containing Empty vector (FS), CARD9 FL, CARD9 R101C or CARD9 S104N and stimulated with HKCA (MOI 1:10), HKTR (MOI 1:10), WGP (50 μg/ml) or LPS (10 ng/ml) for 24h. (n=3 mice per condition). The bars in the graph are shown in groups of 4 (from left to right, FS, CARD9 WT, CARD9 R101C, CARD9 S104N) for each different condition (i.e., unstimulated, HKCA, HKTR, WGP, LPS). FIG. 8B, TNFa concentration in the supernatant from CARD9−/− murine BMDMs transduced with lentiviruses containing Empty vector (FS), CARD9 FL, CARD9 FL diluted 1:2, CARD9 FL diluted 1:4, CARD9 R101C, CARD9 S104N, CARD9 S104D or CARD9 S104D/R101C and stimulated with HKCA (MOI 1:10), HKTR (MOI 1:10) or LPS (10 ng/ml) for 24h. (n=3 mice per condition). The bars in the graph are shown in groups of 8 (from left to right, FS, CARD9 WT, CARD9 WT (1:2), CARD9 WT (1:4), CARD9 R101C, CARD9 S104N, CARD9 S104D, CARD9 R101C/S104D) for each different condition (i.e., unstimulated, HKCA, HKTR, LPS). FIG. 8C, expression of CARD9 in the lysates from b determined by western blot (WB) with the indicated antibodies. Each experiment was repeated 3 times. Error Bars represent mean+/−SEM. *p<0.05, ** p<0.01, *** p<0.001 from paired t-test.

FIGS. 9A-9J provide schematics, pictures, a table, graphs showing that CARD9 activation through S104 phosphorylation is impaired by the R101C variant implicated in fungal disease. FIG. 9A, schematic representation of CRISPR KI into CARD9 allele to obtain endogenous CARD9 R101C mutation. Figure discloses SEQ ID NOS 69-71, respectively, in order of appearance. FIG. 9B, representative sequencing result highlighting the presence of mutated base pair (arrow). Figure discloses SEQ ID NOS 72-74, respectively, in order of appearance. FIG. 9C, WB from BMDCs isolated from WT or CARD9 R101C mutant mice. FIG. 9D, WB from splenic CD11c positive cells isolated from WT or CARD9 R101C mutant mice. FIG. 9E, table summarizing hybridoma screening results for binding affinity of different phospho-S104 specific antibodies. ELISA results are shown as values of absorbance at 490 nM, representing antibody reactivity towards either pS104, or pS104/R101C, or S104 peptides. Antibody used in the study is row 6 (i.e., 4F9.H5). FIG. 9F, IL-6 concentration in supernatants from BMDCs from WT, CARD9−/− or CARD9R101C mutant treated with indicated concentrations of WGP, HKCA, HKTR or LPS (10 ng/ml) for 24h. (n=3 mice per condition). The bars in the graph are shown in groups of 3 (from left to right, WT, CARD9−/−, CARD9 R101C) for each different condition (i.e., untreated, HKCA 1:1, HKCA 1:5, HKCA 1:10, HKTR 1:1, HKTR 1:5, HKTR 1:10, WGP 10 μg/ml, WGP 25 μg/ml, WGP 50 μg/ml, or LPS). FIG. 9G, TNFa concentration in BMDMs from WT, CARD9−/− or CARD9R101C mutant treated with WGP (50 ug/ml), HKCA(MOI 1:10), HKTR (MOI 1:10) or LPS (10 ng/ml) for 24 h. The bars in the graph are shown in groups of 3 (from left to right, WT, CARD9−/−, CARD9 R101C) for each different condition (i.e., untreated, HKCA 1:10, HKTR, WGP, LPS). FIG. 9H, Nuclear p65 translocation in BMDMs from WT, CARD9−/− or CARD9R101C mutant treated with HKCA(MOI 1:10), HKTR (MOI 1:10) or LPS (10 ng/ml) for 30 min. The bars in the graph are shown in groups of 3 (from left to right, WT, CARD9−/−, CARD9 R101C) for each different condition (i.e., untreated, HKCA, HKTR, LPS). FIG. 9I, IL-6 concentration in supernatant from bone marrow monocytes from WT, CARD9−/− or CARD9R101C mutant treated with WGP (50 ug/ml), HKCA(MOI 1:10), HKTR (MOI 1:10) or LPS (10 ng/ml) for 24 h. The bars in the graph are shown in groups of 3 (from left to right, WT, CARD9−/−, CARD9 R101C) for each different condition (i.e., untreated, HKCA, HKTR, WGP, LPS). FIG. 9J, TNFa concentration in supernatant from bone marrow monocytes from WT, CARD9−/− or CARD9 R101C mutant treated with WGP (50 ug/ml), HKCA(MOI 1:10), HKTR (MOI 1:10) or LPS (10 ng/ml) for 24 h. The bars in the graph are shown in groups of 3 (from left to right, WT, CARD9−/−, CARD9 R101C) for each different condition (i.e., untreated, HKCA, HKTR, WGP, LPS). Each experiment was repeated 3 times. Bars represent mean+/−SEM. * p<0.05, ** p<0.01, **** p<0.0001 from paired t-test.

FIGS. 10A-10F provide graphs and pictures showing that CARD9 R101 functions as a signaling switch activated by S104 phosphorylation. FIG. 10A, supernatant IL-6 concentration in BMDCs expressing Cas9 transduced with guides for the corresponding genes on day 2 and selected with puromycin. On day 9 cells were stimulated with HKCA (MOI 1:10), HKTR (1:10) or WGP (50 ug/ml) O/N. The bars in the graph are shown in groups of 6 (from left to right, EGFP, EGFP, PAK2, PAK2, PKCd, PKCd) for each different condition (i.e., untreated, HKCA, HKTR, WGP). FIG. 10B, IL-6 concentration in BMDCs from WT mice treated with HKCA (MOI 1:10) or HKCA (MOI 1:10) with PKCdi (Sotrastaurin) O/N. The bars in the graph are shown in groups of 3 (from left to right, WT, WT+PKCdi, CARD9−/−) for each different condition (i.e., untreated, HKCA, HKTR, WGP). FIG. 10C, TNFa concentration in BMDCs from WT mice treated with HKCA (MOI 1:10) or HKCA (MOI 1:10) with PKCdi (Sotrastaurin) O/N. The bars in the graph are shown in groups of 3 (from left to right, WT, WT+PKCdi, CARD9−/−) for each different condition (i.e., untreated, HKCA, HKTR, WGP). FIG. 10D, WB representing BL21 DE3 expression of CARD9 2-152 and MBP-Bcl10 at corresponding time points. Each time point shows expression from the soluble fraction (S) and insoluble fraction (In). FIG. 10E, normalized fluorescence polarization representing filament formation between different CARD9 2-152 variants and Bcl10, average of quadruplicates. The lines in the graph, from top to bottom, are 1:1 CARD9 2-152/R101C/1107E (2 mM Incubation)+TEV, 1:1 CARD9 2-152/1107E (1 mM Incubation+TEV), and 1:1 CARD9 2-152/R101C (2 mM Incubation)+TEV. FIG. 10F, IL-6 concentration in supernatants from CARD9−/− murine BMDCs transduced with lentiviruses containing empty vector (ES), CARD9 FL in three different concentrations, CARD9 S104D or CARD9 S104D/R101C and stimulated with HKCA (MOI 1:10), HKTR (MOI 1:10), WGP (50 μg/ml) or LPS (10 ng/ml) for 24h. (n=3 mice per condition). The bars in the graph are shown in groups of 6 (from left to right, FS, CARD9 WT, CARD9 WT 1:2, CARD9 WT 1:4, CARD9 S104D, CARD9 S104D/R101C) for each different condition (i.e., unstimulated, HKCA, HKTR, WGP, LPS). Error bars represent mean+/−SEM. **p<0.01, *** p<0.001 from paired t-test.

FIGS. 11A-11H provide graphs showing that CARD9 R101C mice have normal immune cell composition in steady state. FIG. 11A, flow cytometry analysis of T-cell composition in the spleen from 12-week old CARD9 mice (n=4 for each genotype). The bars in the graph are shown in groups of 3 (from left to right, WT, CARD9−/−, CARD9 R101C) for each different cell type (i.e., CD3+ total T cells, CD4+ T cells, CD8+ T cells, CD8+ CD122−, CD8+ CD122+). FIG. 11B, flow cytometry analysis of B-cell composition in the spleen from 12-week old CARD9 mice (n=4 for each genotype). Total B cells (B220+), Marginal Zone B cells (B220+, CD21/35 hi, CD23low), Follicular B cells (B220+, CD23 hi, CD21/35int). The bars in the graph are shown in groups of 3 (from left to right, WT, CARD9−/−, CARD9 R101C) for each different cell type (i.e., Total B cells, Folicular B cells, Marginal Zone B cells). FIG. 11C, flow cytometry analysis of myeloid cells composition in the spleen from 12-week old CARD9 mice (n=4 for each genotype). The bars in the graph are shown in groups of 3 (from left to right, WT, CARD9−/−, CARD9 R101C) for each different cell type (i.e., Neutrophils, Macrophages, Dendritic cells). FIG. 11D, flow cytometry analysis of naïve T-cell composition in the thymus from 12-week old CARD9 mice (n=4 for each genotype). DN1 (CD44+, CD25−), DN2 (CD44+, CD25+), DN3 (CD44−, CD25+), DN4 (CD44−, CD25−). The bars in the graph are shown in groups of 3 (from left to right, WT, CARD9−/−, CARD9 R101C) for each different cell type (i.e., DN1, DN2, DN3, DN4). FIG. 11E, flow cytometry analysis of early T-cell progenitor (ETP) composition in the thymus from 12-week old CARD9 mice (n=4 for each genotype). ETP (CD44+, CD25−, cKit+). FIG. 11F, flow cytometry analysis of T-cell composition in the thymus from 12-week old CARD9 mice (n=4 for each genotype). DP (CD4+, CD8+), DN (CD4−, CD8−), SP4 (CD4+, CD8−), SP8 (CD4−, CD8+). The bars in the graph are shown in groups of 3 (from left to right, WT, CARD9−/−, CARD9 R101C) for each different cell type (i.e., DP, DN, SP4, SP8). FIG. 11G, flow cytometry analysis of bone marrow Hardy fraction D-F from 12-week old CARD9 mice (n=4 for each genotype). Fr. D (B220+ CD43−), Fr. E (B220+, CD43−, IgM+), Fr. F (B220+, CD43−, IgM+, IgD+). The bars in the graph are shown in groups of 3 (from left to right, WT, CARD9−/−, CARD9 R101C) for each different cell type (i.e., Fr. D, Fr. E, Fr. F). FIG. 11H, Flow cytometry analysis of bone marrow Hardy fraction A-C from 12-week old CARD9 mice (n=4 for each genotype). Fr. A (B220+, CD43+, BP-1−, CD24−), Fr. B (B220+, CD43−, CD24+, BP-1−), Fr. C (CD22+, CD43+, CD24+, BP-1+). The bars in the graph are shown in groups of 3 (from left to right, WT, CARD9−/−, CARD9 R101C) for each different cell type (i.e., Hardy Fr. A, Hardy Fr. B, Hardy Fr. C). Error bars represent mean+/−SEM.

FIGS. 12A-12E provide schematics and graphs showing that CARD9 R101C mice are predisposed to systemic fungal infection. FIG. 12A, gating strategy applied to analyze kidney samples from FIGS. 4A-4K and FIGS. 12B-12D. FIG. 12B, flow cytometry analysis of macrophage composition in the kidney from mice injected IV with 10\hat{ }5 live C. albicans and analyzed at day 2 post injection (n=4 for each genotype). FIG. 12C, flow cytometry analysis of dendritic cell composition in the kidney from FIG. 12B. FIG. 12D, flow cytometry analysis of eosinophil composition in the kidney from FIG. 12B. FIG. 12E, gating strategy applied to analyze brain samples from FIGS. 4A-4K and FIG. 12B-12D. Each experiment was repeated at least 2 times. Bars represent mean+/−SEM.

FIGS. 13A-13J provide graphs and schematics showing that CARD9 R101C mice are predisposed to systemic fungal infection. FIG. 13A, flow cytometry analysis of macrophage composition in the brain from mice injected IV with 10\hat{ }5 live C. albicans and analyzed at day 2 post injection (n=4 for each genotype). FIG. 13B, flow cytometry analysis of dendritic cell composition in the brain from FIG. 13A. FIG. 13C, flow cytometry analysis of eosinophil composition in the kidney from FIG. 13A. FIG. 13D, gating strategy applied to analyze spleen samples from FIGS. 13A-13C and 13E-13J. FIG. 13E, flow cytometry analysis of macrophage composition in the spleen from mice injected IV with 10\hat{ }5 live C. albicans and analyzed at day 2 post injection (n=4 for each genotype). FIG. 13F, flow cytometry analysis of total dendritic cell in the spleen from FIG. 13E. FIG. 13G, flow cytometry analysis of dendritic cell composition in the spleen from FIG. 13E. The bars in the graph are shown in groups of 4 (from left to right, Uninfected, WT, CARD9−/−, CARD9 R101C) for each different cell type (i.e., CD8a+, CD11b+). FIG. 13H, flow cytometry analysis of neutrophil composition in the spleen from FIG. 13E. FIG. 13I, flow cytometry analysis of inflammatory monocyte composition in the spleen from FIG. 13E. FIG. 13J, flow cytometry analysis of eosinophil composition in the spleen from FIG. 13E. Each experiment was repeated at least 2 times. Error bars represent mean+/−SEM. * p<0.05 from paired t-test.

FIGS. 14A-14D provide a schematic and graphs showing that CARD9 R101C mutation impairs spore clearance in a mouse model of dermatophytosis. FIG. 14A, gating strategy for the skin samples analysis in FIGS. 5A-5L and FIG. 14C. FIG. 14B, CFU from foot pad of mice injected with 10\hat{ }5 live T. rubrum and analyzed at day 9 post injection (n=4 for each genotype). FIG. 14C, flow cytometry analysis of neutrophils composition in the foot pad from FIG. 14B. FIG. 14D, CXCL1 concentration in the foot pad from FIG. 14B measured by ELISA. Each experiment was repeated at least 2 times. Error Bars represent mean+/−SEM. * p<0.05, ** p<0.01 from paired t-test.

FIGS. 15A-15D provide graphs and heat maps showing top differentially expressed genes for immune clusters identified in scRNA-seq analysis. FIG. 15A, top 10 DEGs for each immune cluster. For each cluster, DEGs were defined as genes significantly differentially expressed (FDR P<0.05) for the indicated cluster versus all other immune clusters. FIG. 15B, log-normalized expression of representative markers differentiating the Mono1 and Mono2 clusters. FIG. 15C, log-normalized expression of key markers defining Langerhans cells. FIG. 15D, relative expression of Card9 across all immune clusters. Scale bar indicates z-score.

FIGS. 16A-16N provide heat maps and graphs showing top differentially expressed genes between WT and R101C cells for major immune clusters. FIGS. 16A-16E, heatmaps show top 20 DEGs for each cluster between WT and R101C cells at D2. Row annotations show differential expression of the corresponding gene for the D9 WT vs R101C comparison, WT D2 vs D9 comparison, and R101C D2 vs D9 comparison in the indicated cluster. FIGS. 16F-16H, expression of Ly6C genes among CD45 positive cells in WT or CARD9 R101C skin. FIGS. 16I-16N, expression of cytokines and chemokines among CD45 positive cells in WT or CARD9 skin.

FIGS. 17A-17D provide heat maps and a graph showing top differentially expressed genes for stromal cells and keratinocytes, and additional markers expressed on keratinocytes. FIG. 17A, top 10 DEGs for each stromal cluster. For each cluster, DEGs were defined as genes significantly differentially expressed (FDR P<0.05) for the indicated cluster versus all other stromal clusters. FIG. 17B, top 10 DEGs for each keratinocyte cluster. For each cluster, DEGs were defined as genes significantly differentially expressed (FDR P<0.05) for the indicated cluster versus all other keratinocyte clusters. FIG. 17C, gene set enrichment analysis (GSEA) showing that the keratinocyte clusters identified in the present study are enriched in keratinocyte states. Specifically, for each of our clusters, we used the full list of DEGs for the cluster as defined in FIG. 17B as input to a GSEA pre-ranked analysis using gene signatures for keratinocyte clusters defined in Joost, S. et al. Cell Syst 3, 221-237.e9 (2016), indicated by the text labels beneath the orange bars. High normalized enrichment score (NES) indicates positive enrichment of our clusters in clusters from Joost; low normalized enrichment score (NES) indicates negative enrichment of our clusters in clusters from Joost. FIG. 17D, selected top DEGs for each of the four keratinocyte clusters. Size of bubble indicates mean expression across all cells in each cluster across timepoints and CARD9 genotypes.

FIGS. 18A-18I provide heat maps and graphs showing additional gene families differentially expressed in keratinocytes. FIG. 18A, heatmap showing MitoCarta oxidative phosphorylation/stress genes differentially expressed in keratinocytes at D2. FIG. 18B, heatmap showing MitoCarta oxidative phosphorylation/stress genes differentially expressed in keratinocytes at D9. FIG. 18C, application of the MitoCarta oxidative phosphorylation signature to D2 keratinocyte cells. P-values calculated using two-sided Wilcoxon test. FIG. 18D, Application of the MitoCarta oxidative phosphorylation signature to D9 keratinocyte cells. P-values calculated using two-sided Wilcoxon test. FIG. 18E, heatmap showing keratin family genes differentially expressed between WT and R101C keratinocytes. FIG. 18F, heatmap showing collagen family genes differentially expressed between WT and R101C keratinocytes. FIG. 18G, heatmap showing transcription factors differentially expressed between WT and R101C keratinocytes. FIG. 18H, wound healing genes from gene ontology differentially expressed in each keratinocyte cluster in WT and R101C cells at D2 or D9. Dotted lines indicate fold-change cutoff FIG. 18I, inflammatory response genes from gene ontology differentially expressed in each keratinocyte cluster in WT and R101C cells at D2 or D9. Dotted lines indicate fold-change cutoff

### DETAILED DESCRIPTION OF THE INVENTION

The disclosure features antibodies and antigen binding fragments thereof that specifically bind a phosphorylated S104 amino acid residue in a CARD9 polypeptide, and methods of using such antibodies and antigen binding fragments thereof for the characterization of biological samples, including samples derived from subjects having a propensity to develop severe fungal infection.

The invention is based, at least in part, on the discovery of antibodies that bind a caspase recruitment domain-containing protein 9 (CARD9) comprising a phosphorylated S104 residue. Population genetics continues to identify genetic variants associated with diseases of the immune system and offers a unique opportunity to discover mechanisms of immune regulation. Multiple genetic variants linked to severe fungal infections and autoimmunity have been associated with caspase recruitment domain-containing protein 9 (CARD9). The present disclosure leverages the CARD9 R101C missense variant to uncover a biochemical mechanism of CARD9 activation essential for antifungal responses. It was discovered that CARD9 R101C disrupted a critical signaling switch whereby phosphorylation of S104 releases CARD9 from an autoinhibited state to promote inflammatory responses in myeloid cells. Furthermore, it was shown that CARD9 R101C exerts dynamic effects on the skin cellular contexture during fungal infection, corrupting inflammatory signaling and cell-cell communication circuits. CARD9 R101C mice fail to control dermatophyte infection in the skin, resulting in high fungal burden, yet they show minimal signs of inflammation. Taken together, it was shown how translational genetics can reveal molecular and cellular mechanisms of innate immune regulation.

Accordingly, the disclosure provides antibodies that are directed against and specifically bind to a phosphorylated, a non-phosphorylated S104 amino acid residue in a CARD9 polypeptide, or a phosphorylated S104 residue and a R101C residue present in a CARD9 polypeptide. Without intending to be limiting, such antibodies may be used in methods of selecting a subject or individual who is at risk of, or susceptible to, fungal infections, impaired immune responses to fungal infections, and severe forms of fungal infections. Fungal infections can cause serious disease in a subject, for example, dermatophytosis, which is associated with T. rubrum infection. In embodiments, infection may be caused by Candida species, such as Candida albicans or Candida spp. or by T. rubrum. Such antibodies may also be used in methods of selecting a subject or individual having, or having a propensity to develop, diseases, pathologies, or conditions caused or associated with abnormal CARD9 activation (e.g., increased activation of CARD9 in the subject as compared to a healthy subject), such as, but not limited to, Crohn's disease, inflammatory bowel disease, ankylosing sponditis, and/or IgA nephropathy.

The VH and VL sequences of exemplary anti-phospho-specific CARD9 S104 and other CARD9 antibodies are provided below:

TABLE 1
Anti-phospho-specific CARD9 S104
Antibody Sequences

| Ab# | Chain | Sequence | CDR1 | CDR2 | CDR3 |
|---|---|---|---|---|---|
| 1 | VH | MGWSWIFLFL | GYSF | INPY | CAIW |
| | | LSGTAGVHSE | TGQT | NGGT | DYVL |
| | | VQLQQSGPEL | (SEQ | (SEQ | DYW |
| | | VKPGASMKMS | ID | ID | (SEQ |
| | | CKASGYSFTG | NO: | NO: | ID |
| | | QTMHWVKQTH | 6) | 7) | NO: |
| | | GKNLEWIGLI | | | 8) |
| | | NPYNGGTKYN | | | |
| | | QIFTGKATLT | | | |
| | | VDKSSSSAYM | | | |
| | | ELLSLTSEDS | | | |
| | | AVYFCAIWDY | | | |
| | | VLDYWGQGTS | | | |
| | | VTVSSAKTTP | | | |
| | | PSVYPLAPGS | | | |
| | | AAQTNSMVTL | | | |
| | | GCLVK | | | |
| | | (SEQ ID | | | |
| | | NO: 5) | | | |
| 1 | VL | MKLPVRLLVL | QSLL | KVS | CSQS |
| | | MFWIPASSSD | HSNG | | THIP |
| | | VVMTQTPLSL | KTY | | PTF |
| | | PVSLGDQASI | (SEQ | | (SEQ |
| | | SCRSSQSLLH | ID | | ID |
| | | SNGKTYLHWY | NO: | | NO: |
| | | LQKPGQSPKL | 10) | | 11) |
| | | LIYKVSNRFS | | | |
| | | GVPDRFSGSG | | | |
| | | SGTDFTLKIS | | | |
| | | RVEAEDLGVY | | | |
| | | FCSQSTHIPP | | | |
| | | TFGGGTKLEI | | | |
| | | KRADAAPTVS | | | |
| | | IFPPSSEQLT | | | |
| | | SGGASVVCF | | | |
| | | (SEQ ID | | | |
| | | NO: 9) | | | |

### Caspase Recruitment Domain-Containing Protein 9 (CARD9) Polypeptide (Protein)

Caspase recruitment domain-containing protein 9 (CARD9) is an adaptor polypeptide (protein) that signals downstream of immunoreceptor tyrosine-based activation motif (ITAM)-containing receptors in myeloid cells. Recognition of fungal cell wall carbohydrates by C-type lectin receptors (CLRs) and subsequent downstream signaling through CARD9 is critical for activation of nuclear factor-κB (NFκB) and initiation of antifungal immune response (Drummond et al., 2018). The CLR Dectin-1 engages fungal carbohydrates, and through the cascade of events leads to CARD9 activation, promoting the formation of a complex consisting of CARD9, Bcl10 and Malt1 (CBM) (Strasser et al., 2012). Post-translational modifications (PTMs) such as phosphorylation and ubiquitination play an important role in regulating CARD9 activity and its ability to form the CBM complex. Despite significant progress, key questions remain regarding how CARD9 is activated and how understanding the structural dynamics of this process can be exploited for drug discovery. In this regard, human genetics affords the opportunity to uncover completely unexpected mechanisms of inflammatory signal transduction.

The mechanistic regulation of CARD9 signaling was assessed as described herein by taking a translational genetics approach. The natural emergence of coding variants in CARD9 associated with fungal disease provide valuable genetic leads for structure-function studies aimed at deciphering how CARD9 activity is controlled. Without intending to be limiting, the studies described herein focused on CARD9 R101C, which is linked to dermatophytosis in humans, to demonstrate that R101 regulates a phosphorylation event that is essential for CARD9 activity and protection from fungal infection in mouse models. Thus, it was demonstrated how genetics can be applied to uncover biochemical mechanisms of CARD9 signaling and contextualize the consequences of disease-associated variants in protective tissue immunity in vivo.

### The Role of CARD9 in Anti-Fungal Immune Responses and Immune Defense Mechanisms

Insights from monogenic immunodeficiencies offer an unique opportunity to dissect mechanisms of protein function, signal transduction, and regulation of immune responses. Genetic variants associated with these phenotypes are rare, due to evolutionary selection, and typically result in loss of expression. However in some cases, immunodeficiencies are associated with variants that do not affect protein expression but alter its function instead. Uncovering the mechanistic basis for how these variants alter immune function can offer new strategies for therapeutic interventions that modulate immune function with greater precision. This general approach has proven to be successful in oncology, where detailed mechanistic study of somatic mutations in oncogenes such as BRAF or KRAS have led to insights that fueled the development of targeted cancer therapeutics. Here, mechanisms of antifungal immunity through translational genetics are uncovered, with the rationale that understanding germline encoded missense variants associated with immunodeficiency can reveal mechanisms of immunity that may have value for future development of anti-inflammatory therapeutics to treat autoimmunity.

Fungi are a natural part of the human commensal flora. However, invasive fungal infections are a growing concern worldwide as they are one of the major causes of infectious disease related mortality. Because the available treatments for such fungal infections are limited, new and effective treatment products and methods are of critical importance for patients having, at risk or having, or susceptible to such infections, especially severe infections. In addition, methods of identifying and selecting subjects and individuals who are at risk of, or susceptible to, having an impaired immune response to an infection by fungi and/or acquiring serious or severe forms of disease associated with fungal infection, are provided by detecting in a subject or individual a variant form of CARD9, i.e., CARD9 R101C variant, and/or a lack or absence of phosphorylated amino acid residue S104 in the CARD9 protein in a sample obtained from the subject or individual.

Most cases of disease or pathology are driven by opportunistic fungi, which can lead to life-threatening infections, especially in immunocompromised patients, and cause fungal sepsis and damage to vital organs such as kidneys, brain, liver, lungs or heart. The appearance of more drug-resistant strains, such as Candida auris, is problematic in hospital and residential care facilities and represents a major health burden. With an increase in immunocompromised patients and resistant strains, consideration must be given to understanding disease mechanisms and developing new therapeutics. Genetic variants associated with fungal disease offer an opportunity to learn about protein functions and modes of immune regulation.

Given the increase in the number of patients immunocompromised due to chronic illnesses, medication, or invasive medical procedures, as well as the emergence of drug resistant fungal species, there is a need to develop effective methods for detecting and selecting those subjects and individuals who are at risk of having or are susceptible to a serious or severe fungal infection that can be life-threatening. Such subjects and individuals can then be treated appropriately, e.g., with more potent anti-fungal agents and drugs, or combinations of anti-fungal agents and drugs, to ameliorate or alleviate the severity of infection and to provide therapy and treatment for co-emerging fungal infections.

Studies of patients without any known immunodeficiencies who suffer from fungal infections have led to the discovery of defects in specific immune genes and signaling pathways that can result in uncontrolled fungal invasion. In addition, genome-wide association studies (GWAS) have identified several common genetic variants that increase susceptibility to candidemia without full penetrance (Kumar et al., 2014). In particular, CARD9 has been implicated in antifungal defense. At least 24 known loss-of-function mutations that lead to increased susceptibility to fungal infections have been found

GWAS studies have also implicated CARD9 variants in the pathogenesis of diseases, such as inflammatory bowel disease, Crohn's disease, ankylosing spondylitis, primary sclerosing cholangitis, and IgA nephropathy. Such GWAS studies highlight the importance of understanding how genetic variation in CARD9 is involved in fine-tuning the immune response, thus providing targets for new therapeutic agents, e.g., small molecule drugs and compounds and biologics, e.g., polypeptides, peptides, such as antibodies and treatment methods.

### Methods

Methods and uses related to the discovery of the described CARD9 R101C variant polypeptide, the phosphorylated S104 amino acid residue in the CARD polypeptide and its association with the normal activity and functioning of a CARD protein containing R101, and phospho-specific antibodies generated against the phosphorylated S104 residue in the CARD9 protein are described.

In an aspect, a method of treating a subject who is at risk of, or susceptible to, serious or severe disease, or an impaired immune response associated with a fungal infection is provided, in which the method involves administering to the subject a potent anti-fungal treatment or treatment regimen, wherein the subject has been selected as being at risk of, or susceptible to, serious or severe disease, or an impaired immune response, associated with a fungal infection by detecting in the subject's sample (i) a CARD9 variant polypeptide comprising the mutation R101C compared with a wildtype CARD9 polypeptide; and (ii) a CARD9 polypeptide in which amino acid residue S104 is not phosphorylated. The determination of a CARD9 variant polypeptide comprising the mutation R101C compared with a wildtype CARD9 polypeptide can be carried out using methods well known and practiced by a skilled practitioner in the art, for example, without limitation, antibody detection assays using the anti-phospho-specific CARD9 S104 antibodies described herein. In particular embodiments, the anti-phospho-specific CARD9 S104 antibodies described herein are used in an immunoassay. Immunoassay typically utilizes an antibody to detect the presence or level of a biomarker (e.g., phosphorylated CARD9 S104) in a sample.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

It is within the purview of a medical practitioner and/or the subject or individual who is selected as needing a potent, or stronger-than-normal anti-fungal treatment based on the practice of the method to be administered the appropriate therapeutic regimen or treatment, or combination of treatments and/or treatment regimens. By way of nonlimiting example, antifungal medicines and drugs suitable for treatment and therapeutic use include echinocandin; flucytosine; voriconazole; caspofungin; micafungin; posaconazole; isavuconazole; clotrimazole (Canesten), econazole, miconazole, terbinafine (Lamisil), fluconazole (Diflucan), ketoconazole (Daktarin), itraconazole, nystatin (Nystan), amphotericin (e.g., amphotericin B), and/or griseofulvin. Without intending to be limiting, antifungal medicines and drugs may be administered to a subject or individual (patient) in the form of a cream, ointment, gel, spray, capsule, tablet, liquid, injection, suppository, or pessary.

In another aspect, a method of selecting a subject who is at risk of, or susceptible to, serious or severe disease, or an impaired antifungal immune response, associated with a fungal infection, in which the method involves contacting a sample obtained from the subject with an antibody, or an antigen-binding portion thereof, that specifically binds to a phosphorylated S104 amino acid residue in a CARD9 polypeptide; detecting binding of the antibody, or an antigen-binding portion thereof, to the phosphorylated S104 amino acid residue in the CARD9 polypeptide; and selecting the subject as being at risk of, or susceptible to, serious or severe disease, or an impaired immune response associated with the fungal infection.

In embodiments of the above methods, the subject's sample can be, without limitation, a cell, blood, serum, plasma, or other body fluid or tissue sample. In the methods, the disease may be one or more of immunodeficiency disease, autoimmunity, inflammatory disease, inflammatory bowel disease (IBD), Crohn's disease, ankylosing spondylitis, primary sclerosing cholangitis, IgA nephropathy, or dermatophytosis. In embodiments of the methods, the fungal infection is caused by, without limitation, Candida albicans, Candida auris, other Candida species, Trichophytom rubrum (T. rubrum), and other infectious fungal species.

Methods of treating a disease (e.g., a fungal infection, particularly a fungal infection in a subject having a CARD9 R101C mutation), or symptoms thereof, are provided. The methods comprise treating a selected subject having a fungal infection, where the subject is selected by contacting a biological sample obtained from the subject with an antibody described herein and selecting the subject when a reduction in the amount of phosphorylated CARD9 S104 is detected, relative to a reference. In some embodiments, the biological sample is a cell sample. In some embodiments, the biological sample is a sample obtained from a wound. In some embodiments, the biological sample is a sample obtained from a site of fungal infection.

The methods include administering an effective amount of an anti-fungal agent, to a subject (e.g., a mammal), in particular, a human subject. In some embodiments, the subject is a selected subject, where the subject is selected by contacting a biological sample obtained from the subject with the anti-phospho-specific CARD9 S104 antibodies described herein and selecting the subject if a reduction in the amount of phosphorylated CARD9 S104 is detected, relative to a reference. In some embodiments, the anti-fungal agent is an anti-fungal agent suitable for treating a fungal infection in an immunocompromised subject. Exemplary anti-fungal agents suitable for treating fungal infections in immunocompromised subjects may be found in Low CY, Rotstein C. (2011) Emerging fungal infections in immunocompromised patients. F1000 Med Rep. 3:14. In some embodiments, the anti-fungal agent is one or more of: echinocandin; flucytosine; voriconazole; caspofungin; micafungin; posaconazole; isavuconazole; clotrimazole (Canesten); econazole; miconazole; terbinafine (Lamisil); fluconazole (Diflucan); ketoconazole (Daktarin); itraconazole; nystatin (Nystan); amphotericin (e.g., amphotericin B); and/or griseofulvin.

In some embodiments, the methods herein include administering to the subject (including a human subject identified as in need of such treatment) an effective amount of an anti-fungal agent, effective to treat a fungal infection. The treatment methods are suitably administered to subjects, particularly humans, having a CARD9 mutation (e.g., an R101C mutation or a mutation preventing phosphorylation of S104), which renders the subject vulnerable to fungal infection. The fungal infection vulnerability is as compared to a subject lacking the CARD9 mutation or having a wild type CARD9 gene, and may be characterized by one or more of: reduced survival rates when infected with a fungal infection; reduced fungal spore clearance; higher fungal burden when infected with a fungal infection; reduced activation of immune signaling pathways (e.g., IL1 and Cxcl2/Cxcr2 pathways); impairment to immune response to fungal infection (e.g., reduced monocyte or neutrophil infiltration at site of fungal infection or reduced monocyte or neutrophil inflammatory response to fungal infection); and/or disruption of wound healing caused by fungal infection (e.g., characterized by alteration or modulation of keratinocyte behavior, such as keratinocyte proliferation).

The disease may be caused by a fungal infection. Exemplary fungal infections include infections by Candida spp., Aspergillus spp., Fusarium spp., Scedosporium spp., Trichophytom rubrum (T. rubrum), and Zygomycosis.

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., assay by the anti-phospho-specific CARD9 S104 antibodies described herein), marker analysis, family history, and the like, including an opinion of the subject or a health care provider. A subject undergoing treatment can be a non-human mammal, such as a veterinary subject, or a human subject (also referred to as a “patient”).

In addition, prophylactic methods of preventing or protecting against a disease (e.g., fungal infections, particularly fungal infection in a subject having a CARD9 mutation), or symptoms thereof, are provided. Such methods comprise administering an anti-fungal agent to a selected subject, where the subject is selected by contacting a biological sample obtained from the subject with the anti-phospho-specific CARD9 S104 antibodies described herein and selecting the subject if a reduction in the amount of phosphorylated CARD9 S104 is detected, relative to a reference.

The anti-fungal agent can be administered to a subject by any of the routes normally used for introducing an anti-fungal agent into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

### Generation and Screening of Phospho-Specific CARD9 S104 Antibodies

Phospho-specific antibodies (e.g., monoclonal antibodies) that specifically bind to phosphorylated S104 in a CARD9 protein are provided and described herein. In some embodiments, the phospho-specific antibodies described herein that specifically bind to phosphorylated S104 in a CARD9 protein (i.e., anti-phospho-specific CARD9 S104 antibodies) include a sequence or a complementarity determining region (CDR) sequence provided in Table 1. Methods for generating antibodies against a protein of interest are known in the art.

When animals are immunized with antigens they respond by generating a polyclonal antibody response comprised of many individual monoclonal antibody specificities. It is the sum of these individual specificities that make polyclonal antibodies useful in so many different assays. Individual monoclonal antibodies were originally isolated by immortalizing individual B cells using hybridoma technology (Kohler and Milstein, Nature 256, 495, 2011), in which B cells from an immunized animal are fused with a myeloma cell. With the advent of molecular biology, in vitro methods to generate antibodies, including the antibodies as described herein, against proteins of interest have been developed.

The terms “antigen of interest” or “target protein” are used herein interchangeably and refer generally to the agent recognized and specifically bound by an antibody.

An antibody is a polypeptide chain-containing molecular structure with a specific shape that specifically binds an epitope, where one or more non-covalent binding interactions stabilize the complex between the molecular structure and the epitope. In one embodiment, an antibody molecule is an immunoglobulin (e.g., IgG, IgM, IgA, IgE, IgD). Antibodies from a variety of sources, e.g. human, rodent, rabbit, cow, sheep, pig, dog, or fowl are considered “antibodies.” Numerous antibody coding sequences have been described; and others may be raised by methods well-known in the art.

For example, antibodies, including anti-phospho-specific CARD9 S104 antibodies antibodies, or antigen binding fragments thereof may be produced by genetic engineering. Antibody coding sequences of interest include those encoded by native sequences, as well as nucleic acids that, by virtue of the degeneracy of the genetic code, are not identical in sequence to a wild-type nucleic acid sequence. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues). Variants also include fragments of the polypeptides disclosed herein, particularly biologically active fragments and/or fragments corresponding to functional domains. Techniques for in vitro mutagenesis of cloned genes are known. Also included in some aspects and embodiments herein are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated fully herein by reference).

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although apparently complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in some aspects and embodiments herein may be produced by synthesizing a variable light chain region and a variable heavy chain region. Combinations of antibodies are also of interest, e.g. diabodies, which comprise two distinct Fv specificities.

Immunoglobulins may be modified post-translationally, e.g. to add chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of some aspects and embodiments herein.

Screening of Libraries for Antibodies that Bind to Phosphorylated S104 in CARD9 Protein

Methods for high throughput screening of libraries of antibodies or antigen-binding fragments thereof for polypeptides capable of phospho-specific S104 binding include, without limitation, display techniques including phage display, bacterial display, yeast display, mammalian display, ribosome display, mRNA display, and cDNA display. The use of phage display to isolate ligands that bind biologically relevant molecules has been reviewed, e.g., in Felici et al. (Biotechnol. Annual Rev. 1:149-183, 1995), Katz (Annual Rev. Biophys. Biomol. Struct. 26:27-45, 1997), and Hoogenboom et al. (Immunotechnology 4:1-20, 1998). Several randomized combinatorial peptide libraries have been constructed to select for polypeptides that bind different targets, e.g., cell surface receptors or DNA (reviewed by Kay (Perspect. Drug Discovery Des. 2, 251-268, 1995), Kay et al., (Mol. Divers. 1:139-140, 1996)). Proteins and multimeric proteins have been successfully phage-displayed as functional molecules (see EP 0349578A, EP 4527839A, EP 0589877A; Chiswell and McCafferty (Trends Biotechnol. 10, 80-84 1992)). In addition, functional antibody fragments (e.g. Fab, single-chain Fv [scFv]) have been expressed (McCafferty et al. (Nature 348: 552-554, 1990), Barbas et al. (Proc. Natl. Acad Sci. USA 88:7978-7982, 1991), Clackson et al. (Nature 352:624-628, 1991)). These publications are hereby incorporated by reference in their entirety.

In addition to generating anti-phospho-specific CARD9 S104 antibodies and antigen-binding fragments thereof of some aspects and embodiments herein, in vitro display techniques (e.g., those described herein and those known in the art) also provide methods for improving the affinity of an anti-phospho-specific CARD9 S104 antibodies of some aspects and embodiments herein. For instance, rather than screening libraries of antibodies and fragments thereof containing completely randomized hypervariable regions, one can screen narrower libraries of antibodies and antigen-binding fragments thereof that feature targeted mutations at specific sites within hypervariable regions. This can be accomplished, e.g., by assembling libraries of polynucleotides encoding antibodies or antigen-binding fragments thereof that encode random mutations only at particular sites within hypervariable regions. These polynucleotides can then be expressed in, e.g., filamentous phage, bacterial cells, yeast cells, mammalian cells, or in vitro using, e.g., ribosome display, mRNA display, or cDNA display techniques in order to screen for antibodies or antigen-binding fragments thereof that specifically bind a phosphorylated S104 epitope(s) with improved binding affinity. Yeast display, for instance, is well-suited for affinity maturation, and has been used previously to improve the affinity of a single-chain antibody to a KD of 48 fM (Boder et al., Proc Natl Acad Sci USA 97:10701, 2000).

Additional in vitro techniques that can be used for the generation and affinity maturation of anti-phospho-specific CARD9 S104 polypeptides (e.g., single-chain polypeptides, antibodies, and antigen-binding fragments thereof) of some aspects and embodiments herein include the screening of combinatorial libraries of antibodies or antigen-binding fragments thereof for functional molecules capable of specifically binding a phosphorylated CARD9 S104. Combinatorial antibody libraries can be obtained, e.g., by expression of polynucleotides encoding randomized hypervariable regions of an antibody or antigen-binding fragment thereof in a eukaryotic or prokaryotic cell. This can be achieved, e.g., using gene expression techniques described herein or known in the art. Heterogeneous mixtures of antibodies can be purified, e.g., by Protein A or Protein G selection, sizing column chromatography), centrifugation, differential solubility, and/or by any other standard technique for the purification of proteins. Libraries of combinatorial libraries thus obtained can be screened, e.g., by incubating a heterogeneous mixture of these antibodies with a relevant peptide derived from CARD9 that has been immobilized to a surface for a period of time sufficient to allow antibody-antigen binding. Non-binding antibodies or fragments thereof can be removed by washing the surface with an appropriate buffer (e.g., a solution buffered at physiological pH (approximately 7.4) and containing physiological salt concentrations and ionic strength, and optionally containing a detergent, such as TWEEN-20). Antibodies that remain bound can subsequently be detected, e.g., using an ELISA-based detection protocol (see, e.g., U.S. Pat. No. 4,661,445; incorporated herein by reference).

Additional techniques for screening combinatorial libraries of polypeptides (e.g., antibodies, and antigen-binding fragments thereof) for those that specifically bind CARD9-derived peptides include the screening of one-bead-one-compound libraries of antibody fragments. Antibody fragments can be chemically synthesized on a solid bead (e.g., using established split-and-pool solid phase peptide synthesis protocols) composed of a hydrophilic, water-swellable material such that each bead displays a single antibody fragment. Heterogeneous bead mixtures can then be incubated with a CARD9-derived peptide that is optionally labeled with a detectable moiety (e.g., a fluorescent dye) or that is conjugated to an epitope tag (e.g., biotin, avidin, FLAG tag, HA tag) that can later be detected by treatment with a complementary tag (e.g., avidin, biotin, anti-FLAG antibody, anti-HA antibody, respectively). Beads containing antibody fragments that specifically bind a CARD9-derived peptide can be identified by analyzing the fluorescent properties of the beads following incubation with a fluorescently-labeled antigen or complementary tag (e.g., by confocal fluorescent microscopy or by fluorescence-activated bead sorting; see, e.g., Muller et al. (J. Biol. Chem., 16500-16505, 1996); incorporated herein by reference). Beads containing antibody fragments that specifically bind CARD9-derived peptides can thus be separated from those that do not contain high-affinity antibody fragments. The sequence of an antibody fragment that specifically binds a CARD9-derived peptide can be determined by techniques known in the art, including, e.g., Edman degradation, tandem mass spectrometry, matrix-assisted laser-desorption time-of-flight mass spectrometry (MALDI-TOF MS), nuclear magnetic resonance (NMR), and 2D gel electrophoresis, among others (see, e.g., WO 2004/062553; incorporated herein by reference).

### Methods of Identifying Antibodies and Ligands

Methods for high throughput screening of antibody, antibody fragment, and ligand libraries for molecules capable of binding a phosphorylated CARD9 S104 can be used to identify anti-phospho-specific CARD9 S104 antibodies as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind phospho-S104 of CARD9.

Host Cells for Expression of Antibodies that Bind Phosphorylated S104 of CARD9

Mammalian cells can be co-transfected with polynucleotides encoding the antibodies of some aspects and embodiments herein, which are expressed as recombinant polypeptides in the host cell. In one embodiment, a mammalian cell is co-transfected with polynucleotides encoding the heavy and light chains of the antibody that are expressed as an antibody protein in the cell.

It is possible to express antibodies (e.g., the anti-phospho-specific CARD9 S104 antibodies described herein, or antigen-binding fragments thereof) in either prokaryotic or eukaryotic host cells. In certain embodiments, expression of polypeptides (e.g., anti-phospho-specific CARD9 S104 antibodies, or antigen-binding fragments thereof) is performed in eukaryotic cells, e.g., mammalian host cells, for optimal secretion of a properly folded and immunologically active antibody. Exemplary mammalian host cells for expressing the recombinant antibodies or antigen-binding fragments thereof of some aspects and embodiments herein include Chinese Hamster Ovary (CHO cells) (including DHFR CHO cells, described in Urlaub and Chasin (1980, Proc. Nat. Acad. Sci. USA 77:4216-4220), used with a DHFR selectable marker, e.g., as described in Kaufman and Sharp (1982, Mol. Biol. 159:601-621), NSO myeloma cells, COS cells, HEK293T cells, SP2/0, NIH3T3, and BaF3 cells. Additional cell types that may be useful for the expression of antibodies and fragments thereof include bacterial cells, such as BL-21(DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Polypeptides (e.g., anti-phospho-specific CARD9 S104 antibodies, or antigen-binding fragments thereof) can be recovered from the culture medium using standard protein purification methods. Host cells can also be used to produce portions of intact antibodies, such as Fab fragments or scFv molecules. Also included in some aspects and embodiments herein are methods in which the above procedure is varied according to established protocols known in the art. For example, it can be desirable to transfect a host cell with DNA encoding either the light chain or the heavy chain (but not both) of an anti-phospho-S104 CARD9 antibody of some aspects and embodiments herein in order to produce an antigen-binding fragment of the antibody.

Once an anti-phospho-specific CARD9 S104 antibody or an antigen-binding fragment thereof of some aspects and embodiments herein has been produced by recombinant expression, it can be purified by any method known in the art, such as a method useful for purification of an immunoglobulin molecule, for example, by chromatography (e.g., ion exchange, affinity, particularly by affinity for phosphorylated CARD9 S104 after Protein A or Protein G selection, and sizing column chromatography), centrifugation, differential solubility, or by any other standard technique for the purification of proteins. Further, the anti-phospho-specific CARD9 S104 antibodies of some aspects and embodiments described herein, or antigen-binding fragments thereof, can be fused to heterologous polypeptide sequences described herein or otherwise known in the art to facilitate purification.

Once isolated, an anti-phospho-specific CARD9 S104 antibody, or antigen-binding fragments thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a Superdex™ 75 column (Pharmacia Biotech AB, Uppsala, Sweden).

### High Throughput Assays/Screening Assays

High throughput screening (HTS) assays involve the use of automated equipment to rapidly test thousands to millions of candidate or test samples for biological activity at the cellular, molecular, pathway, or model organism level. In its most common form, -ITS is an experimental process in which 10³-10⁶ small molecule compounds of known structure are screened in parallel. Other substances, such as chemical mixtures, natural product extracts, oligonucleotides, proteins, peptides, and antibodies, may also be screened. Because HTS typically aims to screen 100,000 or more samples per day, relatively simple and automation-compatible assay designs, robotic-assisted sample handling, and automated data processing are critical. HTS is commonly used to identify compounds (called hits or leads) having pharmacological or biological activity. These are used as starting points for medicinal chemical optimization during pharmacological probe or drug discovery and development. Typically, HTS assays are performed in microtiter plates, e.g., in 96-, 384-, or 1536-well formats, while traditional HTS can test each compound in a compound library at a single concentration, such as 10 μM.

Other solid phase assay formats can also be used, such as microfluidic chips for cell-based screening systems (Young, 2013). Another HTS approach involves fragment-based screening (Schulz and Hubbard, 2009) in which a smaller library of small molecules (small molecule chemical compounds are evaluated for binding to a target. Fragments that bind to the target are then assembled into larger compounds which are then evaluated for binding to the target.

Quantitative high throughput screening (qHTS) is a method of testing compounds at multiple concentrations using an HTS platform. Concentration response curves are generated for each compound tested immediately after the screen is performed. Recently, qHTS has become popular in toxicology analyses because it more fully characterizes the biological effects of chemicals and decreases the rates of false positives and false negatives.

The primary goal of ITS is to identify through compound library screenings candidates that affect the target in the desired way, so-called “hits” or “leads.” This is usually achieved by employing liquid handling devices, robotics, plate readers as detectors, and dedicated software for instrumentation control and data processing.

In general, HTS involves four steps:

### Preparation of Samples and Compound Libraries:

Samples are usually of cellular or biochemical nature, depending on the assay to be run. High-throughput screening necessitates that samples are prepared in an arrayed format. The key platform or sample carrier used is therefore the microplate. Typical formats include 384-, 1536-, or 3456-well plates. The nature of the sample and of the detection assay may affect the choice of the microplate format and its color. Screening facilities usually keep their compound library collections stored in so-called “stock plates”. Stock plates are not directly used in experiments. Instead, when needed, compounds from a stock plate are “copied” to an assay plate through a pipetting station.

### Establishment of a Method Suitable for Automation

Automation is an important element in HTS as the conversion of a benchtop to an automated high-throughput screening assay enforces specific constraints affecting the practical assay design. Ideally, a HITS assay is performed in a single well, with a low amount of reagents (miniaturization), and minimal or no further manipulation than injection of the sample/compound to be tested. Accordingly, the choice of the optimal detection mode and assay has to be subordinated to automation issues. For more information on the identification of false positives in screening attempts please see AN 359: Identification of false positives in a HTRF® screen for small molecule inhibitors of PD-1/PD-L1.

Establishing a stringent assay and an effective quality control method are major issues when setting up an automated screen. The clearer the distinction between negative and positive controls, the higher the possibility to obtain high-quality data with a neglectable number of false-negative but especially false-positive results.

### Configuration of a Robotic Workstation

The scope of a robotic platform is to autonomously manage multiple plates simultaneously, significantly speeding up data acquisition. Robotic platforms for high-throughput screenings range from simple automated liquid handling machines to multidimensional workstations performing multiple functions. This is usually achieved with the support of one or more mechanical arms. Typically, a robotic system manages microplates from station to station for several steps such as reagent addition, mixing, incubation, and detection.

### Acquisition and Handling of Data

In high-throughput screenings data acquisition is usually performed by an optical measurement, quantifying the amount of light “produced” by the sample. Different readouts such as fluorescent or luminescent detection, colorimetry, or light scatter (turbidity) are available. Common detection modes include fluorescence intensity and polarization, fluorescence resonance energy transfer (FRET), which provides assays to directly detect the oligomerization state and oligomerization degree of membrane proteins in their native environment; time-resolved fluorescence (e.g.: HTRF®, LANCE®, etc), luminescence (e.g., NanoBRET), and AlphaScreen®. As understood by the skilled practitioner in the art, FRET is a distance-dependent physical process by which energy is transferred nonradiatively from an excited molecular fluorophore (the donor) to another fluorophore (the acceptor) by means of intermolecular long-range dipole-dipole coupling.

Depending on the biological question to be answered, data quality as well as cost-effectiveness, different light-based detection readouts may be chosen. Specialized instrumentation, like multi-mode microplate readers, can sequentially perform different experiments or apply different detection protocols on the wells. The output thereof is a grid of numeric values.

### FRET Assays

Fluorescence is the light signal we can detect when a fluorophore absorbs energy at a specific wavelength and is excited to emit light at a higher wavelength as it returns to its ground state.

During FRET a donor fluorophore becomes excited by a light source and transfers its energy to a nearby acceptor fluorophore. The acceptor fluorophore absorbs the energy to produce a detectable light emission signal. This process results in the loss of fluorescence of the donor and gain of fluorescence of the acceptor, both of which can be measured.

For FRET to occur several conditions must be met:

### Proximity.

The donor and acceptor fluorophores must be close to one another for the FRET process to be efficient. FRET efficiency (E) is defined by the equation E=Ro6/(Ro6+r6), where Ro is the Förster radius and r is the actual distance between the two fluorophores. The Förster radius is the distance at which 50% of the excitation energy is transferred from the donor to the acceptor, and the Ro value usually lies between 10-100 Å (1-10 nm). FRET pairs with an Ro value towards the higher end of this range are often preferred due to the increased likelihood of FRET occurrence.

### Spectral Overlap.

The emission spectrum of the donor fluorophore must overlap the absorption spectrum of the acceptor fluorophore. The greater the degree of spectral overlap, the more likely FRET is to occur.

### Dipole Orientation.

Energy transfer from the donor fluorophore to the acceptor fluorophore occurs via intermolecular dipole-dipole coupling. The spatial relationship between the donor emission dipole moment and the acceptor absorbance dipole moment is described by the orientation factor, κ2, which has values ranging from 0 (all dipoles are perpendicular) to 4 (all dipoles are parallel). Dipole orientation between the two fluorophores is typically assumed to be random due to rapid molecular rotation and is taken to equal the statistical average (a value of ⅔) for most calculations of Ro.

### Applications of FRET Technology

FRET is an extremely powerful method of identifying potential molecular interactions and can be used in techniques such as flow cytometry, immunocytochemistry, immunohistochemistry and ELISA. FRET is also ideally suited to High Throughput Screening (HTS) since it is simple, sensitive and easily automated.

One popular use of FRET is to identify an interaction between two biomolecules, for example, the binding of a ligand to a receptor; a FRET signal can only be detected when the biomolecules are in close proximity by virtue of a binding event.

FRET relies on the use of high quality labeled reagents. Depending on the intended assay setup these could be antibodies, proteins or peptides.

### Optimal Fluorochrome Pairs for FRET

Nonlimiting FRET pairs have been developed for producing proteins (antibodies) conjugated or linked to detectable compounds for use in FRET assays, such as Streptavidin-Phycoerythrin (RPE)-Allophycocyanin (APC); RPE-Cy5, RPE-Cy5.5, RPE-Cy7, RPE-DyLight®650, RPE-APC/Cy5.5, Fluorescein-RPE, and APC-DyLight750. A wide range of fluorescent-labeled antibodies, proteins and dyes, covering the spectrum from UV to far-infrared, are commercially available for use in FRET assays, e.g., Abcam (Cambridge, UK), ThermoFisher Scientific (Waltham, MA).

### Kits

The disclosure provides kits comprising antibodies for characterizing the activation and/or phosphorylation state of a CARD9 polypeptide in a biological sample (e.g., bone marrow, bone marrow monocyte, bone marrow dendritic cell) of a subject. In some embodiments, the kit comprises a sterile container which contains a composition comprising antibodies described herein; such containers can be boxes, ampoules, bottles, vials, tubes, bags, pouches, blister-packs, or other suitable container forms known in the art. Such containers can be made of plastic, glass, laminated paper, metal foil, or other materials suitable for holding antibodies and fragments thereof.

If desired an antibody described herein is provided together with instructions for using the antibody for characterizing the susceptibility of a subject to a fungal infection. The instructions will generally include information about the use of the composition in characterizing a biological sample. In other embodiments, the instructions include at least one of the following: description of the antibody; precautions; warnings; clinical studies; and/or references. The instructions may be printed directly on the container (when present), or as a label applied to the container, or as a separate sheet, pamphlet, card, or folder supplied in or with the container.

The practice of aspects and embodiments herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of some aspects and embodiments herein, and, as such, may be considered in making and practicing some of the aspects and embodiments herein. Particularly useful techniques for particular embodiments will be discussed in the sections that follow.

The following examples are put forth so as to provide those of ordinary skill in the art with a complete disclosure and description of how to make and use the assay, screening, and therapeutic methods of some aspects and embodiments herein, and are not intended to limit the scope of the various aspects and embodiments herein.

### EXAMPLES

### Example 1: CARD9 Linker Residues R101 and S104 are Required to Elicit Cytokine Responses

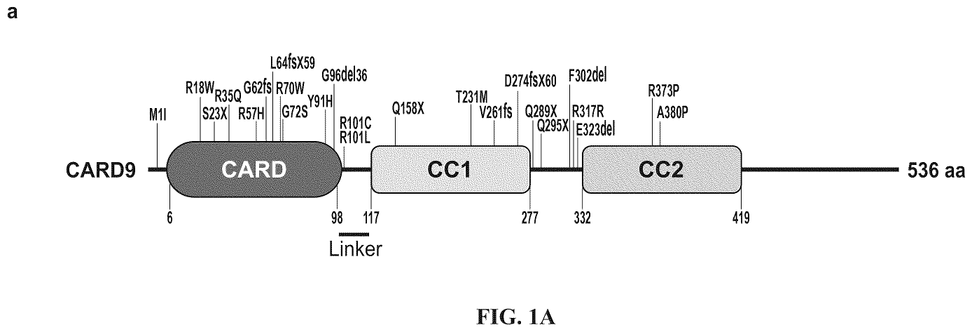

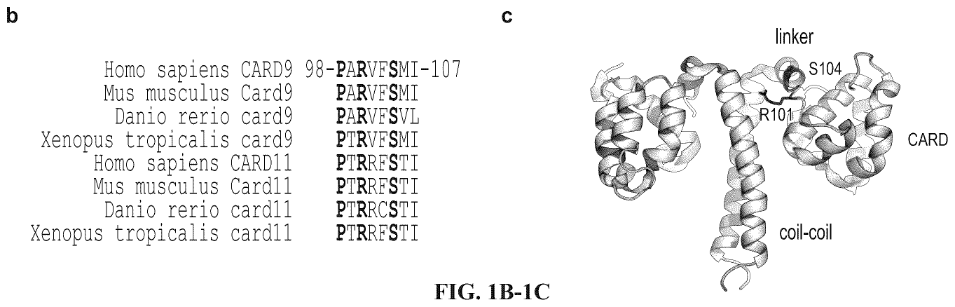

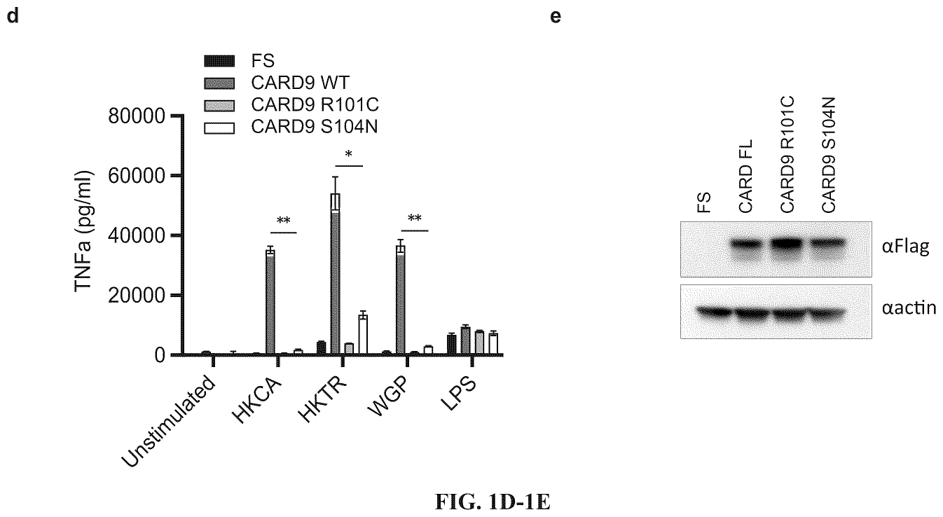

CARD9 adopts an autoinhibited conformation, and it remains unclear how structural dynamics relieve autoinhibition to promote filamentation and subsequent assembly of the CARD11-BCL10-MALT1 (CBM) signalosome (CBM) complex (Holliday et al., 2019). The autoinhibited conformation of CARD9 is stabilized by interactions between the coil-coil (CC1) domain and key amino acid residues in the CARD domain and the linker region between CARD and CC1 domains (Holliday et al., 2019). Without intending to be bound by theory, this interface may be a key region that functions as an activating switch in response to post-translational modifications. To gain further insight into the mechanisms regulating CARD9 activity, genetic variants were evaluated that predispose a subject having such variants to severe fungal disease. Only two CARD9 variants, R101C and R101L, are located in the linker region, which is a notably conserved region among both CARD9 and CARD11 in higher vertebrates (FIGS. 1A-1B, Vaezi et al., 2018, Grumach et al., 2015, Lantemier et al., 2013). Both variants are in proximity to site S104, which comprises a common phosphorylation motif RXXS that is disrupted when arginine is replaced with another amino acid at position 101 (FIG. 1C). To determine whether the RXXS motif is essential for CARD9 function and signaling downstream of Dectin-1 receptor in response to fungal stimuli, full-length CARD9 (CARD FL) or CARD9 mutants were ectopically expressed either in CARD9 knock-out (CARD9^{-/-}) bone marrow-derived dendritic cells (BMDCs). It was observed that CARD9 R101C, the risk variant for fungal infection, completely ablated the cytokine response to heat killed C. albicans (HKCA), heat killed dermatophyte T. rubrum (HKTR) or whole glucan particles (WGP), but not to LPS (FIGS. 1D-IE and FIG. 8A). Similarly, S104 is required for CARD9-dependent signaling, as substitution of S104N is incapable of phosphorylation and ablates cytokine production downstream of Dectin-1 ligands (FIGS. 1D-IE and FIG. 8A). The same results were obtained using bone marrow-derived macrophages (BMDMs) (FIGS. 8B-8C).

### Example 2: CARD9 Activation Through S104 Phosphorylation is Impaired by the R101C Variant Implicated in Fungal Disease

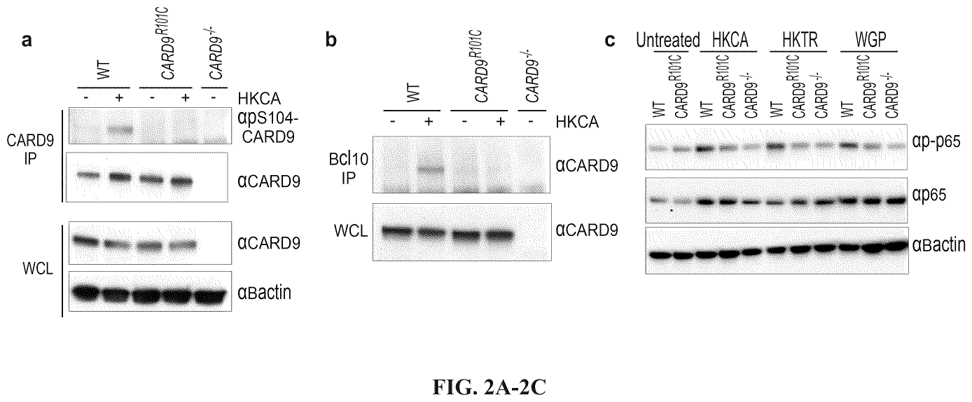

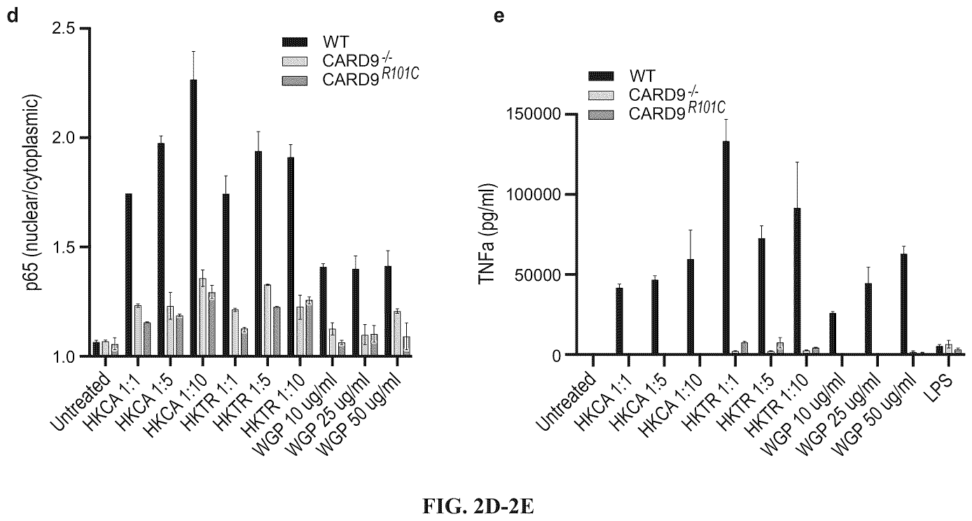

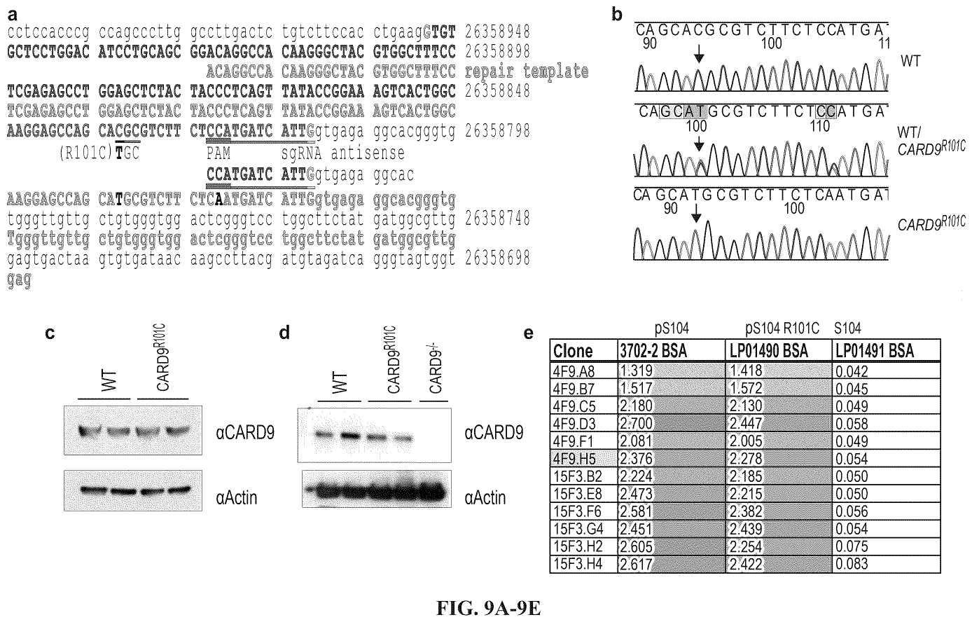

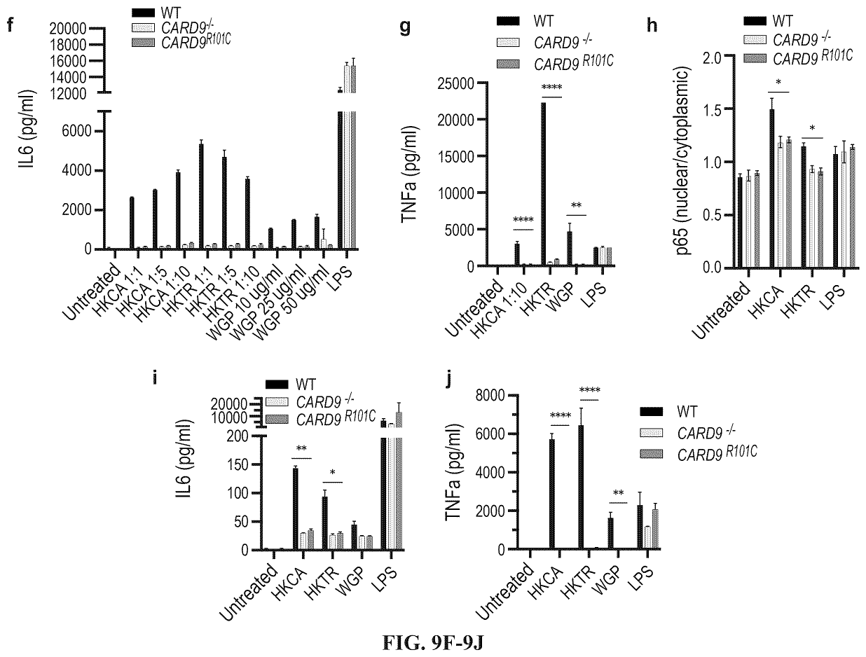

To directly assess the relationship between a CARD9 R101C risk variant and S104 phosphorylation, (i) a phosphospecific antibody for pS104 and (ii) a mouse model of CARD9 R101C were generated. In the CARD9 R101C mouse, analysis of BMDCs and splenic CD11c cells confirmed unimpaired expression of CARD9 R101C protein relative to WT (FIGS. 9A-9D). To study the phosphorylation event on CARD9 S104 a mouse monoclonal phospho-specific CARD9 S104 antibody was generated that recognizes S104 phosphorylation independently of R101. Antibody specificity was verified by ELISA against different peptides, and clones were intentionally selected that recognize pS104 in the context of either R101 or C101 to determine if R101C substitution can impair phosphorylation of S104 (FIG. 9E). The phospho-S104 specific antibody was then used to determine the phosphorylation state in primary cells upon stimulation with fungi by immunoprecipitating CARD9 from either WT or CARD9 R101C BMDCs. Following HKCA stimulation, S104 phosphorylation in WT cells, but not in CARD9 R101C BMDCs (FIG. 9A) was observed. To determine the status of CBM complex following stimulation, Bcl10 was immunoprecipitated from either WT or CARD9 R101C BMDCs and verified inducible interaction with CARD9. While stimulation-dependent association of Bcl10 and CARD9 was observed in WT cells, this did not occur in CARD9 R101C cells (FIG. 2B). Consistent with these findings, impaired NFkB activation was also observed by evaluating phosphorylation and nuclear translocation of p65 in R101C BMDCs after stimulation with fungal ligands such as HKCA and HKTR as well as WGP (FIGS. 2C-2D). BMDCs from CARD9 R101C mutant also showed reduced production of cytokines following stimulation (FIG. 2E and FIG. 9F). Both BMDMs and monocytes isolated from CARD9 R101C mutant mice also exhibited impaired responses to fungal stimulation (FIGS. 9G-9J). Without intending to be bound by theory, these results suggest that the R101C variant disrupts a critical kinase recognition motif, impairing S104 phosphorylation and subsequent activation of CARD9 signaling—resulting in lack of immune response to fungal stimulation.

### Example 3: CARD9 R101 Functions as a Signaling Switch Activated by S104 Phosphorylation

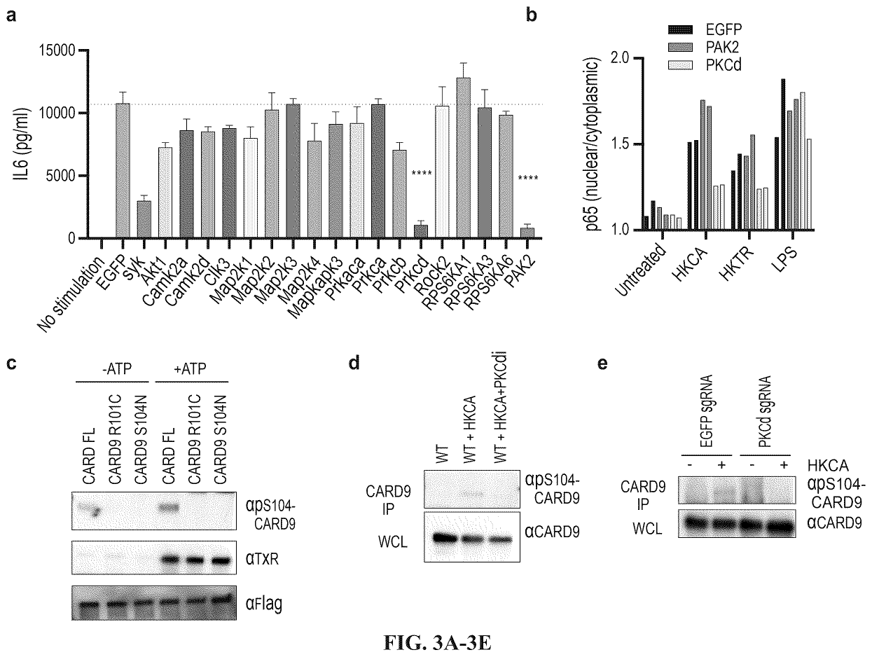

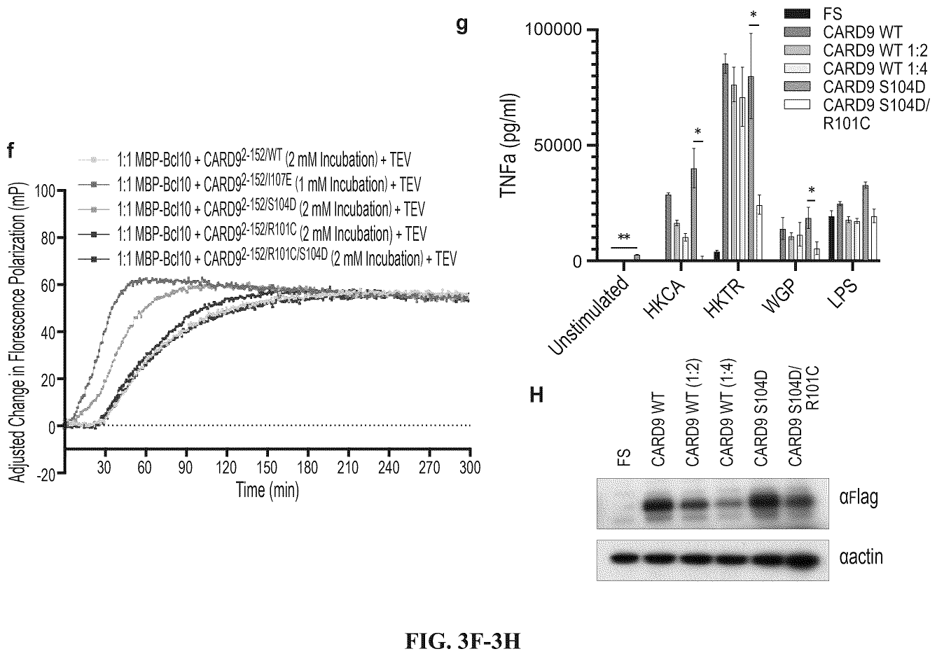

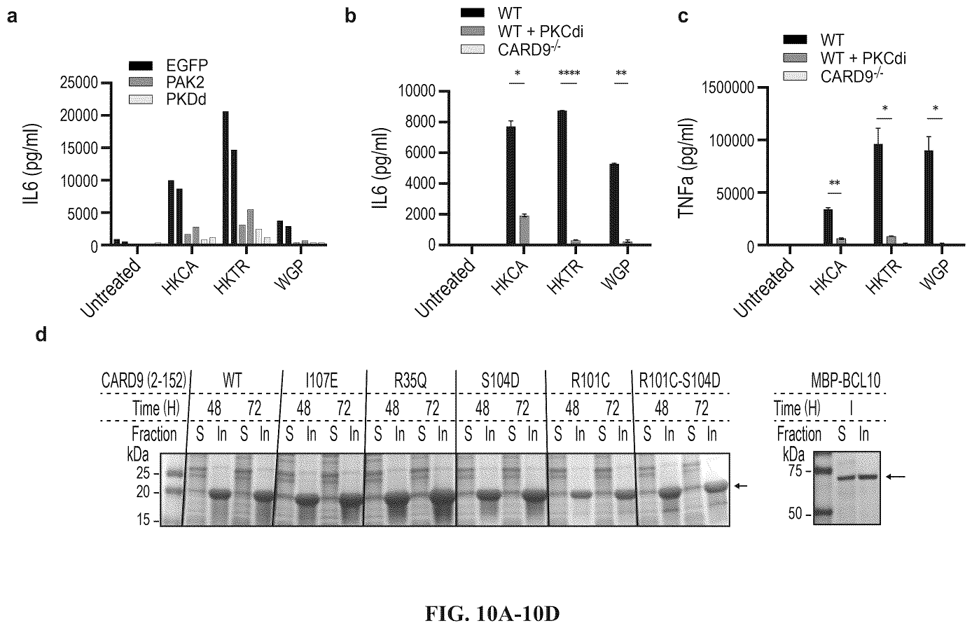

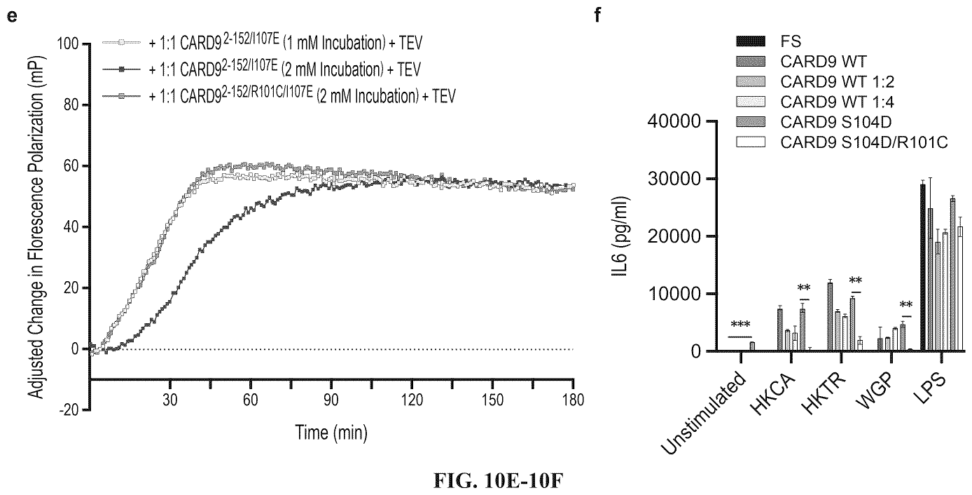

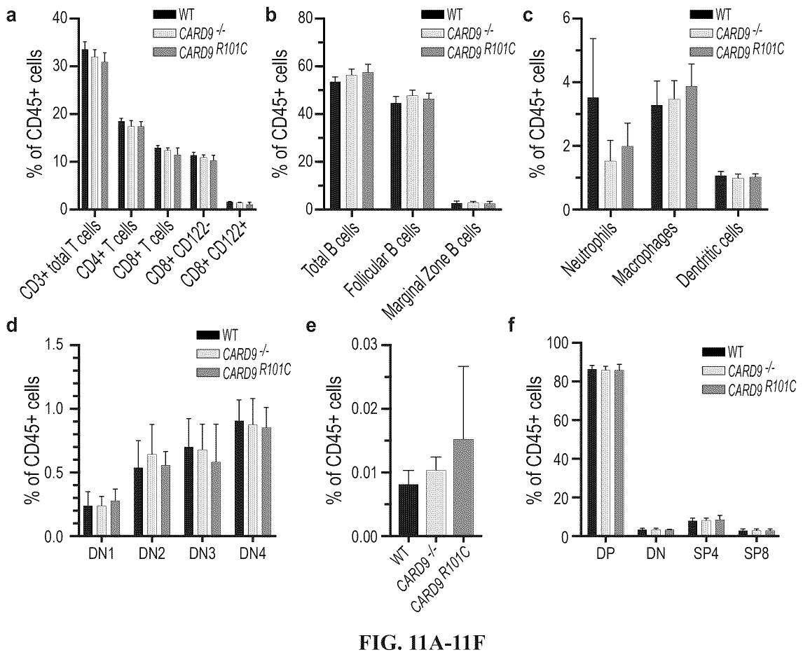

In order to identify the kinase that phosphorylates CARD9 at S104, a CRISPR screen was performed by perturbing key kinases and then measuring cytokine production in response to Dectin-1 stimulation. Kinases were screened that phosphorylate RXXS motifs, (Johnson et al., 2023) are expressed in BMDCs, and/or were identified as CARD9 interacting proteins (Cao et al., 2015, Xu et al., 2020). Given that these kinases were screened for activity in a cytokine assay, it was anticipated that this approach would identify the kinase that phosphorylates CARD9 in addition to any kinases acting upstream, downstream, or parallel to CARD9. For example, KO of the tyrosine kinase Syk was performed as a positive control, because it is known to act upstream of CARD9 and ablate cytokine production in response to Dectin-1 stimulation. Out of 18 tested RXXS kinases, CRISPR KO of PAK2 and PKCd resulted in reduction of IL-6 in response to Dectin-1 stimulation (FIG. 3A). To implicate the kinase that directly phosphorylates CARD9 at S104, secondary screening assays were performed. P65 nuclear translocation was first evaluated, as this process is more proximal to CARD9 activation, occurs on a time scale that correlates with S104 phosphorylation (FIGS. 2A and 2D), and is completely ablated by R101C mutation (FIG. 2D). Only PKCδ KO showed reduction in NFkB signaling following Dectin-1 stimulation, as seen by reduced p65 translocation to nucleus (FIG. 3B and FIG. 10A). Since PKCδ was previously identified to phosphorylate CARD9 at T231 (Strasser et al., 2012), whether it can also phosphorylate S104 was determined. In vitro phosphorylation assay demonstrated that PKCd does indeed phosphorylate S104, and this phosphorylation requires an intact RXXS motif in the linker domain (FIG. 3C). Of note, CARD9 phosphorylation at TXR motifs like T231 remained intact (FIG. 3C). Having shown that PKCδ is sufficient to phosphorylate S104, next was to formally demonstrate its requirement. Accordingly, both pharmacologic inhibition of PKC with 10 uM Sotrastaurin and genetic KO of PKCδ using CRISPR via nucleofection led to inhibition of CARD9 S104 phosphorylation in BMDCs stimulated with HKCA (FIGS. 3D-3E and FIGS. 10B-10C).

Next was to further define how R101 and phosphorylation of S104 by PKCδ regulate its activation. Prior work demonstrated that hydrophobic interactions between the CARD domain, linker region, and CC1 domain maintain CARD9 in an autoinhibited state (Holliday et al., 2019). Disruption of this interaction through mutagenesis (I107E) releases CARD9 from the autoinhibited state and induces NFkB activation as well as formation of Bcl10-templating filaments (Holliday et al., 2019). It was first tested if phosphomimetic mutation S104D in CARD9 is sufficient to catalyze Bcl10 filamentation. Thus, a fluorescence polarization-based Bcl10 polymerization assay (Holliday et al., 2019, Qiao et al., 2013, Holliday et al, 2018), was employed, and demonstrated that WT CARD9 (aa 2-152) in the autoinhibited state had no effect on the rate of Bcl10 filamentation, whereas S104D accelerated the process (FIG. 3F and FIG. 10A). Both variants were compared to CARD9 1107E that has been previously described to accelerate the filament formation (Holliday et al., 2019). Moreover, the double mutant of CARD9 R101C/S104D had no effect on Bcl10 polymerization, indicating that R101C overrides and prevents the activation of CARD9 by S104D (FIG. 3F and FIG. 10D). However, CARD9 R101C/I107E was competent to accelerate filament formation, indicating that R101C does not completely incapacitate the protein, rather it can be activated by I107E-mediated release of the CARD domain (FIG. 10E).

Having demonstrated the requirement for R101 and S104 phosphorylation in CARD9 signaling, it was hypothesized that phosphomimetic mutation at the S104 site might promote or potentiate cytokine responses in a CARD9-dependent manner in cells. Indeed, expression of CARD9 S104D in CARD9^{-/-} BMDCs led to increased baseline cytokine production independent of stimulation (untreated) without impairing Dectin-1 or LPS responses (FIGS. 3G-3H and FIG. 10F). To determine if S104 and R101 function cooperatively in cells, a double mutant S104D/R101C was generated, and demonstrated that S104D was not sufficient to rescue impaired cytokine production of R101C after fungal stimulation (FIGS. 3G-3H and FIG. 10F). Similar results were obtained when CARD9 S104D and S104D/R101C variants were expressed in CARD9−/− BMDMs (FIGS. 8B-8C). Overall, the data is consistent with a model in which phosphorylation at S104 causes a conformational switch allowing for the CARD domain to release from the coiled coil domain and catalyze Bcl10 polymerization for downstream cytokine production. This conformational switch in CARD9 requires R at position 101. Thus, the R101C mutation both disrupts the RXXS phospho-site motif and also disarms the switch mechanism that allows for the release of CARD domain.

### Example 4: CARD9 R101C Mice are Predisposed to Systemic Fungal Infection

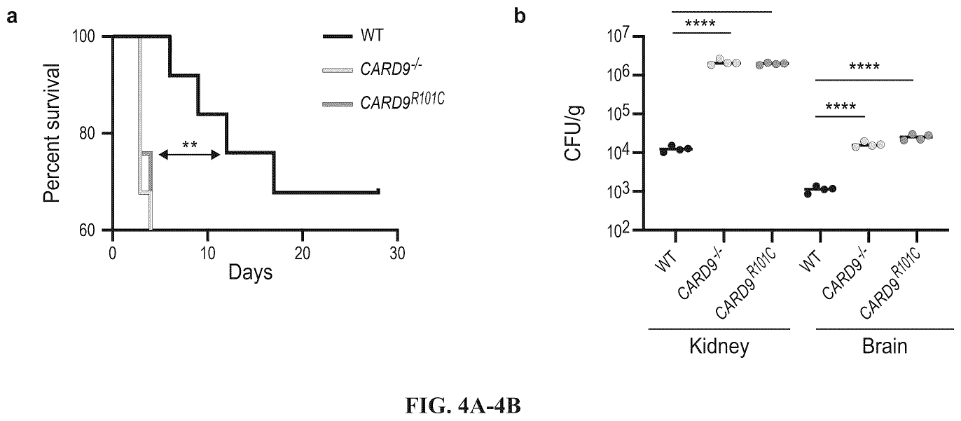

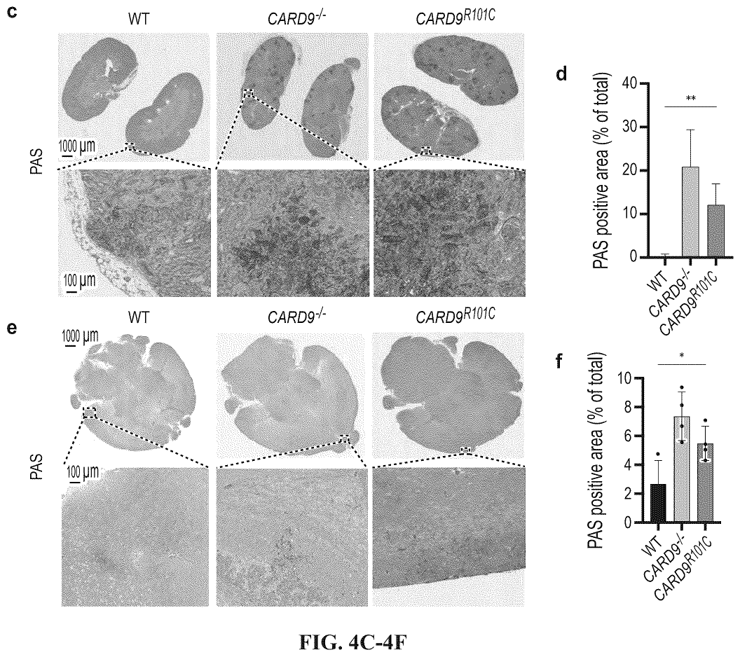

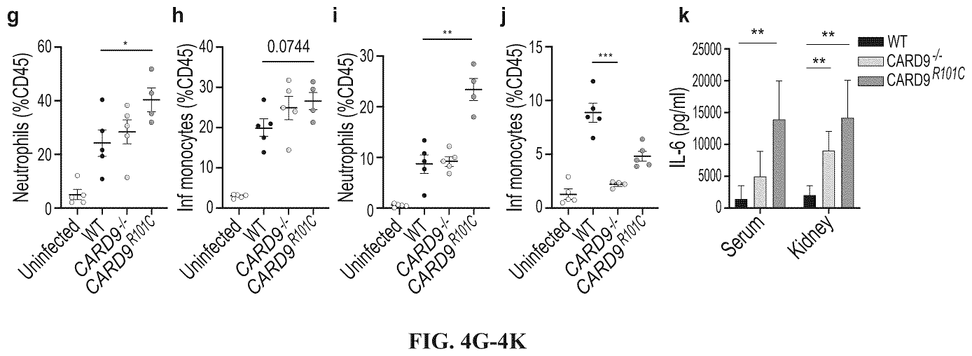

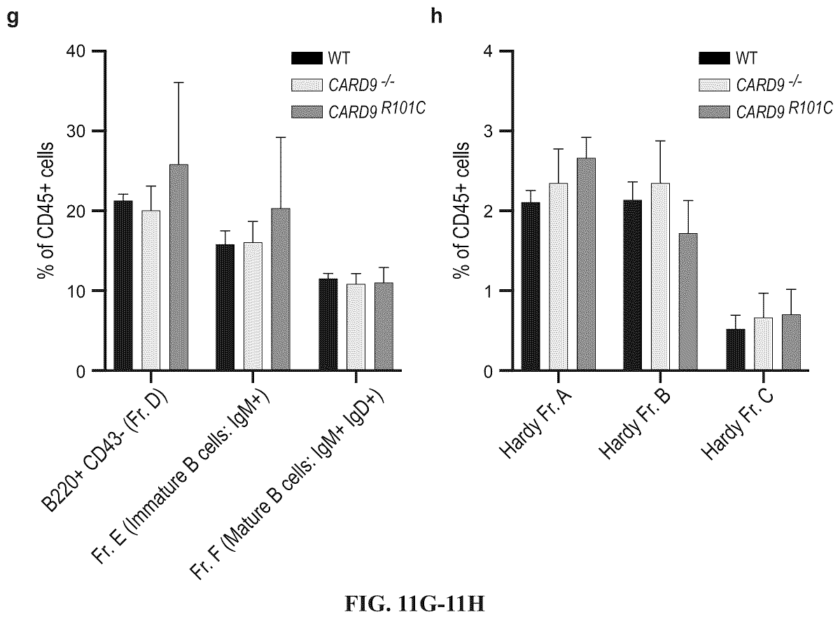

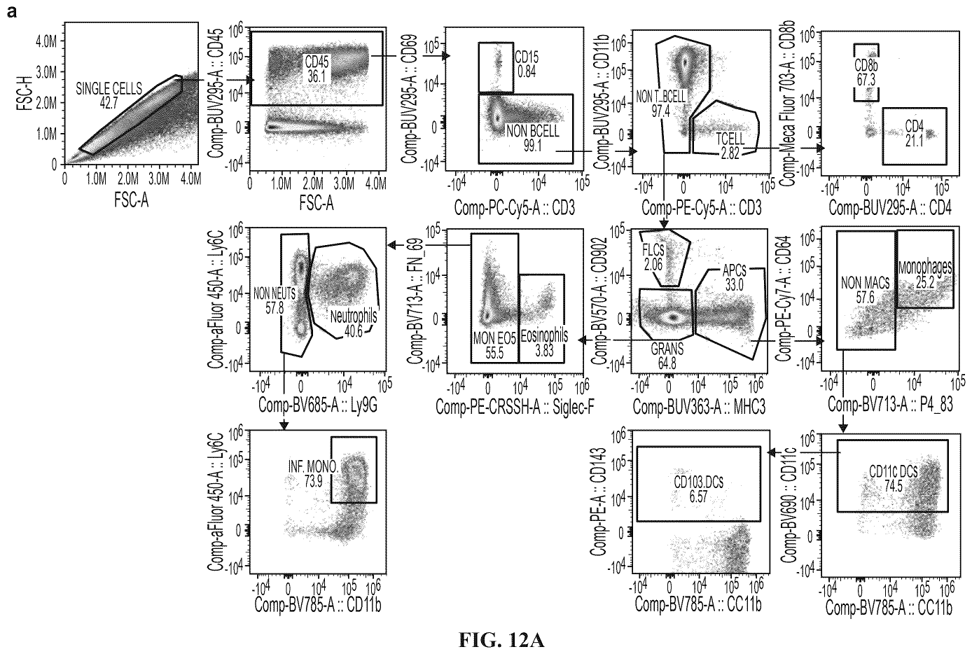

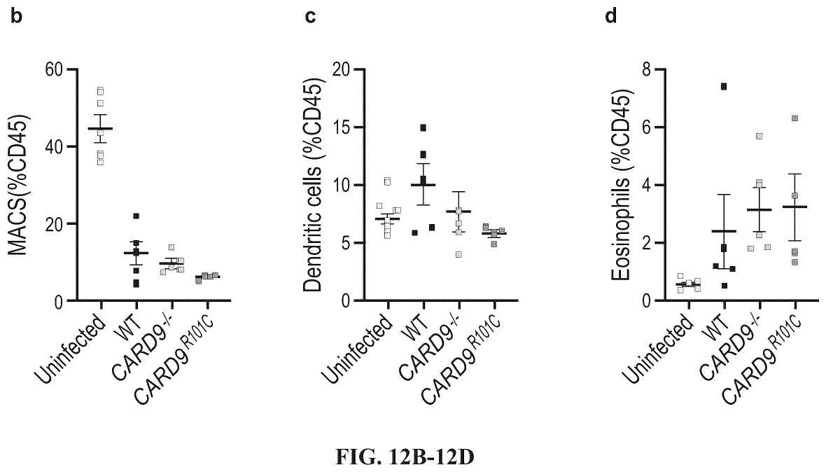

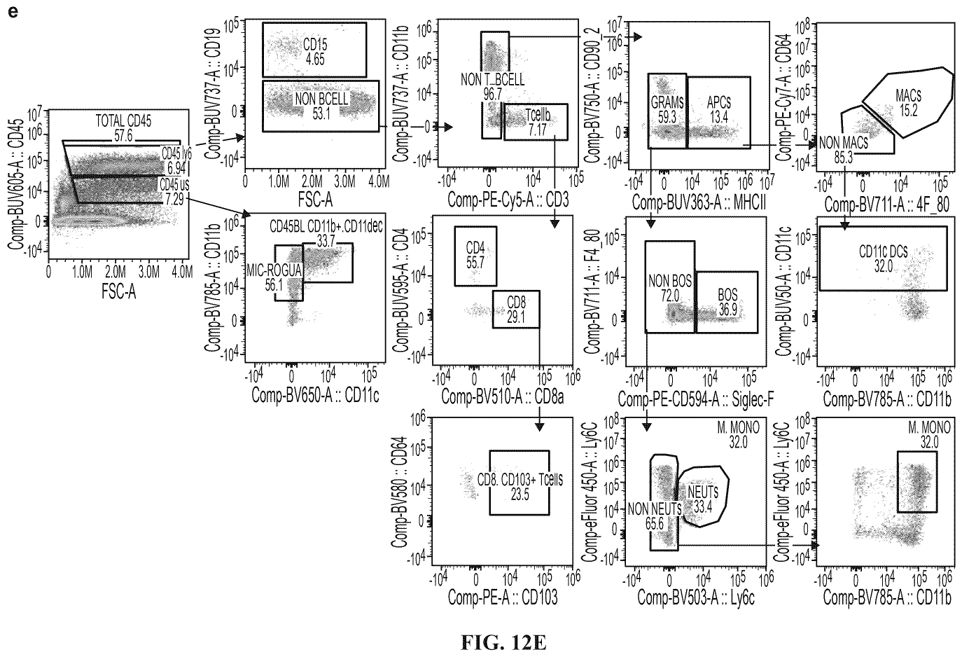

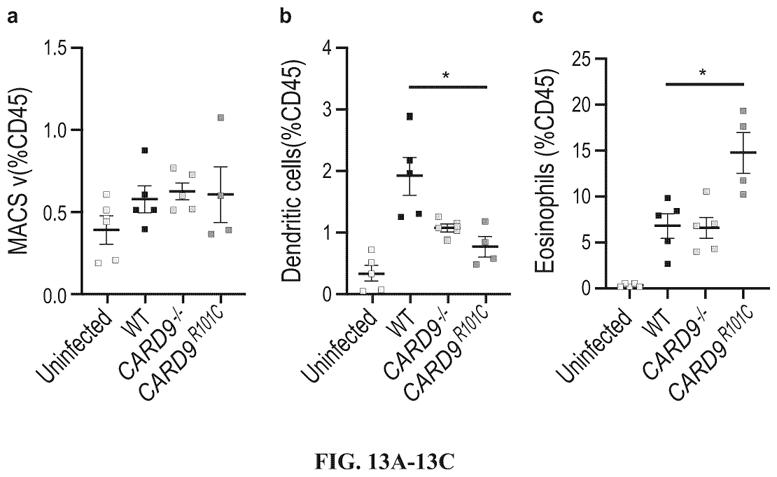

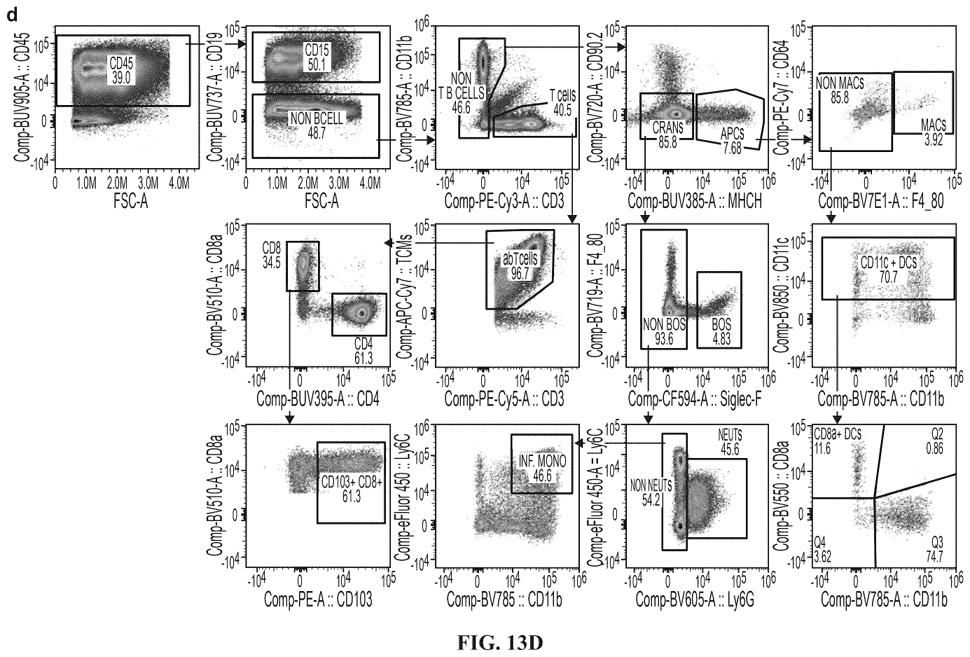

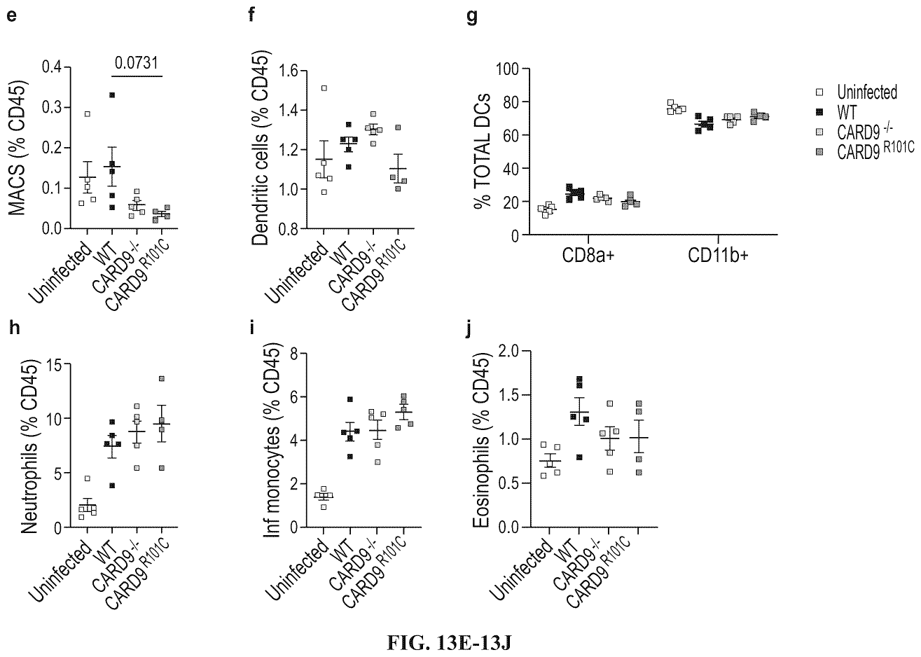

How CARD9 R101C impacts global antifungal responses in vivo was tested. Extensive immunophenotyping in CARD9 R101C mutant mice did not reveal any changes in immune cell subset distribution at the steady state in the spleen, thymus or bone marrow; and spontaneous development of fungal infection was not observed (FIGS. 11A-11H). Next, a model of systemic candidiasis induced by intravenous infection was utilized. Mice harboring the R101C variant, similar to CARD9^{-/-} mice, showed severely reduced survival, with most of the mice succumbing to the Candida albicans infection between days 2-4 post infection (FIG. 4A). Further analysis of the mice at day 2 post-infection revealed that CARD9 R101C mice displayed 100 times higher fungal burden in kidney and 10 times higher fungal burden in the brain, which closely mimics the phenotype observed in CARD9^{-/-} mice (FIG. 4B). Histopathological analysis of both kidneys and brains obtained from infected mice showed significantly increased presence of fungal mats in both organs in the mutant mice (FIGS. 4C-4F). Immunophenotyping of kidney and brain demonstrated increased neutrophil infiltration in both organs from CARD9 R101C mice and severe impairment of inflammatory monocyte infiltration only in the brain of the mutant mice (FIGS. 4G-4J, FIGS. 12A-12E, and FIGS. 13A-13C). Additionally, brains from CARD9 R101C mice showed increases in eosinophil infiltration and decreases in presence of dendritic cells (FIGS. 13B-13C). Spleens of CARD9 R101C mice showed reductions in macrophage infiltration and in CD8a positive dendritic cells (FIGS. 13D-13J). Cytokine analysis of infected mice demonstrated signs of increased systemic inflammation in both CARD9^{-/-} and CARD9 R101C mice as seen by IL-6 both in serum and in kidney (FIG. 4K). Taken together these data point to the importance of R101 in regulating the activity of CARD9 and innate immunity in the context of systemic antifungal responses.

### Example 5: CARD9 R101C Mutation Impairs Spore Clearance in a Mouse Model of Dermatophytosis

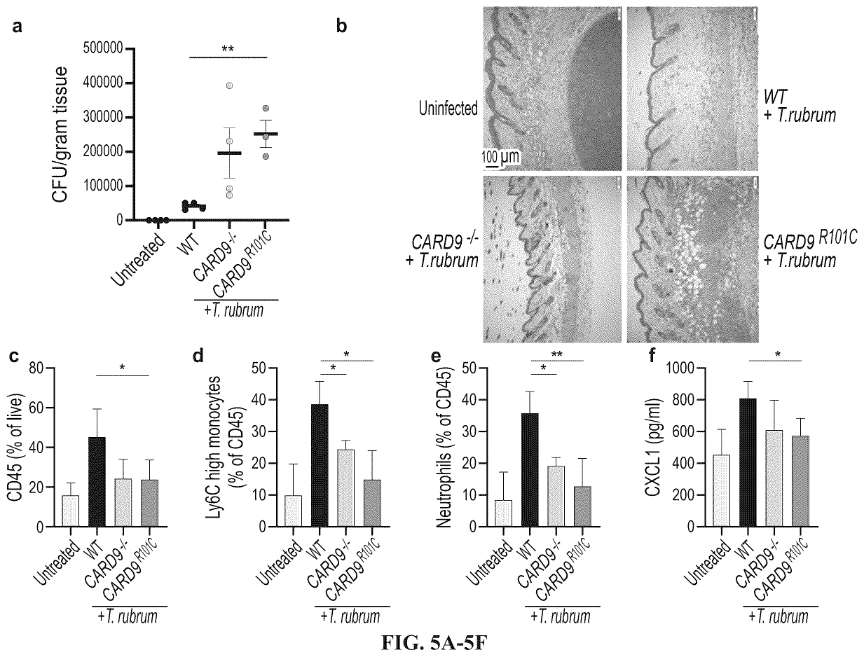

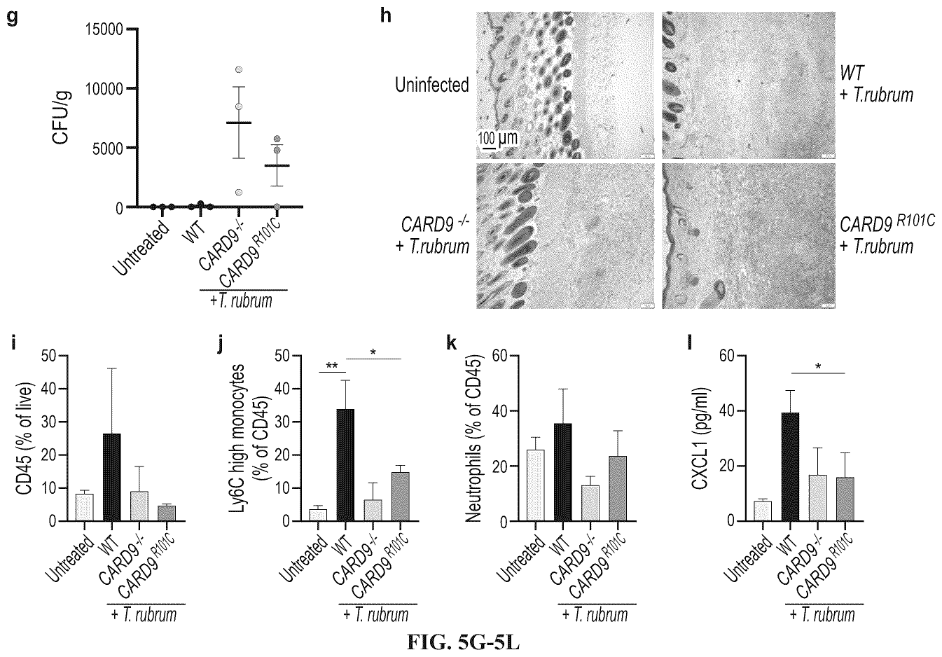

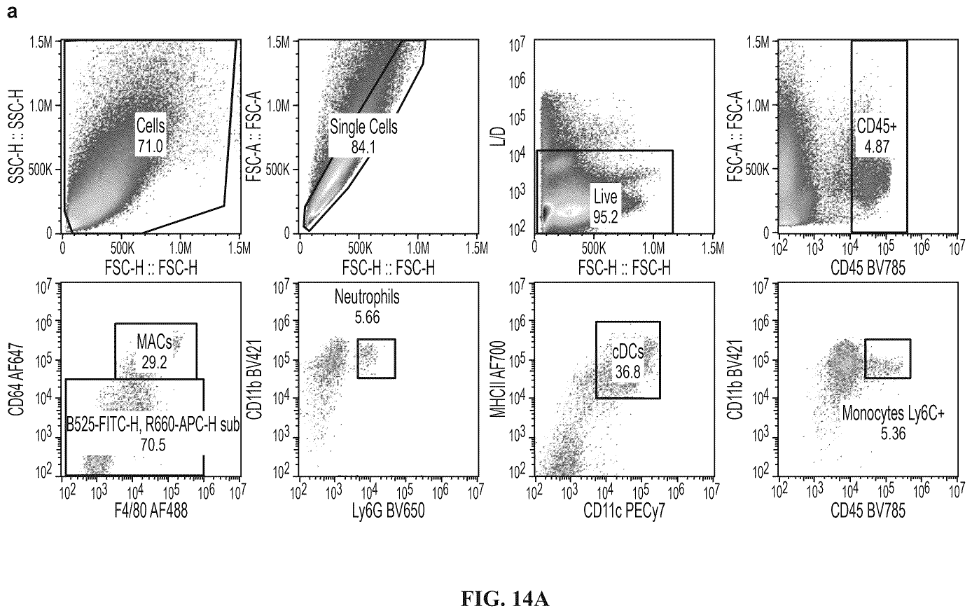

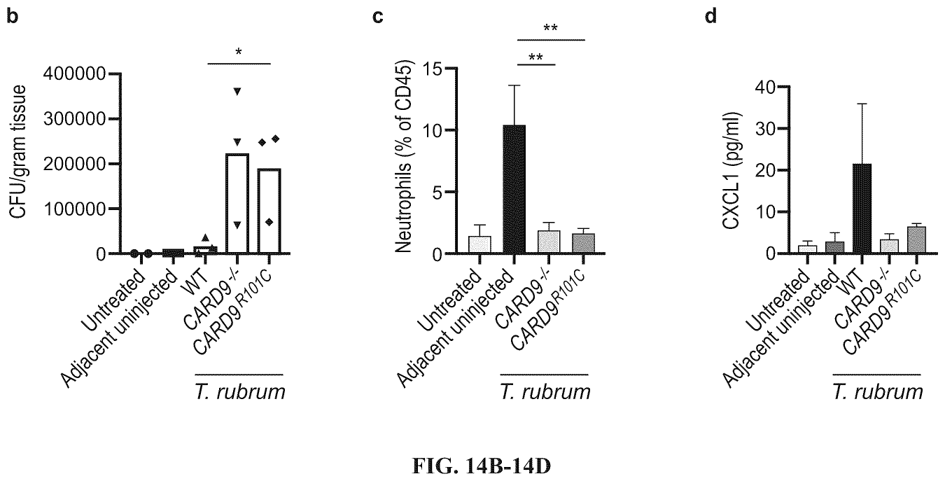

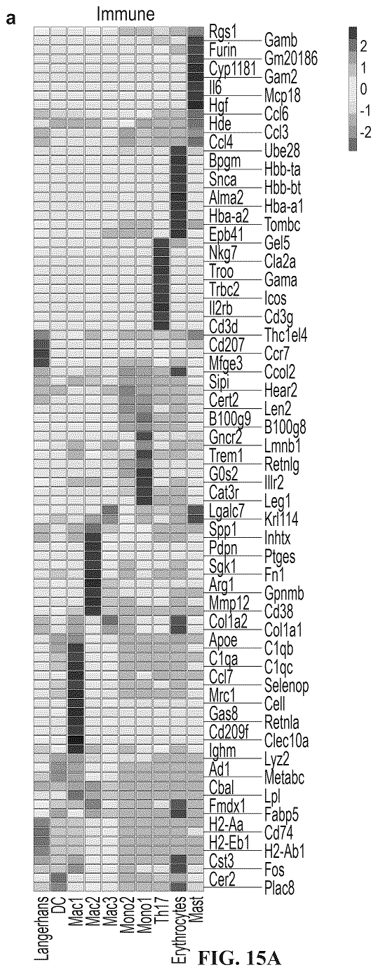

Since the CARD9 R101C mutation has been linked to dermatophytosis due to Trichophyton rubrum infection, the role of this mutation in the immune response to this fungal pathogen was investigated using a mouse model of dermatophytosis (Lanternier et al., 2013). Mice were injected intradermally with T. rubrum and analyzed 48 h post-infection. CARD9 R101C mice showed higher burden of fungal spores when compared to wild-type mice, mimicking the phenotype observed in CARD9^{-/-} mice (FIG. 5A). Histological analysis of the harvested skin demonstrated a well-defined pyogranuloma containing mostly neutrophils, focal dermal fibrosis and severe acanthosis in the skin of WT mice infected with T. rubrum. Formation of pyogranuloma and infiltration with immune cells was severely impaired in both CARD9^{-/-} and CARD9 R101C mice (FIG. 5B). More detailed analysis revealed no differences in the skin composition at the steady state between different genotypes. After infection, focal accumulation of large vacuolated macrophages, neovasculatization and mild focal acanthosis with underlying focal dermal granulation and fibrosis sporadically present in the skin of infected CARD9^{-/-} and CARD9 R101C mice (FIG. 5B). Analysis of the immune composition of the infected skin revealed severe impairment in immune response post-infection, with reduced infiltration of monocytes and neutrophils, as well as decreased secretion of the neutrophil chemoattractant, CXCL1 (FIGS. 5C-5F and FIG. 14A). Additionally, analysis at 9 days post intradermal infection demonstrated persistent infection in CARD9 R101C mice whereas live fungal organisms were not recoverable from WT mice (FIG. 5G). Histological analysis of infected skin revealed increased immune infiltration and presence of pyogranuloma in WT mice that was reduced in both CARD9^{-/-} and CARD9 R101C mice (FIG. 5H). Similar to what was observed for the early time point, at day 9 after the infection, CARD9 R101C showed impaired immune response demonstrated by lack of monocyte and neutrophil infiltration (FIGS. 5I-5L and FIG. 14A).

To further validate our findings, a foot pad model of dermatophytosis with transdermal injection of fungal spores into the foot pad was used. Similar to findings in the back skin infection model, CARD9 R101C mice injected with T. rubrum showed impaired clearance of spores and lack of neutrophil infiltration into the site of injection (FIGS. 14B-14D). The observed phenotype recapitulates the response observed in CARD9^{-/-} mice. Together, these data indicate that CARD9 R101 is essential for CARD9 activation and subsequent activation of the immune response against fungal pathogens. Taken together, R101C mice exhibited a similar phenotype to CARD9^{-/-}, which accurately models the severe impairment in antifungal immunity observed in human patients with this rare variant.

### Example 6: CARD9 R101C Disrupts Intercellular Coordination Through Dynamic Remodeling of Immune, Stromal, and Keratinocyte Cell State

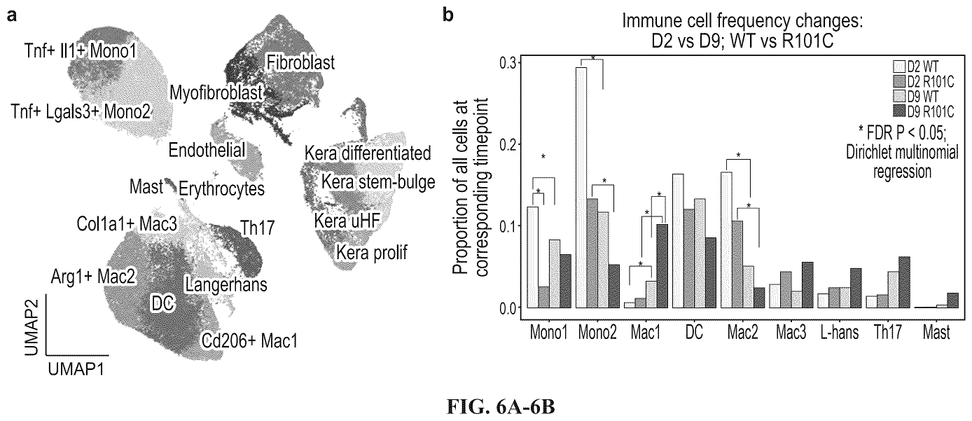

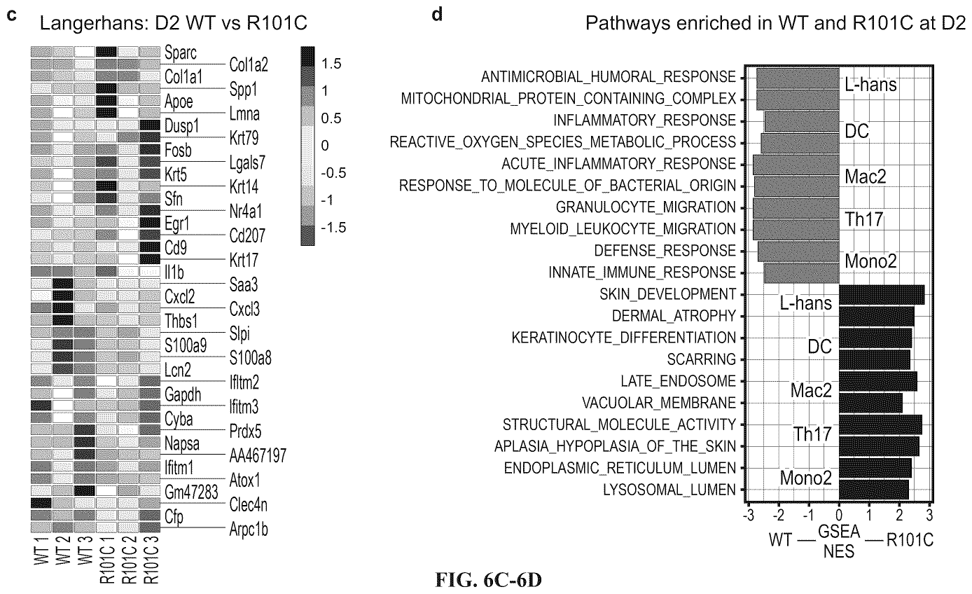

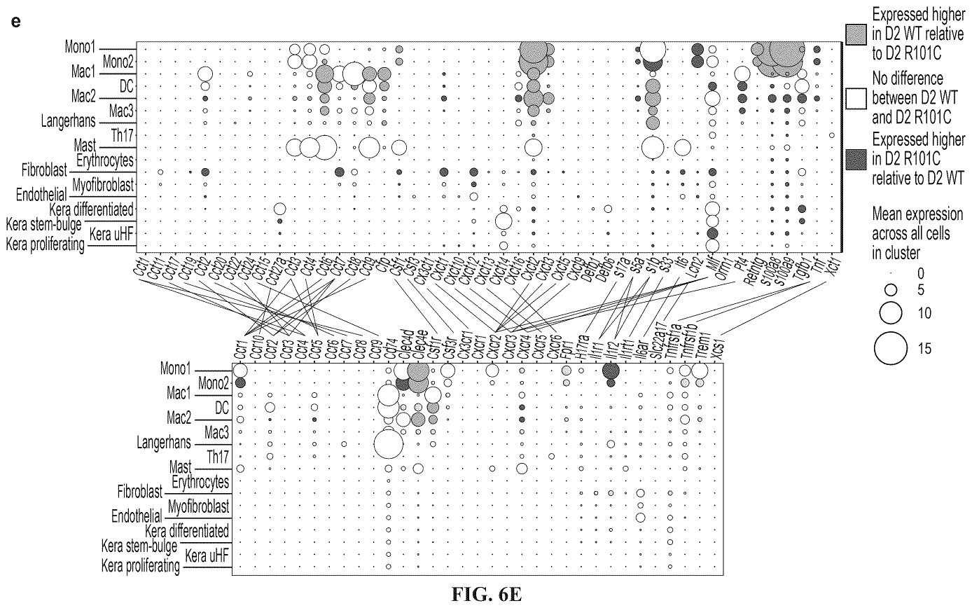

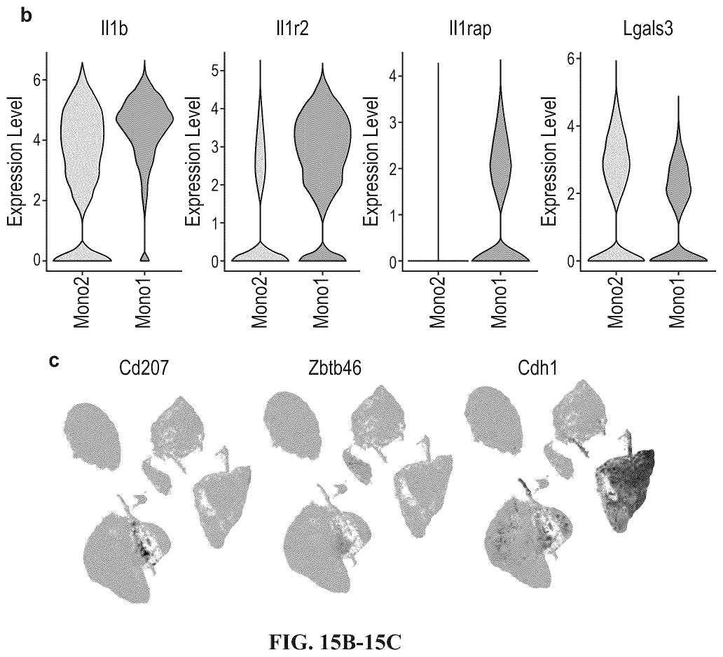

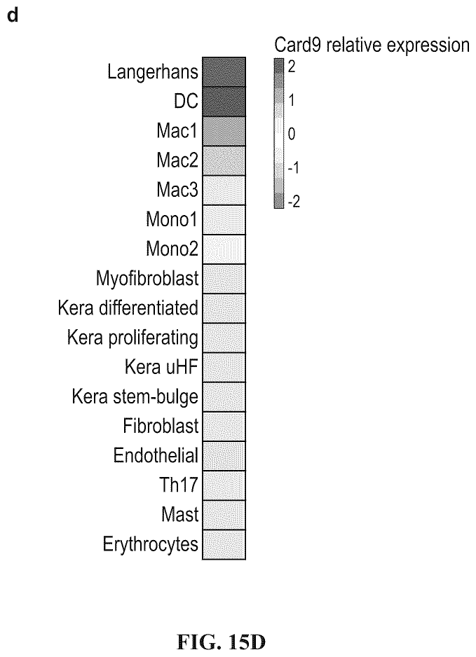

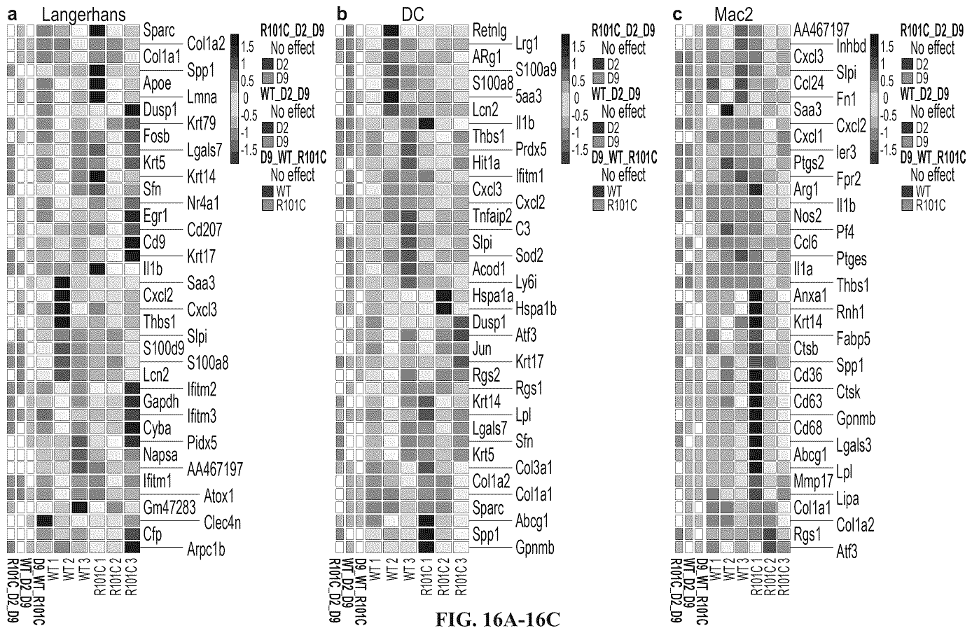

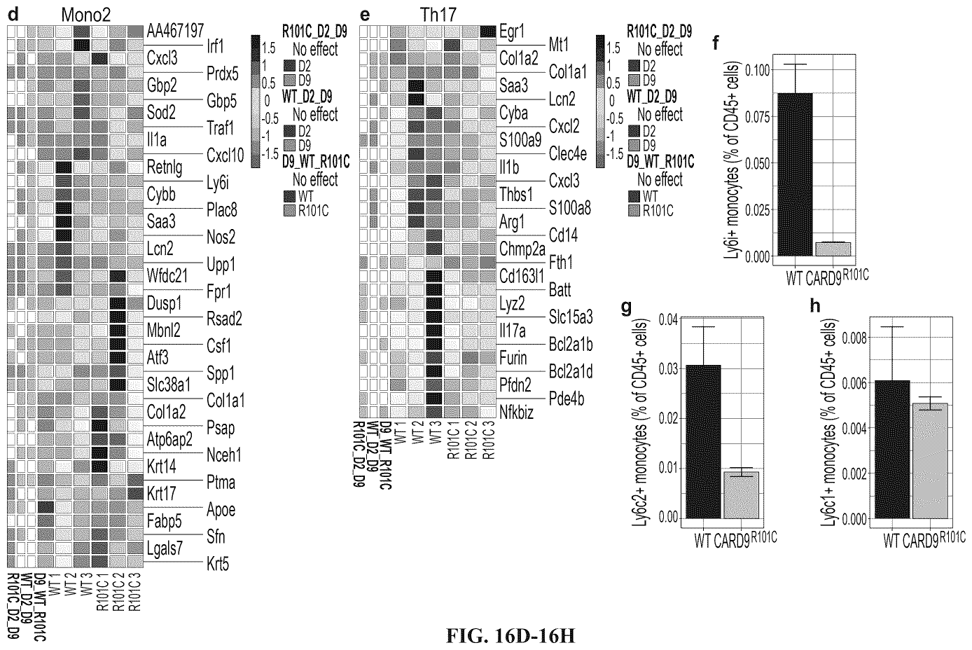

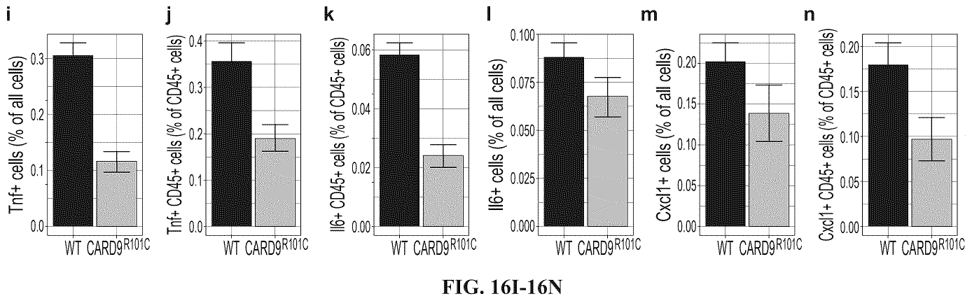

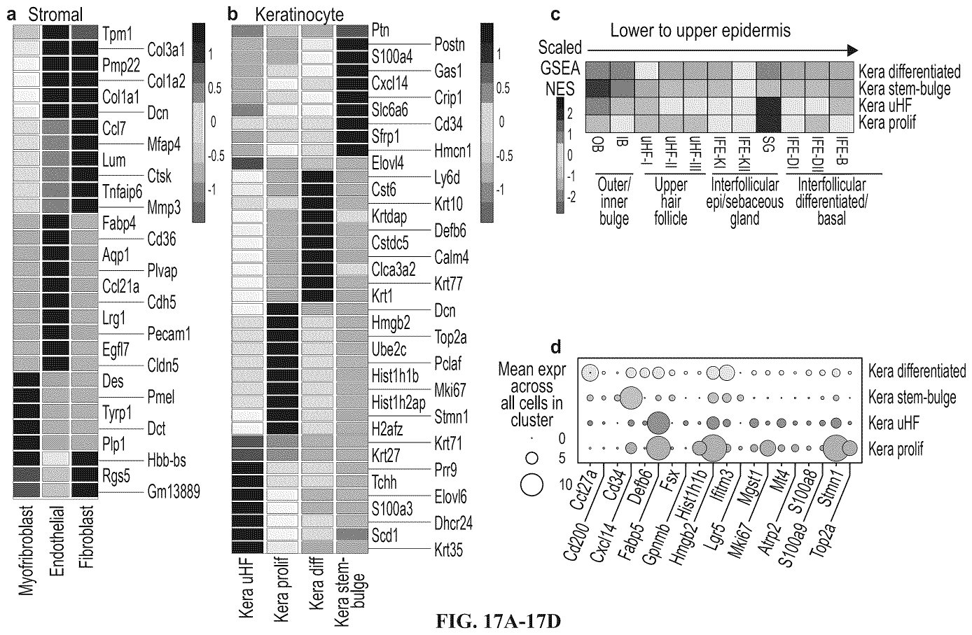

Given the divergent inflammatory responses to fungi observed between WT and CARD9 R101C mice, how CARD9 R101C remodels skin immunity and cell state was explored. In particular, an explanation was sought for the dichotomy between high fungal burden and reduced cellular infiltration and inflammation in CARD9 R101C mice. Thus, single cell RNA sequencing (scRNAseq) was performed of skin from WT and CARD9 R101C mice infected with T. rubrum at day two (D2) and day nine (D9) post-infection. In total, 75,647 cells were recovered, which yielded 17 clusters broadly partitioned into three compartments—immune cells, stromal cells, and keratinocytes, which broadly aligned with previously defined keratinocyte states (Joost et al., 2016) (FIG. 6A, FIGS. 15A-15C, FIGS. 17A-17D). Together, these data comprise a comprehensive skin cell atlas. The prevalence of myeloid cells, in particular of the I11b-high monocyte cluster 1 and Gal3-high monocyte cluster 2, were altered in CARD9 R101C skin (FIG. 6B and FIG. 15A-15B). As CARD9 expression was detected only in myeloid cells, and most highly expressed in Langerhans cells (FIGS. 15C-15D), the cell-autonomous effects of CARD9 on the skin immune response to fungi was investigated. Differential expression analysis between WT and R101C cells at D2 within each cluster revealed a broad reduction in expression of innate immune signaling genes in both myeloid cells and T cells. In contrast to R101C, WT cells exhibited upregulation of interferon-induced genes such as Ifitm2/3 and Gbp2/5, Il1a/b, chemokines such as Cxcl1/2/3, and Lcn2 across all myeloid populations and Th17 cells (FIGS. 6C-6D and FIGS. 16A-16E). In agreement with the flow cytometry data, a reduction in CD45 positive cells expressing Ly6C markers, Tnf, Il6 and Cxcl1 was observed in CARD9 R101C skin (FIGS. 16F-16N).

To define the cell-cell communication circuits driving impaired monocyte recruitment and broad loss of inflammation in CARD9 R101C mice, an extensive list of chemokines, cytokines, and their receptors was curated. Their expression was then investigated across all clusters, and whether their expression depends on CARD9 genotype at D2 post-infection (Mantovani et al., 2004). This analysis revealed a highly dynamic, CARD9-dependent network of interactions within and between specific immune, stromal, and keratinocyte clusters at the early time point of infection (FIG. 6E). Upon infection, WT myeloid cells produce inflammatory mediators such as Ill, Tnf, Cxcl2 and Cxcl3 that both lead to recruitment of monocytes and neutrophils to the infected tissue as well as activation of stroma and keratinocytes. In return, activated fibroblasts and keratinocytes amplify the immune response by secreting chemokines such as Cxcl1, Cxcl2, Cxcl3, Cxcl12, and Cxcl14. In particular, Cxcl2, Cxcl3 and Il1b were expressed most highly in Mono1 and were also upregulated in WT across most other clusters at D2, suggesting a key role in the antifungal immune response. Together, these data suggest that CARD9 disrupts positive feedback loops comprised of myeloid cells that recruit monocytes and other immune cells primarily through the Il1 and Cxcl2/Cxcr2 axes.

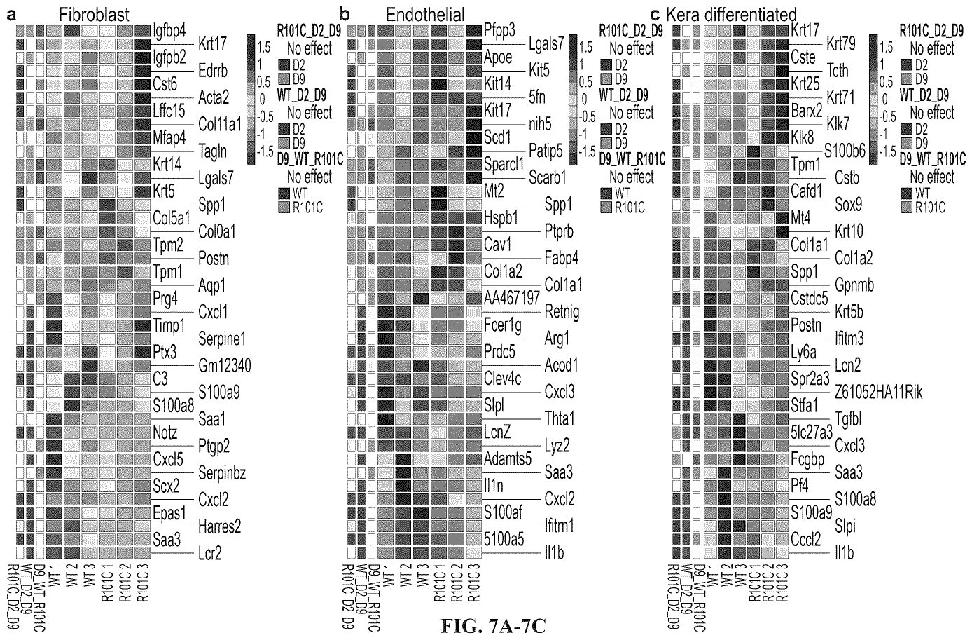

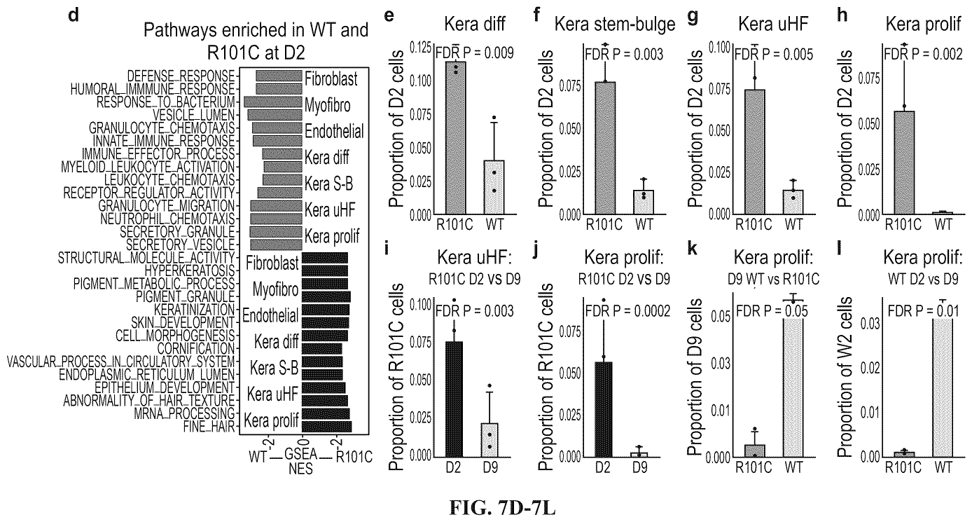

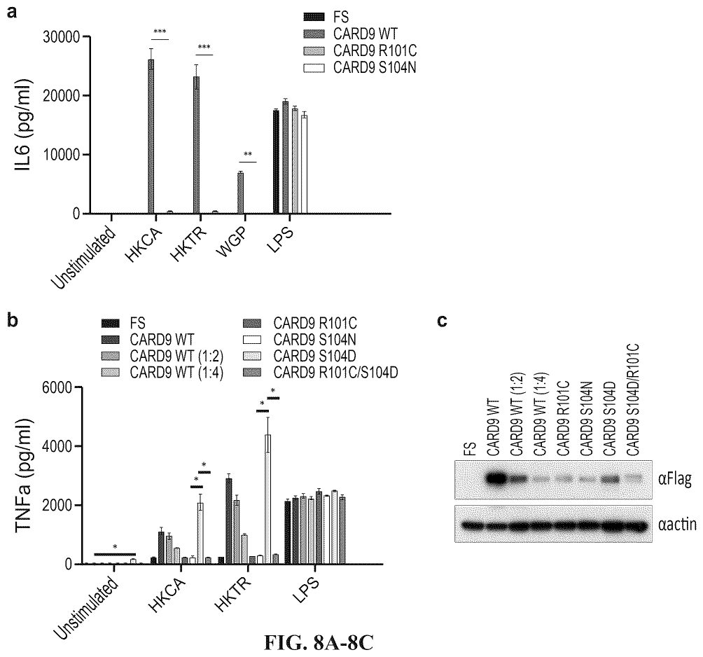

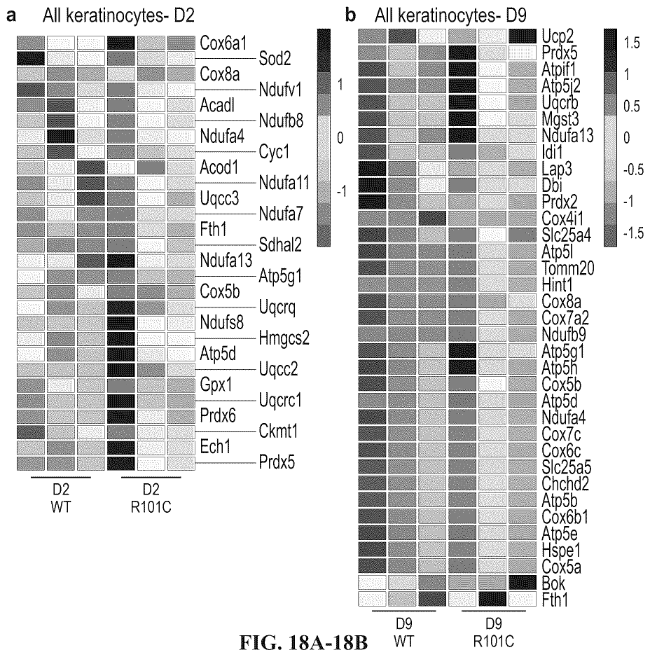

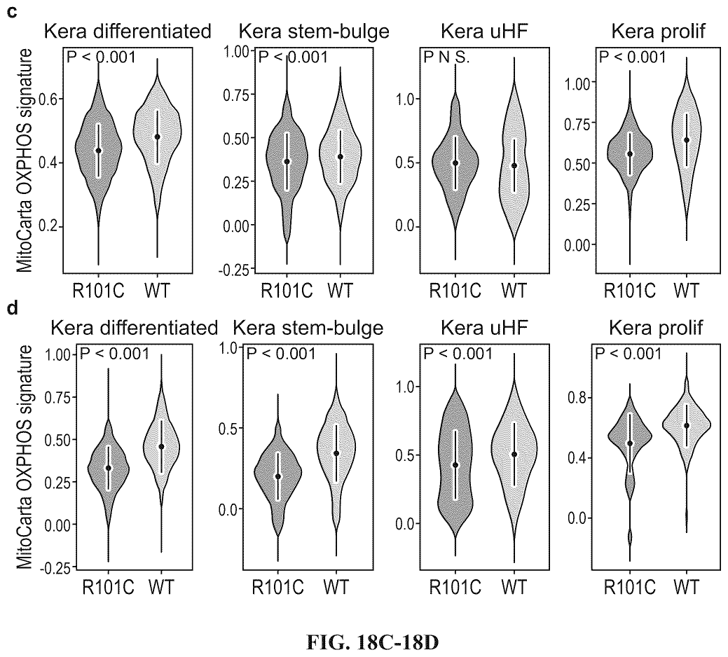

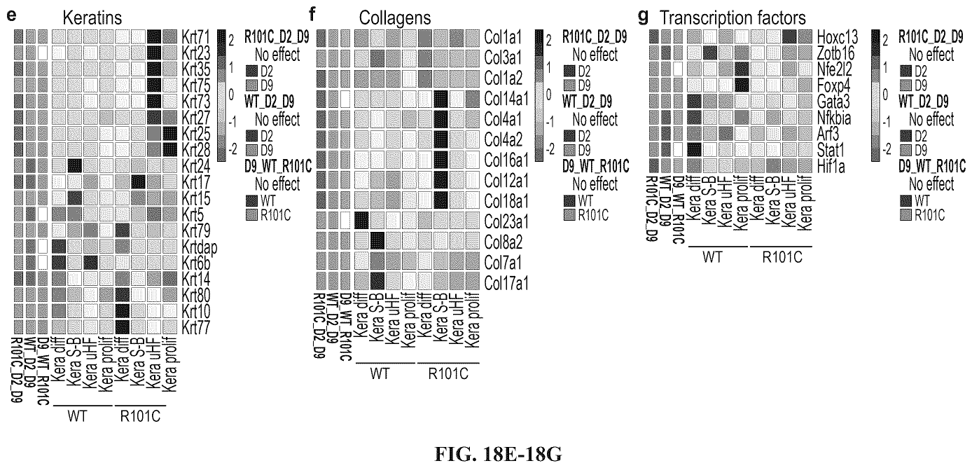

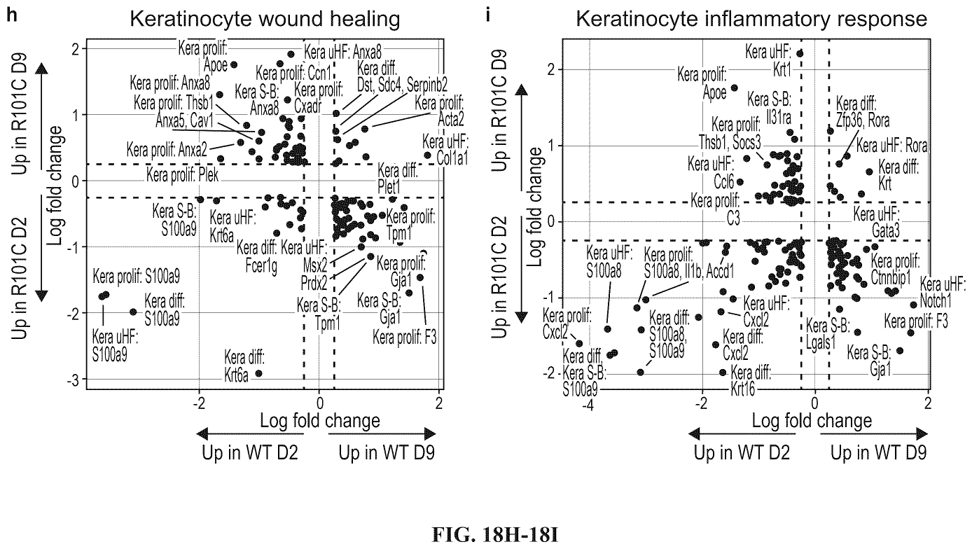

Despite the fact that stromal cells and keratinocytes do not express CARD9 (FIG. 14D), dramatic differential expression of chemokines and receptors in these cell types was observed in WT relative to R101C mice after infection, suggesting that CARD9 exerts widespread cell non-autonomous effects on tissue immunity (FIG. 6E). Consistent with the analysis of immune clusters, the top DEGs and pathways at D2 between WT and R101C cells in stromal and keratinocyte clusters were related to the innate immune response (FIGS. 7A-7D, FIGS. 16A-16D). GSEA analyses also indicated that oxidative stress and phosphorylation pathways were enriched in WT over CARD9 R101C keratinocytes (FIGS. 18A-18D), which may reflect overall cell stress due to ongoing strong immune response. Without intending to be bound by theory, these data indicate that it is likely that the primary cell non-autonomous consequence of CARD9 R101C is the disruption of gene expression programs related to innate immunity in stromal cells and keratinocytes.

To further study the consequences of impaired immune response in keratinocytes, the prevalence of each keratinocyte cluster in the atlas across CARD9 genotypes and timepoints was examined. At D2, all four keratinocyte clusters were expanded in skin from CARD9 R101C mice relative to WT mice (FIGS. 7E-7H). Moreover, at D9, both the Kera proliferating and Kera uHF clusters were strongly enriched in CARD9 R101C mice at D2 compared to D9 (FIGS. 71-7J). In contrast, the proliferative keratinocyte response was delayed in WT mice (FIGS. 7K-7L), suggesting that high inflammation in WT cells may delay wound healing. Together, these data suggest that CARD9 R101C promotes an early state-specific, proliferative keratinocyte response.

To identify changes in keratinocyte cell state independent of cluster prevalence, specific keratinocyte DEGs related to wound healing across timepoints was also examined. While some wound healing genes, such as the collagen and keratin family members were upregulated both at D2 and D9 in keratinocytes (FIGS. 18E-18I), others—such as the connexin Gja1 (Cx43), previously shown to regulate keratinocyte proliferation in skin wound healing—were induced early in R101C keratinocytes, but later in WT keratinocytes. Thus, the cell non-autonomous effects of CARD9 R101C include disruption of wound healing and inflammation in keratinocytes across multiple timepoints of infection.

Without intending to be bound by theory, these analyses suggest that CARD9 R101C disrupts fungal sensing, wound healing, and inflammatory responses, and thus may result in the perception of sterile tissue damage that elicits a distinct response in CARD9 R101C relative to WT. Without intending to be bound by theory, it was shown that through both cell autonomous and non-autonomous pathways, CARD9 R101C disrupts an intercellular communication network that amplifies chemokine-driven recruitment of effector immune cells essential for the resolution of the infection.

Naturally occurring genetic variants provide new opportunities to learn about protein functions and modes of regulation that would be challenging to uncover by directed mutagenesis. Immunodeficiencies associated with infectious diseases manifest in distinct phenotypes, making it easier to identify predisposing loss-of-function variants. CARD9 protein is a prime example of how rare variants can result in severe fungal infections (Ji et al., 2016). In order to identify new modes of regulation of anti-fungal responses, and in particular CARD9 signaling, known CARD9 genetic variants were analyzed. The R101C mutation located in the linker region of the protein was specifically focused on, based on the hypothesis from structural modeling that the linker may be crucial for CARD9 activation and CBM complex formation. Indeed, it was observed that introducing the R101C variant resulted in a loss-of-function phenotype in primary cells without affecting protein expression and stability. Additionally, R to C substitution at the 101 position was demonstrated to prevent phosphorylation of the neighboring S104 upon fungal stimulation, due to disrupting the minimal kinase recognition motif RXXS, which is essential for CARD9 activation.

Structural models indicate that CARD9 adopts an autoinhibited multimeric conformation. Truncated CARD9 containing CARD and CC1 domains adopts a dimeric form (Holliday et al., 2019) and it was observed that full length CARD9 with CARD, CC1, CC2, and C-terminal domain exists as a tetrameric dimer of dimers. The interface of the CARD domain with the linker and CC1 domain is mediated through hydrophobic interactions and aromatic packing, including multiple amino acids of both CARD and CC1 regions, with the linker acting as a key switch region (Holliday et al., 2019). Disruption of the interface frees the CARD domain, providing a platform for subsequent filament formation (Holliday et al., 2019). It was demonstrated that S104D phosphomimetic mutation accelerated Bcl10 filamentation in vitro, presumably by freeing the CARD domain. Importantly, S014D only induced Bcl10 filamentation when position 101 was R, suggesting that S104 phosphorylation and R101 operate together to activate CARD9.

Previous research has identified point mutations that can disrupt the CARD-CC1 interaction, leading to robust activation of NFκB in an overexpression system (Holliday et al., 2019). NMR structures of the CARD9 autoinhibited dimer depict the side chain of R101 located between the CARD and CC1 domains (Holliday et al., 2019). In this context, the results in the present disclosure suggest a model for how phosphorylation at S104 may cooperate with R101 to actuate a switch mechanism. Without intending to be bound by theory, S104 phosphorylation may initiate engagement of the negatively charged phosphate on S104 with the positively charged R101 sidechain, thus rotating R101 out of the CARD-CC1 interface and disrupting the autoinhibited conformation to allow for templating Bcl10 polymerization. Without intending to be bound by theory, alternatively, S104 phosphorylation and R101 may independently cooperate to promote activation through stabilization of CARD9 filaments. Given the observation that phosphomimetic S104D leads to activation of CARD9 only when position 101 is R, the former model is favored where R101 functions as a switch that is actuated by S104 phosphorylation. It is possible, therefore, to develop inhibitors that function as intermolecular glues to lock the autoinhibited CARD9 complex in the off-state, thus disabling the R101/S104 switch mechanism.

The results described herein indicate that the R101C variant disrupts the R101 switch mechanism triggered by S104 phosphorylation, which may be an initiating event that promotes filamentation, and the R70W variant acts at a later stage in the process preventing proper assembly of filaments. Another variant associated with fungal infection disrupts a critical phosphorylation site in CARD9 at position T231, which links CARD9 activation through PKCδ (Vaezi et al., 2018, Strasser et al., 2012). Without intending to be bound by theory, PKCδ may phosphorylate S104 to activate the R101 switch and T231 to promote dissociation of coiled coil domains within the autoinhibited multimer, together allowing for the assembly of CARD9 into filaments. Additional post-translational modifications on CARD9, such as ubiquitination on K125, may further promote templating Bcl10 polymerization by disruption of the autoinhibited CARD9 multimer, or perhaps more likely by stabilizing the assembled filaments and promoting recruitment of other signaling proteins necessary for NFkB activation (Cao et al., 2015).

Functional genetics can reveal mechanisms of CARD9 regulation and also provide an opportunity to connect those mechanisms to clinical phenotypes through deeper understanding of immune regulation in vivo. In order to better mimic the clinical phenotypes linked to CARD9 deficiencies, two distinct models were applied to study the R101C variant. Extreme susceptibility to systemic C. albicans infection with fungal overgrowth in brain and kidney was observed, as well as systemic inflammation, a phenotype that reflects clinical disease associated with multiple CARD9 variants. As the CARD9 R101C missense variant has been described in connection with deep dermatophytosis due to T. rubrum infection, a mouse model of dermatophytosis (Lanternier et al., 2013) was also used. In this model, CARD9 R101C is associated with lack of monocyte infiltration in the skin as well as reduced innate immune activation of CARD9-expressing myeloid cells. Furthermore, it was demonstrated that cell-intrinsic expression of CARD9 is not required for it to exert broad effects on skin cell states. The cell non-autonomous impact of CARD9 is exemplified by pronounced cell state changes (i.e. DEGs) and corruption of wound healing and resolution of inflammation.

CARD9-dependent cell non-autonomous effects on stromal cells and keratinocytes was observed, which express chemoattractants and cytokines essential for recruitment of immune cells into inflamed tissue sites. Indeed, recent studies have highlighted the importance of nonimmune cells in the regulation of all phases of the immune response (Nowarski et al., 2017). In the case of ulcerative colitis, Oncostatin M from myeloid cells acts on stromal cells to potentiate chemokine secretion in a manner that contributes to anti-TNFα treatment resistance (West et al., 2017). Along similar lines, abnormal activation of Il1 signaling circuits in the inflamed intestine drives neutrophil recruitment through a cellular network associated with resistance to anti-TNFα therapy (Friedrich et al., 2021). Both mechanisms provide insights into unique disease pathotypes that disrupt tissue homeostasis. Similarly, a complex cellular network in the skin is identified in the present disclosure that provides protective tissue immunity against fungal infection.

The results described herein above, were obtained using the following methods and materials.

### Mice

Mice were maintained in the specific-pathogen free facilities at Massachusetts General Hospital. All animal studies were conducted according to protocols and procedures approved by the Institutional Animal Care and Use Committee (IACUC) at Massachusetts General Hospital as per MGH IACUC protocols #2003N000158 and #2008N000078. Card9^{-/-} mice were described previously (Hara et al., 2007). Card9^{R101C} CRISPR knock-in mice were generated for the purpose of the studies described herein.

Candida albicans Culture and Infection

C. albicans (strain ATCC SC5314) was grown on yeast-peptone-dextrose (YPD; 1% yeast extract, 2% bactopeptone, 2% glucose, 2% agar) at 30° C. For each infection, a fresh colony was picked or inoculated directly from frozen stocks and grown in YPD broth over-night at 30° C. with agitation. Prior to infection, fungi were washed three times with PBS and counted using a LUNA™ Automated Cell Counter (Logos Biosystems, Annandale, VA) and diluted in PBS to 7.5×10⁵ yeast/ml. Each mouse was infected with 200 μl of the suspension (150,000 yeast) via lateral tail vein injection. Control mice were infected with PBS alone. Mice were monitored daily for the duration of the experiment as per MGH IACUC protocol #2008N000078. Mice were assessed based upon a 12-point scale to determine when an individual animal should be euthanized due to illness and removed from the study. This was developed in conjunction with the MGH IACUC and includes the following symptoms and points assigned to each in parentheses: hunched posture (3), ruffled and/or matted fur (3), shivering (3), abnormal breathing (increased respiratory rate) (12), 75% reduction in activity compared to controls (3), inactivity leading to inability to acquire food or water (12), and barrel rolling (12). If any animal reaches 12 points, they are humanely euthanized and counted as a death for the purposes of survival analysis.

Trichophyton rubrum culture and infection

T. rubrum (Clinical isolate was provided by Paul Verweij from Radboud University Medical Centre, The Netherlands) was plated on BD Difco Sabouraud dextrose agar (SDA) (Fisher Scientific #BD210950) and kept in room temperature in a humid chamber for up to 1 month to allow the sporulation. For spore isolation, culture plate was incubated for 5 min with PBS with 0.05% Triton X-100 scraped and passed through 100 μm cell strainer. Suspension was centrifuged for 10 min at 2000 rpm, washed 1× with PBS, manually counted using Neubauer counting chamber and resuspended in PBS to the desired concentration. For the back skin infection, back skin was shaved and 50 μl of spore suspension was injected intradermally into 4 separate spots. Skin was also abrased with pumice stone and 100 μl of spore suspension was applied topically. For footpad injection, 50 μl of spore suspension was injected into the left back foot pad. Experiments were performed according to MGH IACUC protocol #2003N000158. For preparation of heat killed T. rubrum (HKTR), spore suspension was incubated at 95° C. for 30 min.

### Quantification of Fungal Burden

For determination of fungal burdens after infection, mice were euthanized with CO₂ and kidneys and brain were harvested and placed into conical tubes containing sterile PBS. Tubes were weighed before and after addition of the organ to determine the organ weight. Organs were homogenized using an immersion homogenizer (Omni TH, Omni International, Kennesaw, GA) and serial dilutions of the homogenate were spread onto YPD plates. Plates were incubated 24-48 hours at 30° C. until visible colonies formed. Colony forming units (CFU) were counted and CFU/g of tissue was calculated for each organ. For T. rubrum, back or foot pad skin was harvested with sterile tools and placed in 1 ml of PBS. Tissue was dissociated with a mechanical homogenizer. Serial dilutions were performed and plated on Remel Dermatophyte test medium (Thermo Fisher Scientific #R01365). Plates were incubated over 5 days at RT in a humid chamber and CFU was counted. Data is represented as CFU/g of harvested tissue.

### Immunophenotyping

Single-cell suspension for flow cytometry analysis from spleen and thymus was prepared by disrupting tissues between the frosted ends of two microscope slides using Staining Buffer. Bone marrow single-cell suspension was prepared by flushing femur and tibia. Red Blood Cell (RBC) lysis was performed using 1×RBC Lysis Buffer. Kidney tissue was cut into small pieces with scissors and incubated for 20 min in digestion solution containing Liberase (Roche #385040) and DNAse I (Roche #10104159001) at 37° C. with agitation. After the digestion, samples were placed on ice and the reaction was stopped using stop buffer containing 5% FBS and 1 mM EDTA. Cells were centrifuged 5 min 1500 rpm at 4° C. and washed 1× with PBS. Cells were passed through a 100 uM cell strainer. Brain tissue was processed as previously described (Lionakis, M. S. et al., 2011). Briefly, tissue was homogenized using syringe plunger and 100 mM filter on ice. Suspension was separated using Percoll gradient and leukocyte interphase was collected after centrifugation at 2450 rpm for 20 min at 4 C. Cells were stained using appropriate antibody cocktail mentioned in the following section for 1 h on ice, washed and run on CytoFLEX LX flow cytometer (Beckman Coulter) or Cytek Aurora (Cytek Biosciences). Isolated single cells were filtered over 40-pM filter and re-suspended in FACS buffer followed by flow cytometry analysis.

Three multi-color flow cytometry panels for CytoFLEX FL were developed to assess major myeloid, lymphoid subsets in the following organs: spleen, thymus and bone marrow comparing controls with the genetically modified mouse models. The following antibodies were used for a 16-color flow cytometry spleen panel: AF488-CD19 (clone 6D5), PE/DZ594-CD23 (clone B3B4), BV421-CD21/35 (clone 7E9), BUV395-CD93 (clone 493), BV785-CD11b (clone M1/70), APC Cy7-F4.80 (clone BM8), AF647-Ly6G (clone 1A8), BV650-CD11c (clone HL3), PE Cy7-CD3e (clone 145-2C11), PE Cy5-CD122 (clone 5H4), BV510-CD8α (clone 53-6.7), PE-CD4 (clone H129.19), AF700-CD45 (clone 30-F11), BV605-IA/IE (clone M5/114.15.2), BUV661-CD5 (clone 53-7.3) and ViaKrome Live/Dead dye (Beckman Coulter, #C36628).

The following antibodies were used for a 10-color flow cytometry thymus panel: BUV395-CD4 (clone GK1.5), BV510-CD8u (clone 53-6.7), AF700-CD45 (clone 30-F11), PacBlu-CD25 (clone PC61), PE-CD44 (clone IM7), APC Cy7-CD117 (clone 2B8), PE Cy7-γδTCR (clone GL3), APC-TCRβ (clone H57-597), lineage cocktail includes: FITC-NK1.1 (clone PK136) & FITC-TER119 (clone TER-119) & AF488-CD19 (clone 6D5) and ViaKrome Live/Dead dye (Beckman Coulter, #C36628).

The 13-color flow cytometry bone marrow panel includes the following antibodies: BV605-B220 (clone RA3-6B2), PE Cy7-CD43 (clone S11), PE-CD24 (clone 30-F1), APC-BP1 (clone 6C3), BV421-IgM (clone RMM-1), BUV395-IgD (clone 11-26), BV650-CD150 (clone TC15-12F12.2), AF700-CD48 (clone HM48-1), PE Cy5-CD34 (clone MEC14.7), FITC-CD16/32 (clone 93), BV785-SCA1 (clone Ly-6A/E), APC Cy7-CD117 (clone 2B8) and ViaKrome Live/Dead dye (Beckman Coulter, #C36628).

One multi-color flow cytometry panels for Cytek Aurora were developed to assess major myeloid and lymphoid subsets in the following organs: spleen, kidney and brain, comparing controls with the genetically modified mouse models. The following antibodies were used for a 22-color flow cytometry spleen panel: Zombie UV Live/Dead (BioLegend, #423108), BV711-F4/80 (clone BM8), BUV395-CD4 (clone GK1.5), BV480-CD117 (clone 2B8), BUV496-CD123 (clone 5B111), APC Cy7-TCRB (clone H57-597), BV570-CD90.2 (clone 30-H12), PE CF594-Siglec-F (clone E50-2440), BV650-CD11c (clone N418), PE Cy5-CD3 (clone 145-2C11), PE-CD103 (clone 2E7), eFluor450-Ly6C (clone HK1.4), BV605-Ly6G (clone 1A8), BUV805-CD45 (clone 30-F11), BV510-CD8a (clone 53-6.7), PE Cy7-CD64 (clone X54-5/7.1), BUV737-CD19 (clone 1D3), AF700-CD8b (clone YTS156.7.7), BV785-CD11b (cloneM1/70), AF563-IgA, BUV563-IA/IE (clone M5/114.15.12) and FITC-EpCAM (clone G8.8).

### Histology

Tissue was harvested into the Tissue-Tec cassettes (#4118-01) and fixed in 10% neutral buffered formalin, trimmed, processed routinely, embedded in paraffin, and stained with hematoxylin and eosin (H&E) and Periodic Acid-Schiff (PAS). Histology was performed by Tufts Comparative Medicine Services at Tufts University, Boston, MA. Histopathology was evaluated by a board certified veterinary pathologist.

### Isolation of Spleen CD11c+ Cells

Single cell splenic suspension prepared as described above was incubated with biotin anti-mouse-CD11c antibody (BioLegend #117304, clone #N418) for 20 min on ice. Cells were washed 1× with MACS buffer (PBS pH 7.2, 0.5% BSA, 2 mM EDTA) and incubated with anti-biotin MicroBeads (Miltenyi Biotec #130-090-485) according to product instructions. Cells were positively selected using MACS cell separation MS columns (Miltenyi Biotec #130-042-201) according to the manufacturers instruction. Selected cells were centrifuged, washed 1x with PBS and processed for Western Blotting.

Preparation of Single Cell Suspension from Back Skin

Skin was harvested with sterile tools, cut into small pieces with scissors and incubated for 1.5 h in digestion solution containing Liberase (Roche #385040) and DNAse I (Roche #10104159001) at 37° C. with agitation. After the digestion, samples were placed on ice and the reaction was stopped using stop buffer containing 5% FBS and 1 mM EDTA. Cells were centrifuged 5 min 1500 rpm at 4° C. and washed 1x with PBS. Cells were passed through a 100 uM cell strainer and counted.

For the flow cytometry analysis, the following 9-color panel was used: BV785-CD45 (clone 30-F11), AF488-F4/80 (cloneBM8), AF647-CD64 (clone X54-5/7.1), BV650-Ly-6G (clone 1 A8), PerCP-Cy5.5-Ly-6C (clone HK1.4), BV421-CD11b (cloneM1/70), PE-Cy7-CD11c (clone N418), AF700-I-A/I-E (clone M5/114.15.2) and ViaKrome Live/Dead dye (Beckman Coulter, #C36628). Samples were analyzed on CytoFLEX LX flow cytometer (Beckman Coulter) or Cytek Aurora flow cytometer (Cytek Biosciences).

### CXCL1 ELISA

Back or foot pad skin was harvested with sterile tools and placed in 1 ml of PBS. Tissue was homogenized by mechanical disruption and centrifuged. Supernatant was processed for CXCL1 ELISA (Thermo Fisher Scientific #EMCXCL1) according to manufacturers' protocol.

### Cell Culture

HEK293T cells were maintained at 37° C. and 5% CO₂ in Gibco DMEM (Thermo Fisher Scientific #10569044) supplemented with 10% fetal calf serum and 15 μg/ml gentamycin sulfate.

### Mouse Bone Marrow Derived Dendritic Cell Culture

To generate BMDCs and BMDMs, murine bone marrow cells obtained from murine femur and tibia were cultured for 7 days in complete RPMI with glutamax (Life Technologies #72400-120) supplemented with 10% FBS, 15 μg/ml gentamycin sulfate and 37.5 ng/ml recombinant murine GM-CSF (PeproTech #315-03) (for BMDCs) or 25 ng/ml M-CSF (PeproTrch #315-02) and 5 ng/ml IL-3 (PeproTech #213-13) (for BMDMs) at 37° C. and 5% CO₂. Media was changed at day 3 and fresh media was added at day 5 of the culture. Cells were used for functional assays on day 8.

### Bone Marrow Monocytes Isolation

Monocytes from bone marrow were isolated using monocyte isolation kit (Miltenyi Biotech #130-100-629) and LS columns (Miltenyi Biotech #130-042-401) according to the manufacturer's instruction. Isolated cells were plated in 96-well plate and stimulated O/N at 37° C. and 5% CO₂.

### Lentivirus Production and BMDC and BMDMs Transduction

All constructs used were cloned into pCDH-CMV backbone (Addgene #72265). For virus preparation and BMDC or BMDMs infection, protocols from the Broad Institute's RNAi Consortium shRNA Library were used (www.broadinstitute.org/mai/trc/lib). In brief, 70% confluent HEK293T cells were transfected using Lipofectamine 3000 reagent (Thermo Fisher Scientific #L3000-015) to generate viruses with desired constructs. Media containing viruses was harvested 48 h post transfection, filtered through 22 μM cell filters and supplemented with 8 μg/ml of polybrene. BMDCs or BMDMs used for infection were grown in 12-well plates. On day 2 post isolation, media was aspirated and 2 ml of media containing virus was added per each well. Plates were centrifuged for 1.5 h at 2250 rpm at 30° C. After spinning down, the virus-containing media was removed and replaced with fresh RPMI supplemented with GM-CSF (BMDCs) or M-CFS and IL-3 (BMDMs). 2 days post transduction, fresh RPMI supplemented with 5 μg/ml puromycin was added to the wells. Functional assays were performed 3-4 days later. In experiments involving reconstitution of CARD9 in CARD9^{-/-} BMDCs or BMDMs, puromycin selection was omitted. Sequences of all guides used for CRISPR KO are listed in Table 2.

TABLE 2
sgRNA Guides Used for CRIPSR KO

| | | SEQ | | |
|---|---|---|---|---|
| | Target | ID | | |
| sgRNA | Gene | NO: | Target Sequence | |
| Akt1 | AKT1 | 12 | TACTTCCTCCTCAAGAACGA | |
| sg1 | | | | |
| Akt1 | AKT1 | 13 | CTCAGGCGTTTCCACATGGA | |
| sg2 | | | | |
| Camk | Camk | 14 | GGTGGTGCATCGTGACCTGA | |
| 2asg1 | 2a | | | |
| Camk | Camk | 15 | GATATACAGGATGACGCCTA | |
| 2asg2 | 2a | | | |
| Camk | Camk | 16 | ACTTCACGACAGTATATCGG | |
| 2dsg1 | 2d | | | |
| Camk | Camk | 17 | ATTACATACATCGTAAGCTC | |
| 2dsg2 | 2d | | | |
| Clk3 | Clk3 | 18 | ATGATCGTATACCCTACCAG | |
| sg1 | | | | |
| Clk3 | Clk3 | 19 | AGGCGATGCACATATGACCA | |
| sg2 | | | | |
| pak2 | PAK2 | 20 | TAATGGAGTACCTCGCTGGT | |
| sg3 | | | | |
| pak2 | PAK2 | 21 | TATCTCTGCAGATTTACACA | |
| sg4 | | | | |
| Map2 | Map2 | 22 | CATTCTAGTGAACTCACGTG | |
| k1sg1 | k1 | | | |
| Map2 | Map2 | 23 | TGCAACTCCCCGTACATCGT | |
| k1sg2 | k1 | | | |
| Map2 | Map2 | 24 | CATCCTGGTGAACTCTCGCG | |
| k2sg1 | k2 | | | |
| Map2 | Map2 | 25 | GTGCAACTCGCCCTACATCG | |
| k2sg2 | k2 | | | |
| Map2 | Map2 | 26 | AAAGTGCGGCATGCTCAGAG | |
| k3sg1 | k3 | | | |
| Map2 | Map2 | 27 | TCCGGGAGTCCAGGTTCCGG | |
| k3sg2 | k3 | | | |
| Map2 | Map2 | 28 | TGGATTTGGATGTAGTAATG | |
| k4sg1 | k4 | | | |
| Map2 | Map2 | 29 | AAATGGTCCACAAACCAAGT | |
| k4sg2 | k4 | | | |
| Mapkap | Map | 30 | AGAGGCTGCAGAGATAATGC | |
| k3sg1 | kapk3 | | | |
| Mapkap | kapMap | 31 | ACTTCTGGCCAGAGCGCCGA | |
| k3sg2 | k3 | | | |
| Pak2 | PAK2 | 32 | GGTGTGCTCAAAATCAGATG | |
| sg1 | | | | |
| Pak2 | PAK2 | 33 | TACCAGGAGGAGTGAAACTC | |
| sg2 | | | | |
| PRKACA | PRKACA | 34 | CTTGCTCTAGGGCTACAACA | |
| sg1 | | | | |
| PRKACA | PRKACA | 35 | CAGGGAGTGCAGATACTCAA | |
| sg2 | | | | |
| PRKCA | PRKCA | 36 | GTGGATAAGTCCATAGAGCA | |
| sg1 | | | | |
| PRKCA | PRKCA | 37 | AACCACAAAACAGCAAACTG | |
| sg2 | | | | |
| PRKCB | PRKCB | 38 | AGTGTTCCCTCAATCCAGAG | |
| sg1 | | | | |
| PRKCB | PRKCB | 39 | AGGTAGGACTGGAGTACGTG | |
| sg2 | | | | |
| PRKCD | PRKCD | 40 | AGAAGGTGGCGATAAACTCG | |
| sg1 | | | | |
| PRKCD | PRKCD | 41 | AGCCCACCATGTATCCTGAG | |
| sg2 | | | | |
| Rock2 | Rock2 | 42 | TCCCGACTACATATCGCCCG | |
| sg1 | | | | |
| Rock2 | Rock2 | 43 | AGCACGGTTAAGAAAAACCC | |
| sg2 | | | | |
| RPS6KA | RPS6K | 44 | AAGGGGGCTTGATCTCACGC | |
| 1sg1 | A1 | | | |
| RPS6KA | RPS6K | 45 | AGGACCACCAGTCTGCACTG | |
| 1sg2 | A1 | | | |
| RPS6KA | RPS6K | 46 | CCAGAAGTAGTTAACCGCAG | |
| 3sg1 | A3 | | | |
| RPS6KA | RPS6K | 47 | CCAGTTGCAGGCTTAAAAGG | |
| 3sg2 | A3 | | | |
| RPS6KA | RPS6K | 48 | GCGGGAGCATGAACGCAAAG | |
| 6sg1 | A6 | | | |
| RPS6KA | RPS6K | 49 | CCTGAAGTAGTAAACAGACG | |
| 6sg1 | A6 | | | |
| EGFP | EGFP | 50 | GGGCGAGGAGCT | non- |
| sg1 | | | GTTCACCG | target |
| | | | | sgRNA |
| | | | | control |
| EGFP | EGFP | 51 | GAGCTGGACGGC | non- |
| sg2 | | | GACGTAAA | target |
| | | | | sgRNA |
| | | | | control |

### CRISPR Using RNP

BMDCs were nucleofected following the protocol from Freund et al., 2020. Briefly, sgRNA (Table 2, custom made from IDT) and Cas9 (IDT #1081059) were mixed and incubated for 20 min at RT. Following that, isolated bone marrow cells (2e6/reaction) were mixed with the sgRNA/Cas9 complexes and nucleofected using P3 Primary Cell 4D-Nucleofactor X kit S (Lonza #V4XP-3032) and program CM 137 in Lonza 4D-Nucleofector System. Following nucleofection, cells were plated in 12-well plates in RPMI media supplemented with GM-CSF and processed for analysis on day 8.

Heat-Killed Candida albicans Preparation

For each preparation, a fresh colony was picked and grown in BD Difco YPD broth (Thermo Fisher Scientific #DF0428-07-7) overnight at 37° C. with agitation. Fungi were centrifuged for 10 min at 2000 rpm, washed 1× with PBS and counted. Concentration was adjusted to 10⁹/ml in PBS and fungi were heat-killed by incubation at 95° C. for 30 min.

### BMDCs or BMDMs Stimulation

Mature BMDCs or BMDMs were harvested and plated in 96-well plates at a concentration of 10\hat{ }5/cells per well. The following day, media was replaced with media containing either HKCA (MOI 1:10), HKTR (MOI 1:10), WGP (50 μg/ml)(Invivogen #tltl-wgp) or LPS (10 ng/ml) (Invivogen #tlrl-peklps) and incubated for 24h. For p-p65 cells were stimulated for 15 min.

### Cytometric Bead Array

Cytokine concentration in serum or cell culture supernatants was assessed using BD Cytometric Bead Array (CBA) Mouse/Rat Soluble Protein Kit according to the manufacturer's instruction.

### Immunoprecipitation and Western Blotting

Mature BMDCs were harvested and 3×10\hat{ }7 cells were plated per 15 cm dish. The following day HKCA (MOI 1:10) was added to the media for 30 min at 37° C. In experiments with PKCdi 10 uM Sotrastaurin (Selleckchem #S2791) was added 1 h before stimulation with HKCA. Cells were harvested and washed 1× with PBS. For CARD9 IP to detect pS104-CARD9, the cell pellet was resuspended in 100 μl of lysis buffer (50 mM Tris-HCl pH 7.5, 150 mM NaCl, 0.5 mM EDTA, 1% NP-40) supplemented with 1× Halt Protease & Phosphatase Inhibitor Single-Use Cocktail (Thermo Fisher Scientific #78442) and Pierce Universal Nuclease (Thermo Fisher Scientific #88701) and kept on ice for 30 min. SDS was then added to the tube to a final concentration of 1% and lysates were heated at 95° C. for 10 min. Denatured cell lysate was placed on ice and diluted with standard lysis buffer to dilute SDS concentration to 0.01%. Lysate was incubated for 30 min and centrifuged at 2000 rpm for 10 min at 4° C. 50 μl of the supernatant was collected as whole cell lysate (WCL) fraction. Remaining supernatant was collected and incubated overnight with 10 al/sample of CARD9 antibody (Cell Signaling Technology #12283S) at 4° C. while rotating. The next day, 50 μl of Dynabeads Protein G beads (Life Technologies #10004D) were added and incubated for 1 h rotating at 4° C. Immunoprecipitates were washed 3× with standard lysis buffer and resuspended in 25 μl of 2× Laemmli buffer (BioRad #1610737) supplemented with 5% R-mercaptoethanol followed by boiling for 10 min at 75° C. For Bcl10 IP to detect CBM complex formation, cell pellets were resuspended in lysis buffer (PBS, 0.5% NP-40) supplemented with 1× Halt Protease & Phosphatase Inhibitor Single-Use Cocktail and Pierce Universal Nuclease and kept on ice for 10 min. Lysate was centrifuged for 10 min at 2000 rpm at 4° C. 50 μl of the supernatant was collected as whole cell lysate (WCL) fraction. Remaining supernatant was incubated overnight with 200 al/sample of Bcl10 antibody (Santa Cruz Biotechnology #sc-5273) at 4° C. rotating. The next day, 50 μl of Dynabeads Protein G beads were added and incubated for 1 h rotating at 4° C. Immunoprecipitates were washed 3× with standard lysis buffer and resuspended in 25 μl of 2× Laemmli buffer supplemented with 5% p-mercaptoethanol followed by boiling for 10 min at 75° C. Immunoprecipitates and WCL samples were resolved by SDS-PAGE using Bio-Rad Mini-PROTEAN TGX Stain-Free Gels and Boston Bioproducts SDS-Running buffer, transferred to Immobilon-P membranes (Millipore Sigma #IPVH08130) in Boston Bioproducts Transfer buffer. Membranes were blocked for 1 h in 5% milk in TBS-T and immunoblotted with the indicated antibodies overnight in TBS-T supplemented with 5% BSA. Detection was performed by enhanced chemiluminescence with the Western Lightning Chemiluminescence Reagent (Perkin Elmer Life Sciences #NEL104001EA) followed by blotting with the secondary HRP-conjugated antibodies (Daco). The following antibodies were used for Western Blotting: CARD9 (clone A-8 Santa Cruz Biotechnology #sc-374569), p-p65 (Cell Signaling #3033S), p65 (Cell Signaling #8242S), Bactin-HRP (Cell Signaling #5125S), Flag M2 (Sigma Milipore #F1894), pTXR (Cell Signaling #2351S).

### P65 Nuclear Translocation Assay

Cells were plated in 96-well plate, suitable for fluorescent microscope (PerkinElmer #6055302). The following day cells were stimulated for 30 min and processed for fluorescent staining. Cells were washed 3× with PBS and fixed 20 min in 4% PFA (Thermo Scientific #J19943-K2) at RT. Following 3× washes with PBS, cells were permeabilized using 0.5% Triton X-100 and 2% BSA in PBS for 1 h at RT. Cells were washed 3× with PBS and incubated O/N at 4° C. with anti-p65 antibody (1:100, Santa Cruz Biotechnology clone F-6 #sc-8008) in 1% BSA solution. The following day cells were washed 3× with PBS and incubated for 1 h in RT with secondary 488-conjugated anti-mouse antibody and 1× Hoechst 33342 (Life Technologies #H3570) in 1% BSA solution. Cells were imaged using Perkin Elmer Opera Phenix Imaging System and analyzed using Harmony Software.

### In Vitro Phosphorylation Assay

70% confluent HEK293T cells were transfected using Lipofectamine 3000 reagent (Thermo Fisher Scientific #L3000-015) to overexpress CARD9 variants. The following day cells were lysed using RIPA buffer (Boston BioProducts #BP-115) supplemented with 1× Halt Protease & Phosphatase Inhibitor Single-Use Cocktail and Pierce Universal Nuclease and kept on ice for 10 min. Lysate was centrifuged for 10 min at 14000 rpm at 4° C. Supernatant was incubated for 1 h with MagStrep XT beads (Fisher Scientific #NC0776437) at 4° C., rotating. Following 2× washes with RIPA buffer and 2× washes with PBS, samples were split in 2 and processed for in vitro phosphorylation using PKCdc Kinase Enzyme System (Promega #V3401) following manufacturer instruction. ATP was used to control the reaction. After 1h, reaction was stopped by adding 2× Laemmli buffer containing 5% β-mercaptoethanol and boiling for 10 min at 75° C.

pS104—Card9 Antibody Generation

Peptides and antibodies were developed by Vivitide (formerly known as New England Peptide).

### Peptide Synthesis:

Peptides were made using standard Fmoc solid-state peptide synthesis and purified using high-performance liquid chromatography (HPLC) to >95% purity as determined by HPLC and/or L C/MS.

| | | SEQ | |
|---|---|---|---|
| | | ID | |
| Name | Lot # | NO: | Sequence |
| Card9 | 3702-2 | 52 | Ac-CEPARVF |
| pS104 | | | (pS)MIIDAS- |
| | | | amide |
| Card9 | LP01491 | 53 | Ac-CEPARVFS |
| S104 NP | | | MIIDAS-amide |
| Card9 | LP01490 | 54 | H2N-EPACVF |
| pS104 | | | (pS)MIIDAS- |
| R101C | | | amide |

### Preparation of Immunogen (Maleimide):

Peptide 3702-2 above was conjugated to Sulfo-SMCC (Thermo/Fisher #22322) activated KLH (Thermo/Fisher #77600) through the sulfhydryl side chain of the added terminal Cysteine, then emulsified 1:1 (volume) with CFA/IFA.

### Immunization Protocol:

50-100 μg of immunogen was administered via intraperitoneal injection using the Freund's adjuvant system. The immunizations were scheduled as follows:
Day 0 Pre-immune+Boost (100 μg immunogen with CFA)
Day 14 Boost (50 μg immunogen with IFA)
Day 28 Boost (50 μg immunogen with IFA)
Day 42 Boost (50 μg immunogen with IFA)
Day 49 Tail Bleed

### Animals:

3× Balb/c mice (8-12 weeks old) were immunized, with animal protocols approved by vivitide's Animal Care and Use committee.

### Preparation of Screening Materials (Maleimide):

Peptides were conjugated to Sulfo-EMCS (Thermo/Fisher #22307) activated BSA (Thermo/Fisher #77110) through the sulfhydryl side chain of the added terminal Cysteine.

### Preparation of Screening Materials (Amine):

The peptide was conjugated to BSA (Thermo/Fisher #77110) through the N-terminal free amine with bi-functional amine linker glutaraldehyde.

### Fusion of Spleen Cells:

Mouse 2 was euthanized, and the spleen excised. The spleen cells were dissociated and fused with a NS1 (murine myloma) cell line via typical PEG protocols, then plated into 16 plates.

### Screening for Reactive Clones:

Culture supernatants from all plates were screened by ELISA via peptides conjugated to EMCS-activated BSA. Ninety-four positive wells are selected from this initial test for expansion and a subsequent re-confirmation assay. Twenty positive wells from this test were selected to have one vial of cells frozen down, with supernatant saved for subsequent additional testing.

### Sub-Cloning:

Two positive parental cell lines were chosen to be further expanded and screened again by ELISA. The two best daughter clones from each were further expanded and isotyped (all clones were IgG1).

### Production:

Supernatant from one daughter clone (4F9.H5) was scaled up to 250 mL by roller-bottle production. The resultant material was purified by Protein G (Cytiva Life Sciences #P-00067) chromatography.

### Bcl10 and CARD9 Expression and Protein Purification:

Table 3 depicts the protein sequence of CARD9²⁻¹⁵² and MBP-Bcl10 constructs. The expressions and protein purifications were done according to the methods shown in (Holliday et al., 2019) with slight modifications. Modifications include lysing 72 hour CARD9²⁻¹⁵² pellets using an Avestin Microfluidizer under denaturing conditions. After the final Superdex S200 gel filtration step, CARD9²⁻¹⁵² protein was concentrated to two millimolars. 1 hour MBP-Bcl10 pellet was lysed in the nickel buffer using an Avestin Microfluidizer and post-Ni-NTA affinity column elutions were frozen down and stored at −80 C in one mL aliquots for subsequent fluorescence polarization assays.

TABLE 3
Protein Sequences of CARD9²⁻¹⁵² and
MBP-Bcl10 constructs

| | | | SEQ | |
|---|---|---|---|---|
| | | | ID | Protein |
| Vector | Plasmid | Insert | NO: | Sequence |
| pETite | pMD601 | CARD | 55 | SDYENDDECWSVL |
| N-His | | 9²⁻ | | EGFRVTLTSVIDP |
| Kan + | | 152/ | | SRITPYLRQCKVL |
| LacI | | WT | | NPDDEEQVLSDPN |
| (Lucigen) | | | | LVIRKRKVGVLLD |
| | | | | ILQRTGHKGYVAF |
| | | | | LESLELYYPQLYK |
| | | | | KVTGKEPARVFSM |
| | | | | IIDASGESGLTQL |
| | | | | LMTEVMKLQKKVQ |
| | | | | DLTALLSSKDDFI |
| | | | | KELRVKDS |
| | pMD602 | CARD | 56 | SDYENDDECWSVL |
| | | 9²⁻ | | EGFRVTLTSVIDP |
| | | 152/ | | SRITPYLRQCKVL |
| | | I107E | | NPDDEEQVLSDPN |
| | | | | LVIRKRKVGVLLD |
| | | | | ILQRTGHKGYVAF |
| | | | | LESLELYYPQLYK |
| | | | | KVTGKEPARVFSM |
| | | | | IEDASGESGLTQL |
| | | | | LMTEVMKLQKKVQ |
| | | | | DLTALLSSKDDFI |
| | | | | KELRVKDS |
| | pMD604 | CARD | 57 | SDYENDDECWSVL |
| | | 9²⁻ | | EGFRVTLTSVIDP |
| | | 152/ | | SRITPYLRQCKVL |
| | | S104D | | NPDDEEQVLSDPN |
| | | | | LVIRKRKVGVLLD |
| | | | | ILQRTGHKGYVAF |
| | | | | LESLELYYPQLYK |
| | | | | KVTGKEPARVFDM |
| | | | | IIDASGESGLTQL |
| | | | | LMTEVMKLQKKVQ |
| | | | | DLTALLSSKDDFI |
| | | | | KELRVKDS |
| | pMD605 | CARD | 58 | SDYENDDECWSVL |
| | | 9²⁻ | | EGFRVTLTSVIDP |
| | | 152/ | | SRITPYLRQCKVL |
| | | R101C | | NPDDEEQVLSDPN |
| | | | | LVIRKRKVGVLLD |
| | | | | ILQRTGHKGYVAF |
| | | | | LESLELYYPQLYK |
| | | | | KVTGKEPACVFSM |
| | | | | IIDASGESGLTQL |
| | | | | LMTEVMKLQKKVQ |
| | | | | DLTALLSSKDDFI |
| | | | | KELRVKDS |
| | pMD606 | CARD | 59 | SDYENDDECWSVL |
| | | 9²⁻ | | EGFRVTLTSVIDP |
| | | 152/ | | SRITPYLRQCKVL |
| | | R101C/ | | NPDDEEQVLSDPN |
| | | S104D | | LVIRKRKVGVLLD |
| | | | | ILQRTGHKGYVAF |
| | | | | LESLELYYPQLYK |
| | | | | KVTGKEPACVFDM |
| | | | | IIDASGESGLTQL |
| | | | | LMTEVMKLQKKVQ |
| | | | | DLTALLSSKDDFI |
| | | | | KELRVKDS |
| | pMD613 | Bcl10^{FL}/ | 60 | MEPTAPSLTEEDL |
| | | C29A/ | | TEVKKDALENLRV |
| | | C57A | | YLAEKIIAERHFD |
| | | | | HLRAKKILSREDT |
| | | | | EEISARTSSRKRA |
| | | | | GKLLDYLQENPKG |
| | | | | LDTLVESIRREKT |
| | | | | QNFLIQKITDEVL |
| | | | | KLRNIKLEHLKGL |
| | | | | KCSSCEPFPDGAT |
| | | | | NNLSRSNSDESNF |
| | | | | SEKLRASTVMYHP |
| | | | | EGESSTTPFFSTN |
| | | | | SSLNLPVLEVGRT |
| | | | | ENTIFSSTTLPRP |
| | | | | GDPGAPPLPPDLQ |
| | | | | LEEEGTCANSSEM |
| | | | | FLPLRSRTVS |

### BCL10 Fluorescence Polarization Assay

The fluorescence polarization assay preparations and assays were done according to the methods shown in (Holliday et al., 2019) with slight modifications. Two micromolar Alexa Fluor 488 C5 maleimide was added to one thawed aliquot of Bcl10 and purified over a Superdex 200 Increase gel filtration column. The monomeric Bcl10 peak was collected, stored at 4° C. in the dark and used in the fluorescent polarization assay within 1 hour of the purification. One to two millimolar of CARD9²⁻¹⁵² were incubated with 4 millimolar EDTA and shook at 500 rpm at 37° C. for 60 minutes to allow for filament formation. The assay was performed in 20 mM Tris, 150 mM NaCl, 0.5 mM TCEP, pH 7.5 in a final volume of 20 uL in a black 384-well plate. Two micromolar of MBP-Bcl10 and incubated CARD9 were added to the plate initially and 0.05 mg/mL TEV protease was added right before the fluorescence polarization readout. Fluorescence polarization was measured by exciting at 495 nm and monitoring at 520 nm on a PHERAstar FSX plate reader at 25° C.

scRNAseq

Single cell suspensions prepared as described above were processed using the Chromium Single Cell 3′ Gene Expression kit (v3.1, 10× Genomics) per manufacturer's instructions. Libraries were sequenced on the Illumina NovaSeq SP per manufacturer's instructions.

### Pre-Processing of Single-Cell RNA Sequencing Data

Pre-processing of raw fastq files (6 WT, 6 Card9 R101C) was conducted using Cell Ranger v.6.0.0 (1Ox genomics). Raw reads were aligned to the mm10 reference genome, and the Cell Ranger count function was used to generate UMI count matrices for each sample. Count matrices for each sample were aggregated prior to downstream processing. Cells with greater than 20% mitochondrial gene (“mt-”) expression were removed from the count matrix, followed by removal of all mitochondrial genes from the matrix. Ribosomal genes and the non-coding RNAs Neat1 and Malat1 were also removed. The distribution of log 10(mean gene UMI count) across all genes was examined, and log 10(library size) across all genes, and the matrix at −4.0 and 2.6 was filtered for each of these values, respectively, to remove genes with low read count and cells with abnormally low library size. 75647 cells and 20715 genes remained after these filters. The mean library size was 3948.41, and the mean number of genes captured per cell was 1455.63.

### Analysis of Single-Cell RNA Sequencing Data

The final count matrix of 75647 cells and 20715 genes was input into Seurat v4.0.1, (Hao et al., 2021), for downstream analysis. The top 10 principal components of the gene expression data were used as input to the FindNeighbors( ) function, after which we used RunUMAP( ) to generate a final dimensionality reduction of the data. This analysis yielded 17 clusters. Cluster enrichments in WT or R101C mice were calculated as proportion of all cells in each replicate, and statistics were evaluated using Dirichlet multinomial regression, as described in Smillie et al., 2019. Clusters were considered significantly differentially enriched in a condition at FDR P <0.1. We used the FindMarkers( ) function in Seurat to find DEGs. Genes were considered significantly differentially expressed at FDR P<0.05. DEG analysis was performed within the three broad lineages described in FIGS. 7A-7L (immune, fibroblast/endothelial cells, keratinocytes) and between WT and R101C within each cluster separately at D2 or D9, and/or between D2 and D9 for WT and R101C cells. Pathway analyses for WT vs R101C DEGs were conducted using the C5 pathway sets from MsigDB; pathways and DEGs were evaluated using gene set enrichment analysis (GSEA) as implemented in the fgsea( ) function in R. To align keratinocyte clusters identified in the present study with previously defined clusters from Joost et al., 2016, DEGs were obtained for all clusters from Joost et al., 2016 and enrichment of the cluster DEGs was evaluated in those clusters using fgsea( ). High normalized enrichment score (NES) indicates positively enriched; low NES indicates negatively enriched. The MitoCarta oxidative phosphorylation signature was applied to scRNA-seq data using the AddModuleScore( ) function in Seurat. For analyses In FIGS. 16H and 16I, the CARD9 DEGs overlapping with the wound healing (www.informatics.jax.org/go/term/GO:0042060) and inflammatory response (www.informatics.jax.org/go/term/GO:0006954) gene sets obtained from gene ontology were examined.

### Statistical Analysis

Mouse experiments included 4-10 mice per group and were repeated at least 2 times with data being reproducible between the repeats. Mice were randomly allocated into age and sex matched experimental groups. When possible, littermates were used. No outliers were excluded. Differences between experimental groups were assessed using paired t-test unless otherwise indicated. All statistical analysis was performed using GraphPad Prism v.9 unless otherwise specified. P value below 0.05 was considered statistically significant.

### OTHER EMBODIMENTS

From the foregoing description, it will be apparent that variations and modifications may be made to some aspects and embodiments herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Huang, L., Guo, Z., Wang, F. & Fu, L. KRAS mutation: from undruggable to druggable in cancer. Signal Transduct Target Ther 6, 386 (2021).
Bongomin, F., Gago, S., Oladele, R. O. & Denning, D. W. Global and multi-national prevalence of fungal diseases-estimate precision. J Fungi 3, 57 (2017).
Desoubeaux, G., Coste, A. T., Imbert, C. & Hennequin, C. Overview about Candida auris: What's up 12 years after its first description? Journal of Medical Mycology 32, 101248 (2022).
Firacative, C. Invasive fungal disease in humans: are we aware of the real impact? Mem Inst Oswaldo Cruz 115, e200430 (2020).
Rodrigues, M. L. & Nosanchuk, J. D. Fungal diseases as neglected pathogens: A wake-up call to public health officials. PLoS Negl Trop Dis 14, e0007964 (2020).
Fodil, N., Langlais, D. & Gros, P. Primary Immunodeficiencies and Inflammatory Disease: A Growing Genetic Intersection. Trends Immunol 37, 126-140 (2016).
Pathakumari, B., Liang, G. & Liu, W. Immune defence to invasive fungal infections: A comprehensive review. Biomedicine & Pharmacotherapy 130, 110550 (2020).
Kumar, V. et al. Immunochip SNP array identifies novel genetic variants conferring susceptibility to candidaemia. Nat Commun 5, 4675 (2014).
Vaezi, A. et al. Frequency and Geographic Distribution of CARD9 Mutations in Patients With Severe Fungal Infections. Front Microbiol 9, 2434 (2018).
Beaudoin, M. et al. Deep Resequencing of GWAS Loci Identifies Rare Variants in CARD9, IL23R and RNF186 That Are Associated with Ulcerative Colitis. PLoS Genet 9, e1003723 (2013).
Rivas, M. A. et al. Deep resequencing of GWAS loci identifies independent rare variants associated with inflammatory bowel disease. Nat Genet 43, 1066-1073 (2011).
Cao, Z. et al. Ubiquitin ligase TRIM62 regulates CARD9-mediated anti-fungal immunity and intestinal inflammation. Immunity 43, 715-26 (2015).
Pointon, J. J. et al. Elucidating the chromosome 9 association with AS; CARD9 is a candidate gene. Genes Immun 11, 490-496 (2010).
Janse, M. et al. Three ulcerative colitis susceptibility loci are associated with primary sclerosing cholangitis and indicate a role for IL2, REL, and CARD9. Hepatology 53, 1977-1985 (2011).
Kiryluk, K. et al. Discovery of new risk loci for IgA nephropathy implicates genes involved in immunity against intestinal pathogens. Nat Genet 46, 1187-1196 (2014). 10.1038/ng.3118.
Gross, O. et al. Card9 controls a non-TLR signalling pathway for innate anti-fungal immunity. Nature 442, 651-656 (2006).
Hara, H. et al. Cell type-specific regulation of ITAM-mediated NF-κB activation by the adaptors, CARMA1 and CARD9. The Journal of Immunology 181, 918-930 (2008).
Hara, H. et al. The adaptor protein CARD9 is essential for the activation of myeloid cells through ITAM-associated and Toll-like receptors. Nat Immunol 8, 619-629 (2007).
Drummond, R. A., Franco, L. M. & Lionakis, M. S. Human CARD9: a critical molecule of fungal immune surveillance. Front Immunol 9, 1836 (2018).
Strasser, D. et al. Syk kinase-coupled C-type lectin receptors engage protein kinase C-6 to elicit Card9 adaptor-mediated innate immunity. Immunity 36, 32-42 (2012).
Holliday, M. J. et al. Structures of autoinhibited and polymerized forms of CARD9 reveal mechanisms of CARD9 and CARD11 activation. Nat Commun 10, 3070 (2019).
Grumach, A. S. et al. A homozygous CARD9 mutation in a Brazilian patient with deep dermatophytosis. J Clin Immunol 35, 486-490 (2015).
Lanternier, F. et al. Deep dermatophytosis and inherited CARD9 deficiency. N Engl J Med 369, 1704-1714 (2013).
Johnson, J. L. et al. An atlas of substrate specificities for the human serine/threonine kinome. Nature 613, 759-766 (2023).
Xu, W. et al. USP15 deubiquitinates CARD9 to downregulate C-Type lectin receptor-mediated signaling. Immunohorizons 4, 670-678 (2020).
Qiao, Q. et al. Structural architecture of the CARMA1/Bcl10/MALT1 signalosome: nucleation-induced filamentous assembly. Mol Cell 51, 766-779 (2013).
Holliday, M. J. et al. Picomolar zinc binding modulates formation of Bcl10-nucleating assemblies of the caspase recruitment domain (CARD) of CARD9. J Biol Chem 293, 16803-16817 (2018).
Joost, S. et al. Single-cell transcriptomics reveals that differentiation and spatial signatures shape epidermal and hair follicle heterogeneity. Cell Syst 3, 221-237.e9 (2016).
Mantovani, A. et al. The chemokine system in diverse forms of macrophage activation and polarization. Trends Immunol 25, 677-686 (2004).
Ji, C., Yang, Z., Zhong, X. & Xia, J. The role and mechanism of CARD9 gene polymorphism in diseases. Biomed J 44, 560-566 (2021).
Drummond, R. A. & Lionakis, M. S. Mechanistic insights into the role of C-type lectin receptor/CARD9 signaling in human antifungal immunity. Front Cell Infect Microbiol 6, 39 (2016).
Gavino, C. et al. Impaired RASGRF1/ERK-mediated GM-CSF response characterizes CARD9 deficiency in French-Canadians. Journal of Allergy and Clinical Immunology 137, 1178-1188.e7 (2016).
de Bruyne, M. et al. A CARD9 founder mutation disrupts NF-κB signaling by inhibiting BCL10 and MALT1 recruitment and signalosome formation. Front Immunol 9, 2366 (2018).
Xu, X. et al. CARD9S12N facilitates the production of IL-5 by alveolar macrophages for the induction of type 2 immune responses. Nat Immunol 19, 547-560 (2018).
Nowarski, R., Jackson, R. & Flavell, R. A. The stromal intervention: regulation of immunity and inflammation at the epithelial-mesenchymal barrier. Cell 168, 362-375 (2017).
West, N. R. et al. Oncostatin M drives intestinal inflammation and predicts response to tumor necrosis factor-neutralizing therapy in patients with inflammatory bowel disease. Nat Med 23, 579-589 (2017).
Friedrich, M. et al. IL-1-driven stromal-neutrophil interactions define a subset of patients with inflammatory bowel disease that does not respond to therapies. Nat Med 27, 1970-1981 (2021).
Lionakis, M. S. et al. Organ-specific innate immune responses in a mouse model of invasive candidiasis. J Innate Immun 3, 180-199 (2011).
Freund, E. C. et al. Efficient gene knockout in primary human and murine myeloid cells by non-viral delivery of CRISPR-Cas9. J Exp Med 217, e20191692 (2020).
Hao, Y. et al. Integrated analysis of multimodal single-cell data. Cell 184, 3573-3587.e29 (2021).
Smillie, C. S. et al. Intra- and inter-cellular rewiring of the human colon during ulcerative colitis. Cell 178, 714-730.e22 (2019).

What is claimed is:

1. An antibody that specifically binds to a phosphorylated S104 amino acid residue in a caspase activation and recruitment domain 9 (CARD9) polypeptide or peptide, or an antigen binding portion thereof, said antibody comprising a heavy chain variable domain having at least about 85% identity to CDR1, CDR2, and CDR3 and a light chain variable domain having at least about 85% identity to CDR1, CDR2, and CDR3 of Table 1.

2. The antibody of claim 1, wherein the antibody comprises a heavy chain variable domain having at least about 90%, 95%, or 100% identity to CDR1, CDR2, and CDR3 and a light chain variable domain having at least about 90%, 95%, or 100% identity to CDR1, CDR2, and CDR3 of Table 1.

3. The antibody of claim 1, further comprising an affinity tag or a detectable moiety.

4. An isolated nucleic acid molecule encoding the antibody or antigen binding fragment of claim 1.

5. A vector comprising a nucleic acid sequence encoding the antibody of claim 1.

6. The vector of claim 5, wherein the vector is an expression vector.

7. The vector of claim 6, wherein the expression vector is a viral or non-viral expression vector.

8. The vector of claim 6, wherein the vector further comprises a nucleic acid sequence encoding an affinity tag or a detectable amino acid sequence operably linked to the antibody.

9. A host cell comprising the vector of claim 5.

10. A method of characterizing the activation state of a CARD9 polypeptide, the method comprising;
contacting a biological sample with an antibody or an antigen-binding portion thereof, of claim 1; and
detecting or failing to detect binding of the antibody or an antigen-binding portion thereof to a phosphorylated or unphosphorylated S104 of CARD9 in the sample, thereby characterizing the activation state of a CARD9 polypeptide.

11. The method of claim 10, wherein the antibody specifically binds a phosphorylated S104 amino acid residue present in CARD9, wherein said binding detects activated CARD9.

12. The method of claim 10, wherein the antibody fails to bind a phosphorylated S104 amino acid residue present in CARD9, wherein the failure to bind detects unactivated CARD9.

13. The method of claim 12, wherein detecting an activated CARD9 polypeptide indicates that the subject is capable of mounting an adequate immune response to a fungal infection, and/or wherein failing to detect an activated CARD9 polypeptide indicates that the subject has a propensity to develop a severe fungal infection.

14. A method of treating a selected subject having a fungal infection, the method comprising:
administering to the selected subject an aggressive anti-fungal therapy, wherein the subject is selected for treatment by detecting in a biological sample of the subject a reduction in the level of CARD9 polypeptide in which amino acid residue S104 is phosphorylated, relative to a reference.

15. The method of claim 14, wherein the detecting comprises contacting a biological sample of the subject with the antibody of claim 1, and detecting or failing to detect binding of the antibody to a CARD9 polypeptide present in the biological sample.

16. A method of selecting a subject having a propensity to develop a severe fungal infection, the method comprising:
(a) contacting a biological sample obtained from the subject with an antibody of claim 1, or an antigen-binding portion thereof, that specifically binds to a phosphorylated S104 amino acid residue in a CARD9 polypeptide;
(b) detecting a reduction in binding levels of the antibody, or an antigen-binding portion thereof, to a phosphorylated S104 amino acid residue in the CARD9 polypeptide, relative to a reference; and
(c) selecting the subject as having a propensity to develop an impaired immune response associated with the fungal infection based on the detecting step (b).

17. A method of selecting a subject having, or having a propensity to develop, Crohn's disease, inflammatory bowel disease, ankylosing spondylitis, primary sclerosing cholangitis, or IgA nephropathy, the method comprising:
(a) contacting a biological sample obtained from the subject with an antibody of claim 1, or an antigen-binding portion thereof, that specifically binds to a phosphorylated S104 amino acid residue in a CARD9 polypeptide;
(b) detecting an increase in binding levels of the antibody, or an antigen-binding portion thereof, to a phosphorylated S104 amino acid residue in the CARD9 polypeptide, relative to a reference; and
(c) selecting the subject as having, or having a propensity to develop, Crohn's disease, inflammatory bowel disease, ankylosing spondylitis, primary sclerosing cholangitis, or IgA nephropathy based on the detecting step (b).

18. The method of claim 17, further comprising administering to the selected subject an immunosuppressive or immunomodulatory agent.

19. A method of screening for an agent that activates a CARD9 polypeptide, the method comprising:
contacting a sample with an agent and with the antibody or an antigen-binding portion thereof, of claim 1 under conditions and for a time sufficient for binding of the antibody or an antigen binding portion thereof to bind to the CARD9 protein if present in the sample;
identifying the agent as an activator of the CARD9 polypeptide by detecting the binding of the antibody or an antigen binding portion thereof, to the CARD9 polypeptide in the sample compared with a control sample in which the CARD9 protein is absent or in which the CARD9 protein is unphosphorylated.

20. A method of screening for an agent that inhibits the activation of a CARD9 polypeptide, the method comprising:
contacting a sample with an agent and with the antibody or an antigen-binding portion thereof, of claim 1 under conditions and for a time sufficient for binding of the antibody or an antigen binding portion thereof to bind to the CARD9 protein if present in the sample;
identifying the agent as an inhibitor of the activation of the CARD9 polypeptide by detecting a reduction in the binding of the antibody or an antigen binding portion thereof, to the CARD9 polypeptide in the sample compared with a control sample in which the CARD9 protein is activated or in which the CARD9 protein is phosphorylated.
